(19) United States
(12) Patent Application Publication
(10) Pub. No.: US 2007/0135387 A1
(43) Pub. Date: Jun. 14, 2007

(54) Inhibitors of protein kinases

(76) Inventors: Michael R. Michaelides, Libertyville, IL (US); William J. McClellan, Waukegan, IL (US); Robin R. Frey, Libertyville, IL (US); Michael L. Curtin, Pleasant Prairie, WI (US); Douglas H. Steinman, Morton Grove, IL (US); Yujia Dai, Gurnee, IL (US); James H. Holms, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/636,189

(22) Filed: Dec. 8, 2006

Related U.S. Application Data
(60) Provisional application No. 60/749,074, filed on Dec. 8, 2005.

(51) Int. Cl. A61K 31/675; A61K 31/519; C07D 487/02; C07F 9/6512

(52) U.S. Cl. 514/80; 514/260.1; 514/265.1; 544/278; 544/280; 544/244

(57) ABSTRACT

Compounds that inhibit protein kinases, compositions containing the compounds and methods of treating diseases using the compounds are disclosed.

This application claims priority to U.S. Provisional Parent Application Ser. No. 60/749,074, filed Dec. 8, 2005.

### FIELD OF THE INVENTION

This invention pertains to compounds that inhibit protein kinases such as Aurora kinases, compositions containing the compounds and methods of treating diseases using the compounds.

### BACKGROUND OF THE INVENTION

Mitosis is a process by which a complete copy of a duplicated genome is segregated by the microtuble spindle apparatus into two daughter cells. Aurora kinases, key mitotic regulators required for genome stability, have been found to be overexpressed in human tumors. Given the central role of mitosis in the progression of maligncies, inhibitors of mitosis are expected to be useful for treating a broad range of tumors.

There is therefore an existing need in the therapeutic arts for inhibitors of Aurora kinases.

### SUMMARY OF THE INVENTION

One embodiment of this invention, therefore, pertains to compounds that inhibit Aurora kinases, the compounds having formula (I)

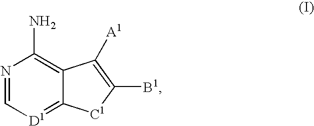

A¹ is C(O)NHR¹, C(O)N(R¹)₂, NHC(O)R¹, NR¹C(O)R¹, NHC(O)NHR¹, NHC(O)N(R¹)₂, NR¹C(O)NHR¹, NR¹C(O)N(R¹)₂, SO₂NHR¹, SO₂N(R¹)₂, NHSO₂R¹, NR¹SO₂R¹, OC(O)OR¹, NHC(O)OR¹, NR¹C(O)OR¹ or R⁵;

R¹ is R², R³, R⁴ or R⁵;

R² is phenyl which is unfused or fused with benzene, heteroarene or R^{2A}; R^{2A} is cycloalkane, cycloalkene, heterocycloalkane or heterocycloalkene;

R³ is heteroaryl which is unfused or fused with benzene, heteroarene or R^{3A}; R^{3A} is cycloalkane, cycloalkene, heterocycloalkane or heterocycloalkene;

R⁴ is cycloalkyl, cycloalkenyl, heterocycloalkyl or heterocycloalkenyl, each of which is unfused or fused with benzene, heteroarene or R^{4A}; R^{4A} is cycloalkane, cycloalkene, heterocycloalkane or heterocycloalkene;

R⁵ is alkyl, alkenyl or alkynyl, each of which is substituted with one or two of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHSO₂R⁶, NR⁶SO₂R⁶, NHC(O)OR⁶, NR⁶C(O)OR⁶, SO₂NH₂, SO₂NHR⁶, SO₂N(R⁶)₂, NHC(O)NH₂, NHC(O)NHR⁶, NHC(O)N(R⁶)₂, NR⁶C(O)N(R⁶)₂, OH, (O), C(O)OH, CN, NH₂, CF₃, OCF₃, CF₂CF₃, F, Cl, Br or I;

R⁶ is R⁷, R⁸ or R⁹;

R⁷ is phenyl which is unfused or fused with benzene, heteroarene or R^{7A}; R^{7A} is cycloalkane, cycloalkene, heterocycloalkane or heterocycloalkene;

R⁸ is heteroaryl which is unfused or fused with benzene, heteroarene or R^{8A}; R^{8A} is cycloalkane, cycloalkene, heterocycloalkane or heterocycloalkene;

R⁹ is cycloalkyl, cycloalkenyl, heterocycloalkyl or heterocycloalkenyl, each of which is unfused or fused with benzene, heteroarene or R^{9A}; R^{9A} is cycloalkane, cycloalkene, heterocycloalkane or heterocycloalkene;

wherein the moieties represented by R¹, R², R³, R⁴, R⁵ and R⁶ are independently substituted with one or two of independently selected R¹⁰, OR¹⁰, SR¹⁰, S(O)R¹⁰, SO₂R¹⁰, NH₂, NHR¹⁰, N(R¹⁰)₂, C(O)R¹⁰, C(O)OR¹⁰, C(O)NHR¹⁰, C(O)N(R¹⁰)₂, NHC(O)R¹⁰, NR¹⁰C(O)R^{10,}NHC(O)NHR¹⁰, NHC(O)N(R¹⁰)₂, NR¹⁰C(O)NHR¹⁰, NR¹⁰C(O)N(R¹⁰)₂, SO₂NHR¹⁰, SO₂N(R¹⁰)₂, NHSO₂R¹⁰, NR¹SO₂R¹⁰, OC(O)OR¹⁰, NHC(O)OR¹⁰ or NR¹C(O)OR¹⁰;

R¹⁰ is R¹¹, R¹², R¹³ or R¹⁴;

R¹¹ is phenyl which is unfused or fused with benzene, heteroarene or R^{11A}; R^{11A} is cycloalkane, cycloalkene, heterocycloalkane or heterocycloalkene;

R¹² is heteroaryl which is unfused or fused with benzene, heteroarene or R^{12A}; R^{12A} is cycloalkane, cycloalkene, heterocycloalkane or heterocycloalkene;

R¹³ is cycloalkyl, cycloalkenyl, heterocycloalkyl or heterocycloalkenyl, each of which is unfused or fused with benzene, heteroarene or R^{13A}; R^{13A} is cycloalkane, cycloalkene, heterocycloalkane or heterocycloalkene;

R¹⁴ is alkyl, alkenyl or alkynyl, each of which is unsubstituted or substituted with one or two of independently selected R¹⁵ or NHC(O)NHR¹⁵;

R¹⁵ is R¹⁶, R¹⁷ R¹⁸;

R¹⁶ is phenyl which is unfused or fused with benzene, heteroarene or R^{16A}; R^{16A} is cycloalkane, cycloalkene, heterocycloalkane or heterocycloalkene;

R¹⁷ is heteroaryl which is unfused or fused with benzene, heteroarene or R^{17A}; R^{17A} is cycloalkane, cycloalkene, heterocycloalkane or heterocycloalkene;

R¹⁸ is cycloalkyl, cycloalkenyl, heterocycloalkyl or heterocycloalkenyl each of which is unfused or fused with benzene, heteroarene or R^{18A}; R^{18A} is cycloalkane, cycloalkene, heterocycloalkane or heterocycloalkene;

B¹ is H, R¹⁹, C(O)NHR¹⁹, C(O)N(R¹⁹)₂, NHC(O)R¹⁹, NR¹C(O)R¹⁹, NHC(O)NHR¹⁹, NHC(O)N(R¹⁹)₂, NR¹⁹C(O)NHR¹⁹, NR¹C(O)N(R¹⁹)₂, SO₂NHR¹⁹, SO₂N(R¹⁹)₂, NHSO₂R¹⁹, NR¹⁹SO₂R¹⁹, OC(O)OR¹⁹, NHC(O)OR¹⁹, or NR¹⁹C(O)OR¹⁹;

R¹⁹ is R²⁰, R²¹, R²² or R²³;

R²⁰ is phenyl which is unfused or fused with benzene, heteroarene or R^{20A}; R^{20A} is cycloalkane, cycloalkene, heterocycloalkane or heterocycloalkene;

R²¹ is heteroaryl which is unfused or fused with benzene, heteroarene or R^{21A}; R^{21A} is cycloalkane, cycloalkene, heterocycloalkane or heterocycloalkene;

R²² is cycloalkyl, cycloalkenyl, heterocycloalkyl or heterocycloalkenyl, each of which is unfused or fused with benzene, heteroarene or R^{22A}; R^{22A} is cycloalkane, cycloalkene, heterocycloalkane or heterocycloalkene;

R²³ is alkyl, alkenyl or alkynyl, each of which is unsubstituted or substituted with one or two of independently selected R²⁴, OR²⁴, SR²⁴, S(O)R²⁴, SO₂R²⁴, NH₂, NHR²⁴, N(R²⁴)₂, C(O)R²⁴, C(O)NH₂, C(O)NHR²⁴, C(O)N(R²⁴)₂, NHC(O)R²⁴, NR²⁴C(O)R²⁴, NHSO²⁴R²⁴, NR²⁴ SO₂R²⁴, NHC(O)OR²⁴, NR²⁴C(O)OR²⁴, SO₂NH₂, SO₂NHR²⁴, SO₂N(R²⁴)₂, NHC(O)NH₂, NHC(O)NHR²⁴, NHC(O)N(R²⁴)₂, NR²⁴C(O)N(R²⁴)₂, OH, (O), C(O)OH, CN, NH₂, CF₃, OCF₃, CF₂CF₃, F, Cl, Br or I;

R²⁴ is R²⁵, R²⁶, R²⁷, alkyl, alkenyl or alkynyl;

R²⁵ is phenyl which is unfused or fused with benzene, heteroarene or R^{25A}; R^{25A} is cycloalkane, cycloalkene, heterocycloalkane or heterocycloalkene;

R²⁶ is heteroaryl which is unfused or fused with benzene, heteroarene or R^{26A}; R^{26A} is cycloalkane, cycloalkene, heterocycloalkane or heterocycloalkene;

R²⁷ is cycloalkyl, cycloalkenyl, heterocycloalkyl or heterocycloalkenyl, each of which is unfused or fused with benzene, heteroarene or R^{27A}; R^{27A} is cycloalkane, cycloalkene, heterocycloalkane or heterocycloalkene;

C¹ is O, S, S(O), SO₂, NH, or N(C²);

C² is R²⁸, R²⁹, R³⁰ or R³¹;

R²⁸ is phenyl which is unfused or fused with benzene, heteroarene or R^{28A}; R^{28A} is cycloalkane, cycloalkene, heterocycloalkane or heterocycloalkene;

R²⁹ is heteroaryl which is unfused or fused with benzene, heteroarene or R^{29A}; R^{29A} is cycloalkane, cycloalkene, heterocycloalkane or heterocycloalkene;

R³⁰ is cycloalkyl, cycloalkenyl, heterocycloalkyl or heterocycloalkenyl, each of which is unfused or fused with benzene, heteroarene or R^{30A}; R^{30A} is cycloalkane, cycloalkene, heterocycloalkane or heterocycloalkene;

R³¹ is alkyl, alkenyl or alkynyl, each of which is substituted with one or two of independently selected R³², OR³², SR³², S(O)R³², SO₂R³², NH₂, NHR³², N(R³²)₂, C(O)R³², C(O)NH₂, C(O)NHR³², C(O)N(R³²)₂, NHC(O)R³², NR³²C(O)R³², NHSO₂R³², NR³²SO₂R³², NHC(O)OR³², NR³²C(O)OR³², SO₂NH₂, SO₂NHR³², SO₂N(R³²)₂, NHC(O)NH₂, NHC(O)NHR³², NHC(O)N(R³²)₂, NR³²C(O)N(R³²)₂, OH, (O), C(O)OH, CN, NH₂, CF₃, OCF₃, CF₂CF₃, F, Cl, Br or I;

R³² is R³³, R³⁴ or R³⁵;

R³³ is phenyl which is unfused or fused with benzene, heteroarene or R^{33A}; R^{33A} is cycloalkane, cycloalkene, heterocycloalkane or heterocycloalkene;

R³⁴ is heteroaryl which is unfused or fused with benzene, heteroarene or R^{34A}; R^{34A} is cycloalkane, cycloalkene, heterocycloalkane or heterocycloalkene;

R³⁵ is cycloalkyl, cycloalkenyl, heterocycloalkyl or heterocycloalkenyl, each of which is unfused or fused with benzene, heteroarene or R^{35A}; R^{35A} is cycloalkane, cycloalkene, heterocycloalkane or heterocycloalkene;

D¹ is N, CH or C(D²);

D² is R³⁶, R³⁷, R³⁸ or R³⁹;

R³⁶ is phenyl which is unfused or fused with benzene, heteroarene or R^{36A}; R^{36A} is cycloalkane, cycloalkene, heterocycloalkane or heterocycloalkene;

R³⁷ is heteroaryl which is unfused or fused with benzene, heteroarene or R^{37A}; R^{37A} is cycloalkane, cycloalkene, heterocycloalkane or heterocycloalkene;

R³⁸ is cycloalkyl, cycloalkenyl, heterocycloalkyl or heterocycloalkenyl, each of which is unfused or fused with benzene, heteroarene or R^{38A}; R^{38A} is cycloalkane, cycloalkene, heterocycloalkane or heterocycloalkene;

R³⁹ is alkyl, alkenyl or alkynyl, each of which is substituted with one or two of independently selected R⁴⁰, OR⁴⁰, SR⁴⁰, S(O)R⁴⁰, SO₂R⁴⁰, NH₂, NHR⁴⁰, N(R⁴⁰)₂, C(O)R⁴⁰, C(O)NH₂, C(O)NHR⁴⁰, C(O)N(R⁴⁰)₂, NHC(O)R⁴⁰, NR⁴⁰C(O)R⁴⁰, NHSO₂R⁴⁰, NR⁴⁰SO₂R⁴⁰, NHC(O)OR⁴⁰, NR⁴⁰C(O)OR⁴⁰, SO₂NH₂, SO₂NHR⁴⁰, SO₂N(R⁴⁰)₂, NHC(O)NH₂, NHC(O)NHR⁴⁰, NHC(O)N(R⁴⁰)₂, NR⁴⁰C(O)N(R⁴⁰)₂, OH, (O), C(O)OH, CN, NH₂, CF₃, OCF₃, CF₂CF₃, F, Cl, Br or I;

R⁴⁰ is R⁴¹, R⁴² or R⁴³;

R⁴¹ is phenyl which is unfused or fused with benzene, heteroarene or R^{41A}; R^{41A} is cycloalkane, cycloalkene, heterocycloalkane or heterocycloalkene;

R⁴² is heteroaryl which is unfused or fused with benzene, heteroarene or R^{42A}; R^{42A} is cycloalkane, cycloalkene, heterocycloalkane or heterocycloalkene;

R⁴³ is cycloalkyl, cycloalkenyl, heterocycloalkyl or heterocycloalkenyl, each of which is unfused or fused with benzene, heteroarene or R^{43A}; R^{43A} is cycloalkane, cycloalkene, heterocycloalkane or heterocycloalkene;

wherein each foregoing cyclic moiety is independently unsubstituted or substituted or further unsubstituted or further substituted with one or two or three or four of independently selected R⁴⁴, OR⁴⁴, SR⁴⁴, S(O)R⁴⁴, SO₂R⁴⁴, NH₂, NHR⁴⁴, N(R⁴⁴)₂, C(O)OR⁴⁴, C(O)NH₂, C(O)NHR⁴⁴, C(O)N(R⁴⁴)₂, NHC(O)R⁴⁴, NR⁴⁴C(O)R⁴⁴, NHSO₂R⁴⁴, NR⁴⁴SO₂R⁴⁴, NHC(O)OR⁴⁴, NR⁴⁴C(O)OR⁴⁴, SO₂NH₂, SO₂NHR⁴⁴, SO₂N(R⁴⁴)₂, NHC(O)NH₂, NHC(O)NHR⁴⁴, NHC(O)N(R⁴⁴)₂, NR⁴⁴C(O)N(R⁴⁴)₂, C(N)NH₂, C(N)NHR⁴⁴, C(N)N(R⁴⁴)₂, NHC(N)NH₂, NHC(N)NHR⁴⁴, NHC(N)N(R⁴⁴)₂, OH, (O), C(O)H, C(O)OH, NO₂, CN, CF₃, OCF₃, CF₂CF₃, F, Cl, Br or I;

R⁴⁴ is R⁴⁵, R⁴⁶, R⁴⁷ or R⁴⁸;

R⁴⁵ is phenyl which is unfused or fused with benzene, heteroarene or R^{45A}; R^{45A} is cycloalkane, cycloalkene, heterocycloalkane or heterocycloalkene;

R⁴⁶ is heteroaryl which is unfused or fused with benzene, heteroarene or R^{46A}; R^{46A} is cycloalkane, cycloalkene, heterocycloalkane or heterocycloalkene;

R⁴⁷ is cycloalkyl, cycloalkenyl, heterocycloalkyl or heterocycloalkenyl, each of which is unfused or fused with benzene, heteroarene or R^{47A}; R^{47A} is cycloalkane, cycloalkene, heterocycloalkane or heterocycloalkene;

R⁴⁸ is alkyl, alkenyl or alkynyl, each of which is substituted with one or two of independently selected R⁴⁹, OR⁴⁹, SR⁴⁹, S(O)R⁴⁹, SO₂R⁴⁹, NH₂, NHR⁴⁹, N(R⁴⁹)₂, C(O)R⁴⁹, C(O)NH₂, C(O)NHR⁴⁹, C(O)N(R⁴⁹)₂, NHC(O)R⁴⁹, NR⁴⁹C(O)R⁴⁹, NHSO₂R⁴⁹, NR⁴⁹SO₂R⁴⁹, NHC(O)OR⁴⁹, NR⁴⁹C(O)OR⁴⁹, SO₂NH₂, SO₂NHR⁴⁹, SO₂N(R⁴⁹)₂, NHC(O)NH₂, NHC(O)NHR⁴⁹, NHC(O)N(R⁴⁹)₂, NR⁴⁹C(O)N(R⁴⁹)₂, OP(O)(OH)₂, OP(O)(OH)(OR⁴⁴), OP(O)(OR⁴⁴)₂, OH, (O), C(O)OH, CN, CF₃, OCF₃, CF₂CF₃, F, Cl, Br or I;

R⁴⁹ is R⁵⁰, R⁵¹, R⁵², alkyl, alkenyl or alkynyl;

R⁵⁰ is phenyl which is unfused or fused with benzene, heteroarene or R^{50A}; R^{50A} is cycloalkane, cycloalkene, heterocycloalkane or heterocycloalkene;

R⁵¹ is heteroaryl which is unfused or fused with benzene, heteroarene or R^{51A}; R^{51A} is cycloalkane, cycloalkene, heterocycloalkane or heterocycloalkene; and

R⁵² is cycloalkyl, cycloalkenyl, heterocycloalkyl or heterocycloalkenyl, each of which is unfused or fused with benzene, heteroarene or R^{52A}; R^{52A} is cycloalkane, cycloalkene, heterocycloalkane or heterocycloalkene;

wherein the moieties represented by R⁴⁵, R⁴⁶, R⁴⁷ and R⁴⁹ are independently unsubstituted or substituted with one or two or three of four of independently selected alkyl, alkenyl, alkynyl, OH, (O), C(O)OH, CN, CF₃, OCF₃, CF₂CF₃, F. Cl, Br or I.

Another embodiment comprises compounds having formula (I), and therapeutically acceptable salts, prodrugs and salts of prodrugs thereof, wherein A¹ is C(O)NHR¹, C(O)N(R¹)₂, NHC(O)R¹, NR¹C(O)R¹, NHC(O)NHR¹, NHC(O)N(R¹)₂, NR¹C(O)NHR¹, NR¹C(O)N(R¹)₂, SO₂NHR¹, SO₂N(R¹)₂, NHSO₂R¹, NR¹SO₂R¹, OC(O)OR¹, NHC(O)OR¹, NR¹C(O)OR¹ or R⁵;

R¹ is R², R³ or R⁴;

R² is phenyl which is unfused or fused with benzene or heteroarene;

R³ is heteroaryl which is unfused or fused with benzene or heteroarene;

R⁴ is cycloalkyl, cycloalkenyl, heterocycloalkyl or heterocycloalkenyl, each of which is unfused or fused with benzene or heteroarene;

R⁵ is alkyl, alkenyl or alkynyl, each of which is substituted with one or two of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHC(O)NHR⁶, OH, (O), C(O)OH, CN, NH₂, CF₃, OCF₃, CF₂CF₃, F, Cl, Br or I;

R⁶ is R⁷, R⁸ or R⁹;

R⁷ is phenyl which is unfused or fused with benzene or heteroarene;

R⁸ is heteroaryl which is unfused or fused with benzene or heteroarene:

R⁹ is cycloalkyl, cycloalkenyl, heterocycloalkyl or heterocycloalkenyl, each of which is unfused or fused with benzene or heteroarene;

wherein the moieties represented by R¹, R², R³, R⁴, R⁵ and R⁶ are independently substituted with one or two of independently selected R¹⁰, OR¹⁰, SR¹⁰, S(O)R¹⁰, SO₂R¹⁰, NH₂, NHR¹⁰, N(R¹⁰)₂, C(O)R¹⁰, C(O)OR¹⁰, C(O)NHR¹⁰, C(O)N(R¹⁰)₂, NHC(O)R¹⁰, NR¹⁰C(O)R¹⁰ or NHC(O)NHR¹⁰;

R¹⁰ is R¹¹, R¹², R¹³ or R¹⁴;

R¹¹ is phenyl which is unfused or fused with benzene or heteroarene;

R¹² is heteroaryl which is unfused or fused with benzene or heteroarene;

R¹³ is cycloalkyl, cycloalkenyl, heterocycloalkyl or heterocycloalkenyl, each of which is unfused or fused with benzene or heteroarene;

R¹⁴ is alkyl, alkenyl or alkynyl, each of which is unsubstituted or substituted with one or two of independently selected R¹⁵ or NHC(O)NHR¹⁵;

R¹⁵ is R¹⁶, R¹⁷, R¹⁸;

R¹⁶ is phenyl which is unfused or fused with benzene or heteroarene;

R¹⁷ is heteroaryl which is unfused or fused with benzene or heteroarene;

R¹⁸ is cycloalkyl, cycloalkenyl, heterocycloalkyl or heterocycloalkenyl, each of which is unfused or fused with benzene or heteroarene;

B¹ is H or R¹⁹;

R¹⁹ is R²⁰, R²¹, R²² or R²³;

R²⁰ is phenyl which is unfused or fused with benzene or heteroarene;

R²¹ is heteroaryl which is unfused or fused with benzene or heteroarene;

R²² is cycloalkyl, cycloalkenyl, heterocycloalkyl or heterocycloalkenyl, each of which is unfused or fused with benzene or heteroarene;

R²³ is alkyl, alkenyl or alkynyl, each of which is unsubstituted or substituted with one or two of independently selected R²⁴, OR²⁴, N(R²⁴)₂, C(O)N(R²⁴)₂, NHC(O)R²⁴, NR²⁴C(O)R²⁴;

R²⁴ is alkyl, alkenyl or alkynyl;

C¹ is O, S, S(O), SO₂, NH, or N(C²);

C² is R²⁸, R²⁹ or R³⁰;

R²⁸ is phenyl which is unfused or fused with benzene or heteroarene;

R²⁹ is heteroaryl which is unfused or fused with benzene or heteroarene;

R³⁰ is cycloalkyl, cycloalkenyl, heterocycloalkyl or heterocycloalkenyl, each of which is unfused or fused with benzene or heteroarene;

D¹ is N, CH or C(D²);

D² is R³⁶, R³⁷ or R³⁸;

R³⁶ is phenyl which is unfused or fused with benzene or heteroarene;

R³⁷ is heteroaryl which is unfused or fused with benzene or heteroarene;

R³⁸ is cycloalkyl, cycloalkenyl, heterocycloalkyl or heterocycloalkenyl, each of which is unfused or fused with benzene or heteroarene;

wherein each foregoing cyclic moiety is independently unsubstituted or substituted or further unsubstituted or further substituted with one or two or three or four of independently selected R⁴⁴, OR⁴⁴, SR⁴⁴, S(O)R⁴⁴, SO₂R⁴⁴, NH₂, NHR⁴⁴, N(R⁴⁴)₂, C(O)R⁴⁴, C(O)OR⁴⁴, C(O)NH₂, C(O)NHR⁴⁴, C(O)N(R⁴⁴)₂, NHC(O)R⁴⁴, OH, (O), C(O)H, C(O)OH, NO₂, CN, CF₃, OCF₃, CF₂CF₃, F, Cl, Br or I;

R⁴⁴ is R⁴⁵, R⁴⁶, R⁴⁷ or R⁴⁸;

R⁴⁵ is phenyl which is unfused or fused with benzene or heteroarene;

R⁴⁶ is heteroaryl which is unfused or fused with benzene or heteroarene;

R⁴⁷ is cycloalkyl, cycloalkenyl, heterocycloalkyl or heterocycloalkenyl, each of which is unfused or fused with benzene or heteroarene;

R⁴⁸ is alkyl substituted with OP(O)(OH)₂;

wherein the moieties represented by R⁴⁵, R⁴⁶ and R⁴⁷ are independently unsubstituted or substituted with one or two or three or four of independently selected alkyl, alkenyl, alkynyl, OH, (O), C(O)OH, CN, CF₃, OCF₃, CF₂CF₃, F, Cl, Br or I.

Still another embodiment comprises compounds having formula (I), and therapeutically acceptable salts, prodrugs and salts of prodrugs thereof, wherein A¹ is C(O)NHR¹ or R⁵;

R¹ is R², R³ or R⁴;

R² is phenyl which is unfused or fused with benzene or heteroarene;

R³ is heteroaryl which is unfused or fused with benzene or heteroarene;

R⁴ is cycloalkyl, cycloalkenyl, heterocycloalkyl or heterocycloalkenyl, each of which is unfused or fused with benzene or heteroarene;

R⁵ is alkyl, alkenyl or alkynyl, each of which is substituted with one or two of independently selected R⁶, NHC(O)NHR⁶;

R⁶ is R⁷, R⁸ or R⁹;

R⁷ is phenyl which is unfused or fused with benzene or heteroarene;

R⁸ is heteroaryl which is unfused or fused with benzene or heteroarene;

R⁹ is cycloalkyl, cycloalkenyl, heterocycloalkyl or heterocycloalkenyl, each of which is unfused or fused with benzene or heteroarene;

wherein the moieties represented by R¹, R², R³, R⁴, R⁵ and R⁶ are independently substituted with one or two of independently selected R¹⁰, OR¹⁰, SR¹⁰, S(O)R¹⁰, SO₂R¹⁰, NH₂, NHC(O)R¹⁰, NHC(O)NHR¹⁰;

R¹⁰ is R¹¹, R¹², R¹³ or R¹⁴;

R¹¹ is phenyl which is unfused or fused with benzene or heteroarene;

R¹² is heteroaryl which is unfused or fused with benzene or heteroarene;

R¹³ is cycloalkyl, cycloalkenyl, heterocycloalkyl or heterocycloalkenyl, each of which is unfused or fused with benzene or heteroarene;

R¹⁴ is alkyl, alkenyl or alkynyl, each of which is unsubstituted or substituted with one or two of independently selected R¹⁵ or NHC(O)NHR¹⁵;

R¹⁵ is R¹⁶, R¹⁷ R¹⁸;

R¹⁶ is phenyl which is unfused or fused with benzene or heteroarene;

R¹⁷ is heteroaryl which is unfused or fused with benzene or heteroarene;

R¹⁸ is cycloalkyl, cycloalkenyl, heterocycloalkyl or heterocycloalkenyl, each of which is unfused or fused with benzene or heteroarene;

B¹ is H or R¹⁹;

R¹⁹ is R²⁰, R²¹, R²² or R²³;

R²⁰ is phenyl which is unfused or fused with benzene or heteroarene;

R²¹ is heteroaryl which is unfused or fused with benzene or heteroarene;

R²² is cycloalkyl, cycloalkenyl, heterocycloalkyl or heterocycloalkenyl, each of which is unfused with benzene or heteroarene;

R²³ is alkyl, alkenyl or alkynyl, each of which is unsubstituted or substituted with one or two of independently selected R²⁴, OR²⁴ or N(R²⁴)₂;

R²⁴ is alkyl, alkenyl or alkynyl;

C¹ is O, S, S(O), SO₂, NH, or N(C²);

C² is R²⁸, R²⁹ or R³⁰;

R²⁸ is phenyl which is unfused or fused with benzene or heteroarene;

R²⁹ is heteroaryl which is unfused or fused with benzene or heteroarene;

R³⁰ is cycloalkyl, cycloalkenyl, heterocycloalkyl or heterocycloalkenyl, each of which is unfused or fused with benzene or heteroarene;

D¹ is N, CH or C(D²);

D² is R³⁶, R³⁷ or R³⁸;

R³⁶ is phenyl which is unfused or fused with benzene or heteroarene;

R³⁷ is heteroaryl which is unfused or fused with benzene or heteroarene;

R³⁸ is cycloalkyl, cycloalkenyl, heterocycloalkyl or heterocycloalkenyl, each of which is unfused or fused with benzene or heteroarene;

wherein each foregoing cyclic moiety is independently unsubstituted or substituted or further unsubstituted or further substituted with one or two or three or four of independently selected R⁴⁴, OR⁴⁴, CN, CF₃, OCF₃, CF₂CF₃, F, Cl, Br or I;

R⁴⁴ is R45, R⁴⁶, R⁴⁷ or R⁴⁸;

R⁴⁵ is phenyl which is unfused or fused with benzene or heteroarene;

R⁴⁶ is heteroaryl which is unfused or fused with benzene or heteroarene;

R⁴⁷ is cycloalkyl, cycloalkenyl, heterocycloalkyl or heterocycloalkenyl, each of which is unfused or fused with benzene or heteroarene;

R⁴⁸ is alkyl substituted with OP(O)(OH)₂;

wherein the moieties represented by R⁴⁵, R⁴⁶, and R⁴⁷ are independently unsubstituted or substituted with one or two or three of four of independently selected alkyl.

Still another embodiment comprises compounds having formula (I), and therapeutically acceptable salts, prodrugs and salts of prodrugs thereof, wherein A¹ is C(O)NHR¹ or R⁵;

R¹ is R², R³ or R⁴;

R² is phenyl;

R³ is heteroaryl;

R⁴ is cycloalkyl or heterocycloalkyl;

R⁵ is alkyl, alkenyl or alkynyl, each of which is substituted with R⁶, NHC(O)NHR⁶;

R⁶ is R⁷ or R⁹;

R⁷ is phenyl;

R⁸ is heteroaryl;

R⁹ is heterocycloalkyl;

wherein the moieties represented by R¹, R², R³, R⁴, R⁵ and R⁶ are independently substituted with one or two of independently selected R¹⁰, OR¹⁰, SR¹⁰, SO₂R¹⁰, NH₂, NHC(O)R¹⁰, NHC(O)NHR¹⁰;

R¹⁰ is R¹¹, R¹², R¹³ or R¹⁴;

R¹¹ is phenyl;

R¹² is heteroaryl;

R¹³ is cycloalkyl;

R¹⁴ is alkyl which is unsubstituted or substituted with R¹⁶ or NHC(O)NHR¹⁶;

R¹⁶ is phenyl;

B¹ is H or R¹⁹;

R¹⁹ is R²¹, R²² or R²³;

R²¹ is heteroaryl;

R²² is heterocycloalkyl;

R²³ is alkynyl, which is unsubstituted or substituted with R²⁴, OR²⁴ or N(R²⁴)₂;

R²⁴ is alkyl;

C¹ is S or N(C²);

C² is R³⁰;

R³⁰ is cycloalkyl;

D¹ is N, CH or C(D²);

D² is R³⁷;

R³⁷ is heteroaryl;

wherein each foregoing cyclic moiety is independently unsubstituted or substituted or further unsubstituted or further substituted with one or two or three or four of independently selected R⁴⁴, OR⁴⁴, CN, CF₃, F, Cl, Br or I;

R⁴⁴ is R⁴⁷ or R⁴⁸;

R⁴⁷ is heterocycloalkyl;

R⁴⁸ is alkyl substituted with OP(O)(OH)₂;

wherein R⁴⁷ is unsubstituted or substituted with alkyl.

Still another embodiment pertains to compositions comprising an excipient and a therapeutically effective amount of a compound having formula (I).

Still another embodiment pertains to methods of treating diseases involving overexpression or unregulation of Protein kinases in a mammal, the methods comprising administering thereto a therapeutically effective amount of a compound having formula (I).

Still another embodiment pertains to methods of treating cancer in a mammal comprising administering thereto a therapeutically effective amount of a compound having formula (I).

Still another embodiment pertains to methods of treating bladder cancer, breast cancer, cervical cancer, colon cancer, endometrial cancer, esophageal cancer, lung cancer, ovarian cancer, pancreatic cancer, prostate cancer, rectal cancer, skin cancer, stomach cancer and thyroid cancer in a mammal, the methods comprising administering thereto a therapeutically effective amount of a compound having formula (I).

Still another embodiment pertains to compositions comprising an excipient and a therapeutically effective amount of a compound having formula (I) and a therapeutically effective amount of one additional therapeutic agent or more than one additional therapeutic agent.

Still another embodiment pertains to methods of treating diseases involving overexpression or unregulation of protein kinases in a mammal, the methods comprising administering thereto a therapeutically effective amount of a compound having formula (I) and a therapeutically effective amount of one additional therapeutic agent or more than one additional therapeutic agent, with or without radiation.

Still another embodiment pertains to methods of treating bladder cancer, breast cancer, cervical cancer, colon cancer, endometrial cancer, esophageal cancer, leukemia, lymphoma, lung cancer, ovarian cancer, pancreatic cancer, prostate cancer, rectal cancer, skin cancer, stomach cancer or thyroid cancer in a mammal, the methods comprising administering thereto a therapeutically effective amount of a compound having formula (I) and a therapeutically effective amount of one additional therapeutic agent or more than one additional therapeutic agent.

Still another embodiment pertains to

4-amino-N-(4-((3-toluidinocarbonyl)amino)phenyl)thieno[2,3-d]pyrimidine-5-carboxamide,

4-amino-N-(4-(((3-fluoroanilino)carbonyl)amino)phenyl)thieno[2,3-d]pyrimidine-5-carboxamide,

4-amino-N-(4-(((3-fluoro-4-methylanilino)carbonyl)amino)phenyl)thieno[2,3-d]pyrimidine-5-carboxamide,

4-amino-N-(4-((4-toluidinocarbonyl)amino)phenyl)thieno[2,3-d]pyrimidine-5-carboxamide,

4-amino-N-(4-(((4-fluoroanilino)carbonyl)amino)phenyl)thieno[2,3-d]pyrimidine-5-carboxamide,

4-amino-N-(4-(((3-chloro-4-fluoroanilino)carbonyl)amino)phenyl)thieno[2,3-d]pyrimidine-5-carboxamide,

4-amino-N-(4-(((3-ethylanilino)carbonyl)amino)phenyl)thieno[2,3-d]pyrimidine-5-carboxamide,

4-amino-N-(4-(((3-chloroanilino)carbonyl)amino)phenyl)thieno[2,3-d]pyrimidine-5-carboxamide,

4-amino-N-(4-(((3-cyanoanilino)carbonyl)amino)phenyl)thieno[2,3-d]pyrimidine-5-carboxamide,

4-amino-N-(4-(((2-fluoroanilino)carbonyl)amino)phenyl)thieno[2,3-d]pyrimidine-5-carboxamide,

4-amino-N-(4-(((3-(trifluoromethyl)anilino)carbonyl)amino)phenyl)thieno[2,3-d]pyrimidine-5-carboxamide,

4-amino-N-(4-(((4-fluoro-3-(trifluoromethyl)anilino)carbonyl)amino)phenyl)thieno[2,3-d]pyrimidine-5-carboxamide,

4-amino-N-(4-(((3-methoxyanilino)carbonyl)amino)phenyl)thieno[2,3-d]pyrimidine-5-carboxamide,

4-amino-N-(4-(((5-fluoro-2-methylanilino)carbonyl)amino)phenyl)thieno[2,3-d]pyrimidine-5-carboxamide,

4-amino-N-(4-(((4-(trifluoromethyl)anilino)carbonyl)amino)phenyl)thieno[2,3-d]pyrimidine-5-carboxamide,

4-amino-N-(4-(((2-fluoro-5-methylanilino)carbonyl)amino)phenyl)thieno[2,3-d]pyrimidine-5-carboxamide,

4-amino-N-(4-((2-toluidinocarbonyl)amino)phenyl)thieno[2,3-d]pyrimidine-5-carboxamide,

4-amino-N-(4-(((4-methoxyanilino)carbonyl)amino)phenyl)thieno[2,3-d]pyrimidine-5-carboxamide,

4-amino-N-(4-(((3,5-dimethylanilino)carbonyl)amino)phenyl)thieno[2,3-d]pyrimidine-5-carboxamide,

4-amino-N-(4-(((2-fluoro-5-(trifluoromethyl)anilino)carbonyl)amino)phenyl)thieno[2,3-d]pyrimidine-5-carboxamide,

4-amino-N-(4-((anilinocarbonyl)amino)phenyl)thieno[2,3-d]pyrimidine-5-carboxamide,

4-amino-N-(4-(((3-chloroanilino)carbonyl)amino)phenyl)-6-(1-methyl-1H-pyrazol-4-yl)thieno[2,3-d]pyrimidine-5-carboxamide,

4-amino-6-(1-methyl-1H-pyrazol-4-yl)-N-(4-((3-toluidinocarbonyl)amino)phenyl)thieno[2,3-d]pyrimidine-5-carboxamide

4-amino-N-(4-(((2-fluoro-5-methylanilino)carbonyl)amino)phenyl)-6-(1-methyl-1H-pyrazol-4-yl)thieno[2,3-d]pyrimidine-5-carboxamide,

4-amino-N-(4-(((4-fluoro-3-(trifluoromethyl)anilino)carbonyl)amino)phenyl)-6-(1-methyl-1H-pyrazol-4-yl)thieno[2,3-d]pyrimidine-5-carboxamide,

4-amino-N-(4-(((2-fluoro-5-(trifluoromethyl)anilino)carbonyl)amino)phenyl)-6-(1-methyl-1H-pyrazol-4-yl)thieno[2,3-d]pyrimidine-5-carboxamide,

4-amino-6-(1-methyl-1H-pyrazol-4-yl)-N-(4-(((4-(trifluoromethyl)anilino)carbonyl)amino)phenyl)thieno[2,3-d]pyrimidine-5-carboxamide,

4-amino-6-(1-methyl-1H-pyrazol-4-yl)-N-(4-phenoxyphenyl)thieno[2,3-d]pyrimidine-5-carboxamide,

4-amino-N-(4-phenoxyphenyl)thieno[2,3-d]pyrimidine-5-carboxamide,

4-amino-N-(4-(3-methylphenoxy)phenyl)-6-(1-methyl-1H-pyrazol-4-yl)thieno[2,3-d]pyrimidine-5-carboxamide,

4-amino-N-(4-(4-chlorophenoxy)phenyl)-6-(1-methyl-1H-pyrazol-4-yl)thieno[2,3-d]pyrimidine-5-carboxamide,

4-amino-N-(4-(4-methylphenoxy)phenyl)-6-(1-methyl-1H-pyrazol-4-yl)thieno[2,3-d]pyrimidine-5-carboxamide,

4-amino-N-(4-(3-chlorophenoxy)phenyl)-6-(1-methyl-1H-pyrazol-4-yl)thieno[2,3-d]pyrimidine-5-carboxamide,

4-amino-6-(1-methyl-1H-pyrazol-4-yl)-N-(4-(phenylsulfanyl)phenyl)thieno[2,3-d]pyrimidine-5-carboxamide,

4-amino-N-(4-(4-methylphenoxy)phenyl)thieno[2,3-d]pyrimidine-5-carboxamide,

4-amino-N-(4-phenoxyphenyl)-6-(1H-pyrazol-4-yl)thieno[2,3-d]pyrimidine-5-carboxamide,

4-amino-N-(4-phenoxyphenyl)-6-(3-thienyl)thieno[2,3-d]pyrimidine-5-carboxamide,

4-amino-6-(4-methyl-1-piperazinyl)-N-(4-((3-toluidinocarbonyl)amino)phenyl)thieno[2,3-d]pyrimidine-5-carboxamide,

4-amino-N-methyl-N-(4-((3-toluidinocarbonyl)amino)phenyl)thieno[2,3-d]pyrimidine-5-carboxamide,

4-amino-N-(4-phenoxyphenyl)thieno[3,2-c]pyridine-3-carboxamide,

4-amino-N-(3-phenoxyphenyl)thieno[3,2-c]pyridine-3-carboxamide,

4-amino-N-(4-benzylphenyl)thieno [3,2-c]pyridine-3-carboxamide,

4-amino-N-(4-((3-toluidinocarbonyl)amino)phenyl)thieno[3,2-c]pyridine-3-carboxamide,

4-amino-N-(4-(benzoylamino)phenyl)thieno[3,2-c]pyridine-3-carboxamide,

4-amino-7-(1-methyl-1H-pyrazol-4-yl)-N-(4-((3-toluidinocarbonyl)amino)phenyl)thieno[3,2-c]pyridine-3-carboxamide,

4-amino-N-(4-(benzoylamino)phenyl)-7-(1-methyl-1H-pyrazol-4-yl)thieno[3,2-c]pyridine-3-carboxamide,

4-amino-7-(1-methyl-1H-pyrazol-4-yl)-N-(4-phenoxyphenyl)thieno[3,2-c]pyridine-3-carboxamide,

4-amino-7-(1-methyl-1H-pyrazol-4-yl)-N-(3-phenoxyphenyl)thieno[3,2-c]pyridine-3-carboxamide,

4-amino-N-(4-benzylphenyl)-7-(1-methyl-1H-pyrazol-4-yl)thieno[3,2-c]pyridine-3-carboxamide,

4-amino-7-(4-(4-methyl-1-piperazinyl)cyclohexyl)-N-(4-phenoxyphenyl)-7H-pyrrolo[2,3-d]pyrimidine-5-carboxamide,

4-amino-7-(4-(4-methyl-1-piperazinyl)cyclohexyl)-N-(3-phenoxyphenyl)-7H-pyrrolo[2,3-d]pyrimidine-5-carboxamide,

4-amino-7-(4-(4-methyl-1-piperazinyl)cyclohexyl)-N-(4-((3-toluidinocarbonyl)amino)phenyl)-7H-pyrrolo[2,3-d]pyrimidine-5-carboxamide,

4-amino-N-(4-((4-aminophenyl)sulfanyl)phenyl)-7-(4-(4-methyl-1-piperazinyl)cyclohexyl)-7H-pyrrolo[2,3-d]pyrimidine-5-carboxamide,

4-amino-N-(4-benzylphenyl)-7-(4-(4-methyl-1-piperazinyl)cyclohexyl)-7H-pyrrolo[2,3-d]pyrimidine-5-carboxamide,

4-amino-7-(4-(4-methyl-1-piperazinyl)cyclohexyl)-N-(4-(phenylsulfonyl)phenyl)-7H-pyrrolo[2,3-d]pyrimidine-5-carboxamide,

4-amino-1-(4-(4-morpholinyl)cyclohexyl)-1H-pyrazolo(3,4-d)pyrimidine-3-carboxylic acid,

4-amino-1-(4-(4-morpholinyl)cyclohexyl)-N-(4-phenoxyphenyl)-1H-pyrazolo(3,4-d)pyrimidine-3-carboxamide,

N-(4-((E)-2-(4-amino-7-(1-methyl-1H-pyrazol-4-yl)thieno[3,2-c]pyridin-3-yl)ethenyl)phenyl)-N′-(3-methylphenyl)urea

7-(1-methyl-1H-pyrazol-4-yl)-3-((E)-2-(4-phenoxyphenyl)ethenyl)thieno[3,2-c]pyridin-4-amine,

3-((E)-2-(1,1′-biphenyl)-4-ylethenyl)-7-( i-methyl-1H-pyrazol-4-yl)thieno[3,2-c]pyridin-4-amine,

4-amino-N-(4-((anilinocarbonyl)amino)phenyl)-7-(1-methyl-1H-pyrazol-4-yl)thieno[3,2-c]pyridine -3-carboxamide,

4-amino-N-(4-((((3-fluorophenyl)amino)carbonyl)amino)phenyl)-7-(1-methyl-1H-pyrazol-4-yl)thieno[3,2-c]pyridine-3-carboxamide,

4-amino-N-(4-(((cyclohexylamino)carbonyl)amino)phenyl)-7-(1-methyl-1H-pyrazol-4-yl)thieno[3,2-c]pyridine-3-carboxamide,

4-amino-N-(4-((((4-methylphenyl)amino)carbonyl)amino)phenyl)-7-(1-methyl-1H-pyrazol-4-yl)thieno[3,2-c]pyridine-3-carboxamide,

4-amino-N-(4-((((2-methylphenyl)amino)carbonyl)amino)phenyl)-7-(1-methyl-1H-pyrazol-4-yl)thieno[3,2-c]pyridine-3-carboxamide,

4-amino-N-(3-((anilinocarbonyl)amino)phenyl)-7-(1-methyl-1H-pyrazol-4-yl)thieno[3,2-c]pyridine-3-carboxamide,

4-amino-N-(3-((((2-methylphenyl)amino)carbonyl)amino)phenyl)-7-(1-methyl-1H-pyrazol-4-yl)thieno[3,2-c]pyridine-3-carboxamide,

4-amino-N-(3-((((4-methylphenyl)amino)carbonyl)amino)phenyl)-7-(1-methyl-1H-pyrazol-4-yl)thieno[3,2-c]pyridine-3-carboxamide,

4-amino-N-(3-((((3-methylphenyl)amino)carbonyl)amino)phenyl)-7-(1-methyl-1H-pyrazol-4-yl)thieno[3,2-c]pyridine-3-carboxamide,

4-amino-N-(3-(benzoylamino)phenyl)-7-(1-methyl-1H-pyrazol-4-yl)thieno[3,2-c]pyridine-3-carboxamide,

4-amino-N-(1-(anilinocarbonyl)piperidin-4-yl)-7-(1-methyl-1H-pyrazol-4-yl)thieno[3,2-c]pyridine-3-carboxamide,

4-amino-N-(1-benzoylpiperidin-4-yl)-7-(1-methyl-1H-pyrazol-4-yl)thieno[3,2-c]pyridine-3-carboxamide,

trans4-amino-N-(4-(benzoylamino)cyclohexyl)-7-(1-methyl-1H-pyrazol-4-yl)thieno[3,2-c]pyridine-3-carboxamide,

trans4-amino-N-(4-((anilinocarbonyl)amino)cyclohexyl)-7-(1-methyl-1H-pyrazol-4-yl)thieno[3,2-c]pyridine-3-carboxamide,

trans4-amino-N-(4-((((2-fluorophenyl)amino)carbonyl)amino)cyclohexyl)-7-(1-methyl-1H-pyrazol-4-yl)thieno[3,2-c]pyridine-3-carboxamide,

4-amino-N-(4-((anilinocarbonyl)amino)benzyl)-7-(1-methyl-1H-pyrazol-4-yl)thieno[3,2-c]pyridine-3-carboxamide,

4-amino-N-(3-((anilinocarbonyl)amino)benzyl)-7-(1-methyl-1H-pyrazol-4-yl)thieno[3,2-c]pyridine-3-carboxamide,

4-amino-N-((1-(anilinocarbonyl)piperidin-4-yl)methyl)-7-(1-methyl-1H-pyrazol-4-yl)thieno[3,2-c]pyridine-3-carboxamide,

4-amino-N-(4-(((anilinocarbonyl)amino)methyl)phenyl)-7-(1-methyl-1H-pyrazol-4-yl)thieno[3,2-c]pyridine-3-carboxamide,

4-amino-N-(3-(((anilinocarbonyl)amino)methyl)phenyl)-7-(1-methyl-1H-pyrazol-4-yl)thieno[3,2-c]pyridine-3-carboxamide,

cis-4-amino-N-(4-((anilinocarbonyl)amino)cyclohexyl)-7-(1-methyl-1H-pyrazol-4-yl)thieno[3,2-c]pyridine-3-carboxamide,

cis-4-amino-N-((1S,3R)-3-((anilinocarbonyl)amino)cyclohexyl)-7-(1-methyl-1H-pyrazol-4-yl)thieno[3,2-c]pyridine-3-carboxamide,

cis-4-amino-N-((1S,3R)-3-(anilinocarbonyl)cyclohexyl)-7-(1-methyl-1H-pyrazol-4-yl)thieno[3,2-c]pyridine-3-carboxamide,

4-amino-N-(3-(((anilinocarbonyl)amino)methyl)phenyl)-7-(1-(2-hydroxyethyl)-1H-pyrazol-4-yl)thieno[3,2-c]pyridine-3-carboxamide,

2-(4-(4-amino-3-(((3-(((anilinocarbonyl)amino)methyl)phenyl)amino)carbonyl)thieno[3,2-c]pyridin-7-yl)-1H-pyrazol-1-yl)ethyl dihydrogen phosphate,

4-amino-N-(4-((((2-fluoro-3-(trifluoromethyl)phenyl)amino)carbonyl)amino)phenyl)-6-(1-methyl-1H-pyrazol-4-yl)thieno[2,3-d]pyrimidine-5-carboxamide,

4-amino-N-(4-((anilinocarbonyl)amino)phenyl)-6-(1-methyl-1H-pyrazol-4-yl)thieno[2,3-d]pyrimidine-5-carboxamide,

4-amino-N-(4-((anilinocarbonyl)amino)phenyl)-6-thien-3-ylthieno[2,3-d]pyrimidine-5-carboxamide,

4-amino-N-(4-((anilinocarbonyl)amino)phenyl)-6-morpholin-4-ylthieno[2,3-d]pyrimidine-5-carboxamide,

4-amino-N-(4-(((thien-3-ylamino)carbonyl)amino)phenyl)thieno[2,3-d]pyrimidine-5-carboxamide,

4-amino-N-(4-(((cyclopentylamino)carbonyl)amino)phenyl)thieno[2,3-d]pyrimidine-5-carboxamide,

4-amino-N-(4-(((pyridin-3-ylamino)carbonyl)amino)phenyl)thieno[2,3-d]pyrimidine-5-carboxamide,

4-amino-N-(4-((((5-methylisoxazol-3-yl)amino)carbonyl)amino)phenyl)thieno[2,3-d]pyrimidine-5-carboxamide,

4-amino-N-(4-(((cyclopropylamino)carbonyl)amino)phenyl)thieno[2,3-d]pyrimidine-5-carboxamide,

4-amino-N-(4-((((2,4-difluorophenyl)amino)carbonyl)amino)phenyl)thieno[2,3-d]pyrimidine-5-carboxamide,

4-amino-N-(4-((((3,4-difluorophenyl)amino)carbonyl)amino)phenyl)thieno[2,3-d]pyrimidine-5-carboxamide,

4-amino-N-(4-((((3-(morpholin-4-ylmethyl)phenyl)amino)carbonyl)amino)phenyl)thieno[2,3-d]pyrimidine-5-carboxamide,

4-amino-N-(4-((anilinocarbonyl)amino)cyclohexyl)thieno[2,3-d]pyrimidine-5-carboxamide,

4-amino-N-(4-((((3,5-dimethylisoxazol-4-yl)amino)carbonyl)amino)phenyl)thieno[2,3-d]pyrimidine-5-carboxamide,

4-amino-N-(4-(((1,3-thiazol-2-ylamino)carbonyl)amino)phenyl)thieno[2,3-d]pyrimidine-5-carboxamide,

4-amino-N-(4-(((isoxazol-3-ylamino)carbonyl)amino)phenyl)thieno[2,3-d]pyrimidine-5-carboxamide,

4-amino-N-(1-(anilinocarbonyl)piperidin-4-yl)thieno[2,3-d]pyrimidine-5-carboxamide,

4-amino-N-(3-((anilinocarbonyl)amino)phenyl)thieno[2,3-d]pyrimidine-5-carboxamide,

4-amino-N-(4-((anilinocarbonyl)amino)phenyl)-6-(3-methoxyprop-1-ynyl)thieno[2,3-d]pyrimidine-5-carboxamide,

4-amino-N-(4-((anilinocarbonyl)amino)phenyl)-6-ethynylthieno[2,3-d]pyrimidine-5-carboxamide,

4-amino-N-(4-((anilinocarbonyl)amino)phenyl)-6-(thien-3-ylethynyl)thieno[2,3-d]pyrimidine-5-carboxamide,

4-amino-N-(4-((anilinocarbonyl)amino)phenyl)-6-(3-(dimethylamino)prop-1-ynyl)thieno[2,3-d]pyrimidine-5-carboxamide,

4-amino-N-(4-((2-fluorobenzoyl)amino)phenyl)thieno[2,3-d]pyrimidine-5-carboxamide,

4-amino -N-(4-((3-fluorobenzoyl)amino)phenyl)thieno [2,3-d]pyrimidine-5-carboxamide,

4-amino-N-(4-((4-fluorobenzoyl)amino)phenyl)thieno[2,3-d]pyrimidine-5-carboxamide,

4-amino-N-(4-((2-methylbenzoyl)amino)phenyl)thieno[2,3-d]pyrimidine-5-carboxamide,

4-amino-N-(4-((3-methylbenzoyl)amino)phenyl)thieno[2,3-d]pyrimidine-5-carboxamide,

4-amino-N-(4-((((3-(hydroxymethyl)phenyl)amino)carbonyl)amino)phenyl)thieno[2,3-d]pyrimidine-5-carboxamide,

4-amino-N-(3-(((anilinocarbonyl)amino)methyl)phenyl)thieno[2,3-d]pyrimidine-5-carboxamide,

4-amino-N-(3-((((2-methylphenyl)amino)carbonyl)amino)phenyl)thieno[2,3-d]pyrimidine-5-carboxamide,

4-amino-N-(3-(((((3-methylphenyl)amino)carbonyl)amino)methyl)phenyl)thieno[2,3-d]pyrimidine-5-carboxamide,

4-amino-N-(3-(((((3-fluorophenyl)amino)carbonyl)amino)methyl)phenyl)thieno[2,3-d]pyrimidine-5-carboxamide,

4-amino-N-(3-((((3-methylphenyl)amino)carbonyl)amino)phenyl)thieno[2,3-d]pyrimidine-5-carboxamide,

4-amino-N-(3-((((4-methylphenyl)amino)carbonyl)amino)phenyl)thieno[2,3-d]pyrimidine-5-carboxamide,

4-amino-N-(3-((((2-fluorophenyl)amino)carbonyl)amino)phenyl)thieno[2,3-d]pyrimidine-5-carboxamide,

4-amino-N-(3-((((3-fluorophenyl)amino)carbonyl)amino)phenyl)thieno[2,3-d]pyrimidine-5-carboxamide,

4-amino-N-(3-((((4-fluorophenyl)amino)carbonyl)amino)phenyl)thieno [2,3-d]pyrimidine-5-carboxamide,

4-amino-N-(3-(((((3-(trifluoromethyl)phenyl)amino)carbonyl)amino)methyl)phenyl)thieno[2,3-d]pyrimidine-5-carboxamide,

4-amino-N-(5-(2-((3-fluorophenyl)amino)-2-oxoethyl)-1,3-thiazol-2-yl)thieno[2,3-d]pyrimidine-5-carboxamide,

N-(4-(2-(4-aminothieno[2,3-d]pyrimidin-5-yl)ethyl)phenyl)-N′-phenylurea,

4-amino-N-(4-((((3-(3-hydroxypropoxy)phenyl)amino)carbonyl)amino)phenyl)thieno[2,3-d]pyrimidine-5-carboxamide,

4-amino-N-(4-((anilinocarbonyl)amino)benzyl)thieno[2,3-d]pyrimidine-5-carboxamide,

4-amino-N-(4-((((3-methylphenyl)amino)carbonyl)amino)benzyl)thieno[2,3-d]pyrimidine-5-carboxamide,

4-amino-N-(4-((((3-fluorophenyl)amino)carbonyl)amino)benzyl)thieno[2,3-d]pyrimidine-5-carboxamide

and therapeutically acceptable salts, prodrugs, salts of prodrugs and metabolites thereof.

### DETAILED DESCRIPTION OF THE INVENTION

Variable moieties of compounds herein are represented by identifiers (capital letters with numerical and/or alphabetical superscripts) and may be specifically embodied.

It is meant to be understood that proper valences are maintained for all moieties and combinations thereof, that monovalent moieties having more than one atom are attached through their left ends.

It is also meant to be understood that a specific embodiment of a variable moiety may be the same or different as another specific embodiment having the same identifier.

The term “cyclic moiety,” as used herein, means benzene, cycloalkane, cycloalkyl, cycloalkene, cycloalkenyl, heteroarene, heteroaryl, heterocycloalkane, heterocycloalkyl, heterocycloalkene, heterocycloalkenyl, phenyl, spiroalkyl, spiroalkenyl, spiroheteroalkyl and spiroheteroalkenyl.

The term “cycloalkane,” as used herein, means C₃-cycloalkane, C₄-cycloalkane, C₅-cycloalkane and C₆-cycloalkane.

The term “cycloalkyl,” as used herein, means C₃-cycloalkyl, C₄-cycloalkyl, C₅-cycloalkyl and C₆-cycloalkyl.

The term “cycloalkene,” as used herein, means C₄-cycloalkene, C₅-cycloalkene and C₆-cycloalkene.

The term “cycloalkenyl,” as used herein, means C₄-cycloalkenyl, C₅-cycloalkenyl and C₆-cycloalkenyl.

The term “heteroarene,” as used herein, means furan, imidazole, isothiazole, isoxazole, 1,2,3-oxadiazole, 1,2,5-oxadiazole, oxazole, pyrazine, pyrazole, pyridazine, pyridine, pyrimidine, pyrrole, thiazole, thiophene, triazine and 1,2,3-triazole.

The term “heteroaryl,” as used herein, means furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl and 1,2,3-triazolyl.

The term “heterocycloalkane,” as used herein, means cycloalkane having one or two or three CH₂ moieties replaced with independently selected O, S, S(O), SO₂ or NH and one or two CH moieties unreplaced or replaced with N and also means cycloalkane having one or two or three CH₂ moieties unreplaced or replaced with independently selected O, S, S(O), SO₂ or NH and one or two CH moieties replaced with N.

The term “heterocycloalkyl,” as used herein, means cycloalkyl having one or two or three CH₂ moieties replaced with independently selected O, S, S(O), SO₂ or NH and one or two CH moieties unreplaced or replaced with N and also means cycloalkyl having one or two or three CH₂ moieties unreplaced or replaced with independently selected O, S, S(O), SO₂ or NH and one or two CH moieties replaced with N.

The term “heterocycloalkene,” as used herein, means cycloalkene having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected O, S, S(O), SO₂ or NH and one or two CH moieties replaced with N.

The term “heterocycloalkenyl,” as used herein, means cycloalkenyl having one or two or three CH₂ moieties replaced with independently selected O, S, S(O), SO₂ or NH and one or two CH moieties unreplaced or replaced with N and also means cycloalkenyl having one or two or three CH₂ moieties unreplaced or replaced with independently selected O, S, S(O), SO₂ or NH and one or two CH moieties replaced with N.

The term “alkenyl,” as used herein, means C₂-alkenyl, C₃-alkenyl, C₄-alkenyl, C₅-alkenyl and C₆-alkenyl.

The term “alkyl,” as used herein, means C₁-alkyl, C₂-alkyl, C₃-alkyl, C₄-alkyl, C₅-alkyl and C₆-alkyl.

The term “alkynyl,” as used herein, means C₂-alkynyl, C₃-alkynyl, C₄-alkynyl, C₅-alkynyl and C₆-alkynyl.

The term “C₂-alkenyl,” as used herein, means ethenyl (vinyl).

The term “C₃-alkenyl,” as used herein, means 1-propen-1-yl, 1-propen-2-yl (isopropenyl) and 1-propen-3-yl (allyl).

The term “C₄-alkenyl,” as used herein, means 1-buten-1-yl, 1-buten-2-yl, 1,3-butadien-1-yl, 1,3-butadien-2-yl, 2-buten-1-yl, 2-buten-2-yl, 3-buten-1-yl, 3-buten-2-yl, 2-methyl-1-propen-1-yl and 2-methyl-2-propen-1-yl.

The term “C₅-alkenyl,” as used herein, means 2-methylene-3-buten-1-yl, 2-methylenebut-1-yl, 2-methyl-1-buten-1-yl, 2-methyl-1,3-butadien-1-yl, 2-methyl-2-buten-1-yl, 2-methyl-3-buten-1-yl, 2-methyl-3-buten-2-yl, 3-methyl-1-buten-1-yl, 3-methyl-1-buten-2-yl, 3-methyl-1,3-butadien-1-yl, 3-methyl-1,3-butadien-2-yl, 3-methyl-2-buten-1-yl, 3-methyl-2-buten-2-yl, 3-methyl-3-buten-1-yl, 3-methyl-3-buten-2-yl, 1-penten-1-yl, 1-penten-2-yl, 1-penten-3-yl, 1,3-pentadien-1-yl, 1,3-penta-dien-2-yl, 1,3-pentadien-3-yl, 1,4-pentadien-1-yl, 1,4-pentadien-2-yl, 1,4-pentadien-3-yl, 2-penten-1-yl, 2-penten-2-yl, 2-penten-3-yl, 2,4-pentadien-1-yl, 2,4-pentadien-2-yl, 3-penten-1-yl, 3-penten-2-yl, 4-penten-1-yl and 4-penten-2-yl.

The term “C₆-alkenyl,” as used herein, means 2,2-dimethyl-3-buten-1-yl, 2,3-dimethyl-1-buten-1-yl, 2,3-dimethyl-1,3-butadien-1-yl, 2,3-dimethyl-2-buten-1-yl, 2,3-dimethyl-3-buten-1-yl, 2,3-dimethyl-3-buten-2-yl, 3,3-dimethyl-1-buten-1-yl, 3,3-dimethyl-1-buten-2-yl, 2-ethenyl-1,3-butadien-1-yl, 2-ethenyl-2-buten-1-yl, 2-ethyl-1-buten-1-yl, 2-ethyl-1,3-butadien-1-yl, 2-ethyl-2-buten-1-yl, 2-ethyl-3-buten-1-yl, 1-hexen-1-yl, 1-hexen-2-yl, 1-hexen-3-yl, 1,3-hexadien-1-yl, 1,3-hexadien-2-yl, 1,3-hexadien-3-yl, 1,3,5-hexatrien-1-yl, 1,3,5-hexatrien-2-yl, 1,3,5-hexatrien-3-yl, 1,4-hexadien-1-yl, 1,4-hexadien-2-yl, 1,4-hexadien-3-yl, 1,5-hexadien-1-yl, 1,5-hexadien-2-yl, 1,5-hexadien-3-yl, 2-hexen-1-yl, 2-hexen-2-yl, 2-hexen-3-yl, 2,4-hexadien-1-yl, 2,4-hexadien-2-yl, 2,4-hexadien-3-yl, 2,5-hexadien-1-yl, 2,5-hexadien-2-yl, 2,5-hexadien-3-yl, 3-hexen-1-yl, 3-hexen-2-yl, 3-hexen-3-yl, 3,5-hexadien-1-yl, 3,5-hexadien-2-yl, 3,5-hexadien-3-yl, 4-hexen-1-yl, 4-hexen-2-yl, 4-hexen-3-yl, 5-hexen-1-yl, 5-hexen-2-yl, 5-hexen-3-yl, 2-methylene-3-methyl-3-buten-1-yl, 2-methylene-3-methylbut-1-yl, 2-methylene-3-penten-1-yl, 2-methylene-4-penten-1-yl, 2-methylenepent-1-yl, 2-methylenepent-3-yl, 3-methylene-1-penten-1-yl, 3-methylene-1-penten-2-yl, 3-methylenepent-1-yl, 3-methylene-1,4-pentadien-1-yl, 3-methylene-1,4-pentadien-2-yl, 3-methylene-pent-2-yl, 2-methyl-1-penten-1-yl, 2-methyl-1-penten-3-yl, 2-methyl-1,3-pentadien-1-yl, 2-methyl-1,3-pentadien-3-yl, 2-methyl-1,4-pentadien-1-yl, 2-methyl-1,4-pentadien-3-yl, 2-methyl-2-penten-1-yl, 2-methyl-2-penten-3-yl, 2-methyl-2,4-pentadien-1-yl, 2-methyl-2,4-pentadien-3-yl, 2-methyl-3-penten-1-yl, 2-methyl-3-penten-2-yl, 2-methyl-3-penten-3-yl, 2-methyl-4-penten-1-yl, 2-methyl-4-penten-2-yl, 2-methyl-4-penten-3-yl, 3-methyl-1-penten-1-yl, 3-methyl-1-penten-2-yl, 3-methyl-1,3-pentadien-1-yl, 3-methyl-1,3-pentadien-2-yl, 3-methyl-1,4-pentadien-1-yl, 3-methyl-1,4-pentadien-2-yl, 3-methyl-2-penten-1-yl, 3-methyl-2-penten-2-yl, 3-methyl-2,4-pentadien-1-yl, 3-methyl-3-penten-1yl, 3-methyl-3-penten-2-yl, 3-methyl-4-penten-1-yl, 3-methyl-4-penten-2-yl, 3-methyl-4-penten-3-yl, 4-methyl-1-penten-1-yl, 4-methyl-1-penten-2-yl, 4-methyl-1-penten-3-yl, 4-methyl-1,3-pentadien-1-yl, 4-methyl-1,3-pentadien-2-yl, 4-methyl-1,3-pentadien-3-yl, 4-methyl-1,4-pentadien-1-yl, 4-methyl-1,4-pentadien-2-yl, 4-methyl-1,4-pentadien-3-yl, 4-methylene-2-penten-3-yl, 4-methyl-2-penten-1-yl, 4-methyl-2-penten-2-yl, 4-methyl-2-penten-3-yl, 4-methyl-2,4-pentadien-1-yl, 4-methyl-2,4-pentadien-2-yl, 4-methyl-3-penten-1-yl, 4-methyl-3-penten-2-yl, 4-methyl-3-penten-3-yl, 4-methyl-4-penten-1-yl and 4-methyl-4-penten-2-yl,

The term “C₁-alkyl,” as used herein, means methyl.

The term “C₂-alkyl,” as used herein, means ethyl.

The term “C₃-alkyl,” as used herein, means prop-1-yl and prop-2-yl (isopropyl).

The term “C₄-alkyl,” as used herein, means but-1-yl, but-2-yl, 2-methylprop-1-yl and 2-methylprop-2-yl (tert-butyl).

The term “C₅-alkyl,” as used herein, means 2,2-dimethylprop-1-yl (neo-pentyl), 2-methylbut-1-yl, 2-methylbut-2-yl, 3-methylbut-1-yl, 3-methylbut-2-yl, pent-1-yl, pent-2-yl and pent-3-yl.

The term “C₆-alkyl,” as used herein, means 2,2-dimethylbut-1-yl, 2,3-dimethylbut-1-yl, 2,3-dimethylbut-2-yl, 3,3-dimethylbut-1-yl, 3,3-dimethylbut-2-yl, 2-ethylbut-1-yl, hex-1-yl, hex-2-yl, hex-3-yl, 2-methylpent-1-yl, 2-methylpent-2-yl, 2-methylpent-3-yl, 3-methylpent-1-yl, 3-methylpent-2-yl, 3-methylpent-3-yl, 4-methylpent-1-yl and 4-methylpent-2-yl.

The term “C₂-alkynyl,” as used herein, means ethynyl (acetylenyl).

The term “C₃-alkynyl,” as used herein, means 1-propyn-1-yl and 2-propyn-1-yl (propargyl).

The term “C₄-alkynyl,” as used herein, means 1-butyn-1-yl, 1,3-butadiyn-1-yl, 2-butyn-1-yl, 3-butyn-1-yl and 3-butyn-2-yl.

The term “C₅-alkynyl,” as used herein, means 2-methyl-3-butyn-1-yl, 2-methyl-3-butyn-2-yl, 3-methyl-1-butyn-1-yl, 1,3-pentadiyn-1-yl, 1,4-pentadiyn-1-yl, 1,4-pentadiyn-3-yl, 2,4-pentadiyn-1-yl, 1-pentyn-1-yl, 1-pentyn-3-yl, 2-pentyn-1-yl, 3-pentyn-1-yl, 3-pentyn-2-yl, 4-pentyn-1-yl and 4-pentyn-2-yl.

The term “C₆-alkynyl,” as used herein, means 2,2-dimethyl-3-butyn-1-yl, 3,3-dimethyl-1-butyn-1-yl, 2-ethyl-3-butyn-1-yl, 2-ethynyl-3-butyn-1-yl, 1-hexyn-1-yl, 1-hexyn-3-yl, 1,3-hexadiyn-1-yl, 1,3,5-hexatriyn-1-yl, 1,4-hexadiyn-1-yl, 1,4-hexadiyn-1-yl, 1,4-hexadiyn-3-yl, 1,5-hexadiyn-1-yl, 1,5-hexadiyn-3-yl, 2-hexyn-1-yl, 2,5-hexadiyn-1-yl, 3-hexyn-1-yl, 3-hexyn-2-yl, 3,5-hexadiyn-2-yl, 4-hexyn-1-yl, 4-hexyn-2-yl, 4-hexyn-3-yl, 5-hexyn-1-yl, 5-hexyn-2-yl, 5-hexyn-3-yl, 2-methyl-3-pentyn-1-yl, 2-methyl-3-pentyn-2-yl, 2-methyl-4-pentyn-1-yl, 2-methyl-4-pentyn-2-yl, 2-methyl-4-pentyn-3-yl, 3-methyl-1-pentyn-1-yl, 3-methyl-4-pentyn-1-yl, 3-methyl-4-pentyn-2-yl, 3-methyl-1,4-pentadiyn-1-yl, 3-methyl-1,4-pentadiyn-3-yl, 3-methyl-4-pentyn-1-yl, 3-methyl-4-pentyn-3-yl, 4-methyl-1-pentyn-1-yl and 4-methyl-2-pentyn-1-yl.

The term “C₄-cycloalkane,” as used herein, means cyclobutane.

The term “C₅-cycloalkane,” as used herein, means cyclopentane.

The term “C₆-cycloalkane,” as used herein, means cyclohexane.

The term “C₄-cycloalkene,” as used herein, means cyclobutene and 1,3-cyclobutadiene.

The term “C₅-cycloalkene,” as used herein, means cyclopentene and 1,3-cyclopentadiene.

The term “C₆-cycloalkene,” as used herein, means cyclohexene, 1,3-cyclohexadiene and 1,4-cyclohexadiene.

The term “C₃-cycloalkenyl,” as used herein, means cycloprop-1-en-1-yl and cycloprop-2-en-1-yl.

The term “C₄-cycloalkenyl,” as used herein, means cyclobut-1-en-1-yl and cyclobut-2-en-1-yl.

The term “C₅-cycloalkenyl,” as used herein, means cyclopent-1-en-1-yl, cyclopent-2-en-1-yl, cyclopent-3-en-1-yl and cyclopenta-1,3-dien-1-yl.

The term “C₆-cycloalkenyl,” as used herein, means cyclohex-1-en-1-yl, cyclohex-2-en-1-yl, cyclohex-3-en-1-yl, cyclohexa-1,3-dien-1-yl, cyclohexa-1,4-dien-1-yl, cyclohexa-1,5-dien-1-yl, cyclohexa-2,4-dien-1-yl and cyclohexa-2,5-dien-1-yl.

The term “C₃-cycloalkyl,” as used herein, means cycloprop-1-yl.

The term “C₄-cycloalkyl,” as used herein, means cyclobut-1-yl.

The term “C₅-cycloalkyl,” as used herein, means cyclopent-1-yl.

The term “C₆-cycloalkyl,” as used herein, means cyclohex-1-yl.

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers of the compounds thereof.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds of this invention may also exist as a mixture of “Z” and “E” isomers.

Compounds of this invention may also exist as tautomers or equilibrium mixtures thereof wherein a proton of a compound shifts from one atom to another. Examples of tautomers include, but are not limited to, keto-enol, phenol-keto, oxime-nitroso, nitro-aci, imine-enamine and the like.

Compounds of this invention containing NH, C(O)OH, OH or SH moieties may have attached thereto prodrug-forming moieties. The prodrug-forming moieties are removed by metabolic processes and release the compounds having the freed NH, C(O)OH, OH or SH in vivo. Prodrugs are useful for adjusting such pharmacokinetic properties of the compounds as solubility and/or hydrophobicity, absorption in the gastrointestinal tract, bioavailability, tissue penetration, and rate of clearance.

Metabolites of compounds having formula (I) produced by in vitro or in vivo metabolic processes, may also have utility for treating diseases associated with overexpression or unregulation of protein kinases.

Certain precursor compounds which may be metabolized in vitro or in vivo to form compounds having formula (I) may also have utility for treating diseases associated with overexpression or unregulation of protein kinases.

Compounds having formula (I) may exist as acid addition salts, basic addition salts or zwitterions. Salts of compounds having formula (I) are prepared during their isolation or following their purification. Acid addition salts are those derived from the reaction of a compound having formula (I) with acid. Accordingly, salts including the acetate, adipate, alginate, bicarbonate, citrate, aspartate, benzoate, benzenesulfonate (besylate), bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate and undecanoate salts of the compounds having formula (I) are meant to be embraced by this invention. Basic addition salts of compounds are those derived from the reaction of the compounds having formula (I) with the bicarbonate, carbonate, hydroxide or phosphate of cations such as lithium, sodium, potassium, calcium and magnesium.

Compounds having formula (I) may be administered, for example, bucally, ophthalmically, orally, osmotically, parenterally (intramuscularly, intraperintoneally intrastemally, intravenously, subcutaneously), rectally, topically, transdermally, vaginally and intraarterially as well as by intraarticular injection, infusion, and placement in the body, such as, for example, the vasculature.

Therapeutically effective amounts of a compound having formula (I) depend on recipient of treatment, disease treated and severity thereof, composition comprising it, time of administration, route of administration, duration of treatment, potency, rate of clearance and whether or not another drug is co-administered. The amount of a compound having formula (I) used to make a composition to be administered daily to a patient in a single dose or in divided doses is from about 0.03 to about 200 mg/kg body weight. Single dose compositions contain these amounts or a combination of submultiples thereof.

Compounds having formula (I) may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants. perfumes, preservatives, propellants, releasing agents, sterilizing agents, sweeteners, solubilizers, wetting agents, mixtures thereof and the like.

Excipients for preparation of compositions comprising a compound having formula (I) to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

Compounds having formula (I) are also expected to be useful when used with alkylating agents, angiogenesis inhibitors, antibodies, antimetabolites, antimitotics, antiproliferatives, aurora kinase inhibitors, Bcr-Abl kinase inhibitors, biologic response modifiers, cyclin-dependent kinase inhibitors, cell cycle inhibitors, cyclooxygenase-2 inhibitors, leukemia viral oncogene homolog (ErbB2) receptor inhibitors, growth factor inhibitors, heat shock protein (HSP)-90 inhibitors, histone deacetylase (HDAC) inhibitors inhibitors, hormonal therapies, immunologicals, intercalating antibiotics, kinase inhibitors, mammalian target of rapomycin inhibitors, mitogen-activated extracellular signal-regulated kinase inhibitors, non-steroidal anti-inflammatory drugs (NSAID's), platinum chemotherapeutics, polo-like kinase inhibitors, proteasome inhibitors, purine analogs, pyrimidine analogs, receptor tyrosine kinase inhibitors, retinoids/deltoids plant alkaloids, topoisomerase inhibitors and the like.

Alkylating agents include altretamine, AMD-473, AP-5280, apaziquone, bendamustine, brostallicin, busulfan, carboquone, carmustine (BCNU), chlorambucil, Cloretazine™ (VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, treosulfan, trofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs vascular endothelial growth factor receptor tyrosine kinase (VEGFR) inhibitors and the like.

Aurora kinase inhibitors include AZD-1152, MLN-8054, VX-680 and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX™ (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFr immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), Herceptin® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024. 17-DMAG, geldanamycin, IPI-504, KOS-953, MYCOGRAB®, NCS-683664, PU24FCl, PU-3, radicicol, SNX-2112, STA-9090 VER49009 and the like.

MEK inhibitors include ARRY-142886, ARRY-438162 PD-325901, PD-98059 and the like.

mTOR inhibitors include AP-23573, CCI-779, everolimus, RAD-001, rapamycin, temsirolimus and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam) ibuprofin cream, ALEVE® and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin and the like.

Polo-like kinase inhibitors include BI-2536 and the like.

Thrombospondin analogs include ABT-510, ABT-567, ABT-898, TSP-1 and the like.

VEGFR inhibitors include AVASTIN® (bevacizumab), ABT-869, AEE-788, ANGIOZYME™, axitinib (AG-13736), AZD-2171, CP-547,632, IM-862, Macugen (pegaptamib), NEXAVAR® (sorafenib, BAY43-9006), pazopanib (GW-786034), (PTK-787, ZK-222584), SUTENT® (sunitinib, SU-11248), VEGF trap, vatalanib, ZACTIMA™ (vandetanib, ZD-6474) and the like.

Antimetabolites include ALIMTA® (premetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflomithine, EICAR, enocitabine, ethnylcytidine, fludarabine, hydroxyurea, 5-fluorouracil (5-FU) alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosate, pelitrexol, pentostatin, raltitrexed, Ribavirin, triapine, trimetrexate, S-1, tiazofurin, tegafur, TS-1, vidarabine, UFT and the like.

Antibiotics include intercalating antibiotics aclarubicin, actinomycin D, amrubicin, annamycin, adriamycin, BLENOXANE® (bleomycin), daunorubicin, CAELYX® or MYOCET® (doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab and and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL®, (flutamide), EVISTA® (raloxifene), fadrozole, FARESTON® (toremifene), FASLODEX® (fulvestrant), FEMARA®, (letrozole), formestane, glucocorticoids, HECTOROL® or RENAGEL® (doxercalciferol), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), predisone, PROPECIA® (finasteride), rilostane, SUPREFACT® (buserelin), TRELSTAR® (luteinizing hormone releasing hormone (LHRH)), vantas, VETORYL®, (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

Plant alkaloids include, but are not limited to, vincristine, vinblastine, vindesine, vinorelbine and the like.

Proteasome inhibitors include VELCADE® (bortezomib), mg132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b), or interferon gamma-n1, combinations thereof and the like. Other agents include ALFAFERONE®, BAM-002, BEROMUN® (tasonermin), BEXXAR® (tositumomab), CamPath® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010, melanoma vaccine, mitumomab, molgramostim, MYLOTARGTM (gemtuzumab ozognamicin), NEUPOGEN® (filgrastim), OncoVAC-CL, OvaRex® (oregovomab), pemtumomab (Y-muHMFG1), PROVENGE®, sargaramostim, sizofilan, teceleukin, TheraCys®, ubenimex, VIRULIZIN®, Z-F100, WF-10, PROLEUKIN®, (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth, or differentiation of tissue cells to direct them to have anti-tumor activity and include include krestin, lentinan, sizofiran, picibanil PF-3512676 (CpG-8954), ubenimex and the like.

Pyrimidine analogs include cytarabine (ara C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881, vinflunine, ZK-EPO and the like.

Compounds of the present invention are also intended to be used as a radiosensitizer that enhances the efficacy of radiotherapy. Examples of radiotherapy include, but are not limited to, external beam radiotherapy, teletherapy, brachtherapy and sealed and unsealed source radiotherapy.

Additionally, compounds having formula (I) may be combined with other chemptherapeutic agents such as ABRAXANE™ (ABI-007), ABT-100 (famesyl transferase inhibitor), ADVEXIN®, ALTOCOR® or MEVACOR® (lovastatin), AMPLIGEN® (poly I:poly C12U, a synthetic RNA), APTOSYN™ (exisulind), AREDIA® (pamidronic acid), arglabin, L-asparaginase, atamestane (1-methyl-3,17-dione-androsta-1,4-diene), AVAGE® (tazarotne), AVE-8062, BEC2 (mitumomab), cachectin or cachexin (tumor necrosis factor), canvaxin (vaccine), CeaVac™ (cancer vaccine), CELEUK® (celmoleukin), CEPLENE® (histamine dihydrochloride), CERVARIX™ (human papillomavirus vaccine), CHOP® (C: CYTOXAN® (cyclophosphamide); H: ADRIAMYCIN® (hydroxydoxorubicin); O: Vincristine (ONCOVIN®); P: prednisone), CyPat™, combrestatin A4P, DAB(389)EGF or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE®0 (T4N5 liposome lotion), discodermolide, DX-895 If (exatecan mesylate), enzastaurin, EPO906, GARDASIL® (quadrivalent human papillomavirus (Types 6, 11, 16, 18) recombinant vaccine), gastrimmune, genasense, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT®9(AE-941), NEUTREXIN® (trimetrexate glucuronate), NIPENT® (pentostatin), ONCONASE® (a ribonuclease enzyme), ONCOPHAGE® (melanoma vaccine treatment), OncoVAX (IL-2 Vaccine), ORATHECIN™ (rubitecan), OSIDEM® (antibody-based cell drug), OvaRex® MAb (murine monoclonal antibody), paditaxel, PANDIMEX™ (aglycone saponins from ginseng comprising 20(S)protopanaxadiol (aPPD) and 20(S)protopanaxatriol (aPPT)), panitumumab, PANVAC®-VF. (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), Taxoprexin® (DHA-paclitaxel), TELCYTA™ (TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFerade™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS™ (trabectedin), ZD-6126, ZINECARD® (dexrazoxane), zometa (zolendronic acid), zorubicin and the like.

To determine the inhibitory activity of representative compounds having formula (I) to protein kinases, the following assay was used:

In 384-well v-bottom polypropylene plates (Axygen #P-384-120SQ C), 10 μL recombinant Aurora Kinase-A (AurA, Upstate #14-511, 1 nM final concentration) was mixed with 10 μL biotinylated peptide substrate (Genemed, 2 μM final concentration), and various concentrations of representative compounds (2% DMSO final) in reaction buffer (25 mM HEPES, pH 7.5, 0.5 mM DTT, 10 mM mgCl₂ 100 μM Na₃VO₄, 0.075 mg/mL Triton X-100). The reaction was initiated by adding [³³P]-ATP (Perkin Elmer, 5 μM final concentration, 2 mCi/umol,). The reaction was quenched after 1 hour by addition of 50 μl stop buffer (50 mM EDTA, 2M NaCl final concentration). 80 μL of the stopped reactions were transferred to 384-well streptavidin-coated FlashPlates (Perkin Elmer, #SMP410A0001PK), incubated 10 minutes at ambient temperature, washed 3 times with 0.05% Tween-20/PBS using an ELX-405 automated plate washer (BioTek) and counted on a TopCount Scintillation Plate Reader (Packard).

IC₅₀ values values are shown in TABLE 1.

TABLE 1

| | | | | | |
|---|---|---|---|---|---|
| 0.0001 μM | 0.0004 μM | 0.0004 μM | 0.0006 μM | 0.0008 μM | 0.0009 μM |
| 0.0011 μM | 0.0013 μM | 0.0014 μM | 0.0016 μM | 0.0016 μM | 0.0021 μM |
| 0.0021 μM | 0.0021 μM | 0.0021 μM | 0.0023 μM | 0.0025 μM | 0.0026 μM |
| 0.0028 μM | 0.0033 μM | 0.0034 μM | 0.0034 μM | 0.0037 μM | 0.0041 μM |
| 0.0041 μM | 0.0044 μM | 0.0045 μM | 0.0046 μM | 0.0047 μM | 0.0050 μM |
| 0.0053 μM | 0.0056 μM | 0.0058 μM | 0.0064 μM | 0.0064 μM | 0.0065 μM |
| 0.0066 μM | 0.0069 μM | 0.0071 μM | 0.0072 μM | 0.0086 μM | 0.0089 μM |
| 0.0099 μM | 0.0110 μM | 0.0109 μM | 0.0110 μM | 0.0113 μM | 0.0117 μM |
| 0.0124 μM | 0.0124 μM | 0.0135 μM | 0.0146 μM | 0.0150 μM | 0.0154 μM |
| 0.0154 μM | 0.0167 μM | 0.0180 μM | 0.0187 μM | 0.0193 μM | 0.0203 μM |
| 0.0219 μM | 0.0255 μM | 0.0257 μM | 0.0266 μM | 0.0269 μM | 0.0275 μM |
| 0.0295 μM | 0.0300 μM | 0.0316 μM | 0.0318 μM | 0.0329 μM | 0.0333 μM |
| 0.0361 μM | 0.0381 μM | 0.0392 μM | 0.0438 μM | 0.0498 μM | 0.0610 μM |
| 0.0697 μM | 0.0723 μM | 0.1003 μM | 0.1036 μM | 0.1129 μM | 0.1201 μM |
| 0.1292 μM | 0.1324 μM | 0.1471 μM | 0.1579 μM | 0.1781 μM | 0.1788 μM |
| 0.2111 μM | 0.2671 μM | 0.2819 μM | 0.3422 μM | 0.3555 μM | 0.3621 μM |
| 0.3724 μM | 0.3766 μM | 0.5762 μM | 0.6109 μM | 0.6577 μM | 0.6941 μM |
| 0.7735 μM | 0.8917 μM | 0.9061 μM | 1.9591 μM | | |

These data demonstrate the utility of compounds having formula (I) as inhibitors of Aurora-kinase A.

To determine the activity of other representative compounds of the invention, Active Aurora A enzyme was incubated in wells of a 384 well plate with biotinylated STK substrate-2 (Upstate), 1 mM ATP, and various concentrations of inhibitors in a Hepes buffer, pH 7.4 containing MgCl₂, sodium othrovanadate, and Triton X-100. After 1 hour, the reaction was stopped with EDTA and anti-phospho-STK antibody Europium Cryptate (Upstate) and SA-XL665 (Upstate) were added to detect the phosphopeptide. The amount of phosphorylation was determined by the time-resolved fluorescence ratio of signals at 665 nm and 615 nm. The IC₅₀'s were calculated by an exponential fit of the inhibition values with the inhibitor concentrations using Assay Explorer software.

TABLE 2

| | | | | | |
|---|---|---|---|---|---|
| 0.0011 μM | 0.0024 μM | 0.0025 μM | 0.0055 μM | 0.0063 μM | 0.0187 μM |
| 0.0423 μM | 0.0543 μM | 0.0803 μM | 0.1076 μM | 0.1210 μM | 0.14665 μM |
| 0.1886 μM | 0.1995 μM | 0.2561 μM | 0.2593 μM | 0.2844 μM | 0.6520 μM |
| 0.8258 μM | 0.9535 μM | 7.6280 μM | | | |

It is expected that, because compounds having formula (I) inhibit the activity of Aurora-kinase A, they could also have utility as inhibitors of protein kinases having close structural homology to Aurora-kinase A such as, for example, Aurora-kinase B and Aurora-kinase C.

The structural homology between protein kinases A, B and C is reported in Nature Reviews/Cancer, Vol. 4 December, 2004.

Accordingly, compounds having formula (I) are expected to have utility in treatment of diseases during which protein kinases such as any or all Aurora-kinase family members are expressed.

Diseases involving overexpression or unregulation of Aurora-kinase family members include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hematological cancers (leukemias such as acute lymphocytic leukemia, chronic lymphocytic leukemia and chronic myeloid leukemia) and lymphomas), hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

It is also expected that compounds having formula (I) would inhibit the growth of cells derived from a cancer or neoplasm such as breast cancer (including estrogen-receptor positive breast cancer), colorectal cancer, endometrial cancer, lung cancer (including small cell lung cancer), lymphoma (including follicular or Diffuse Large B-cell), lymphoma (including non-Hodgkin's Iymphoma), neuroblastoma, ovarian cancer, prostate cancer (including hormone-insensitive prostate cancer) and testicular cancer (including germ cell testicular cancer).

It is also expected that compounds having formula (I) would inhibit the growth of cells derived from a pediatric cancer or neoplasm such as embryonal rhabdomyosarcoma, pediatric acute lymphoblastic leukemia, pediatric acute myelogenous leukemia, pediatric alveolar rhabdomyosarcoma, pediatric anaplastic ependymoma, pediatric anaplastic large cell lymphoma, pediatric anaplastic medulloblastoma, pediatric atypical teratoid/rhabdoid tumor of the central nervous syatem, pediatric biphenotypic acute leukemia, pediatric Burkitts lymphoma, pediatric cancers of Ewing's family of tumors such as primitive neuroectodermal rumors, pediatric diffuse anaplastic Wilm's tumor, pediatric favorable histology Wilm's tumor, pediatric glioblastoma, pediatric medulloblastoma, pediatric neuroblastoma, pediatric neuroblastoma-derived myelocytomatosis, pediatric pre-B-cell cancers (such as leukemia), pediatric psteosarcoma, pediatric rhabdoid kidney tumor, pediatric rhabdomyosarcoma, and pediatric T-cell cancers such as lymphoma and skin cancer.

For example, involvement of Aurora-kinases in bladder cancer, breast cancer, cervical cancer, colon cancer, endometrial cancer, esophageal cancer, lung cancer, ovarian cancer, pancreatic cancer, prostate cancer, rectal cancer, skin cancer, stomach cancer and thyroid cancer is reported in Nature Reviews/Cancer, Vol. 4 December, 2004.

Compounds having formula (I) may be made by synthetic chemical processes, examples of which are shown hereinbelow. It is meant to be understood that the order of the steps in the processes may be varied, that reagents, solvents and reaction conditions may be substituted for those specifically mentioned, and that vulnerable moieties may be protected and deprotected, as necessary.

Protecting groups for C(O)OH moieties include, but are not limited to, acetoxymethyl, allyl, benzoylmethyl, benzyl, benzyloxymethyl, tert-butyl, tert-butyldiphenylsilyl, diphenylmethyl, cyclobutyl, cyclohexyl, cyclopentyl, cyclopropyl, diphenylmethylsilyl, ethyl, para-methoxybenzyl, methoxymethyl, methoxyethoxymethyl, methyl, methylthiomethyl, naphthyl, para-nitrobenzyl, phenyl, n-propyl, 2,2,2-trichloroethyl, triethylsilyl, 2-(trimethylsilyl)ethyl, 2-(trimethylsilyl)ethoxymethyl, triphenylmethyl and the like.

Protecting groups for C(O) and C(O)H moieties include, but are not limited to, 1,3-dioxylketal, diethylketal, dimethylketal, 1,3-dithianylketal, O-methyloxime, O-phenyloxime and the like.

Protecting groups for NH moieties include, but are not limited to, acetyl, alanyl, benzoyl, benzyl(phenylmethyl), benzylidene, benzyloxycarbonyl (Cbz), tert-butoxycarbonyl (Boc), 3,4-dimethoxybenzyloxycarbonyl, diphenylmethyl, diphenylphosphoryl, formyl, niethanesulfonyl, para-methoxybenzyloxycarbonyl, phenylacetyl, phthaloyl, succinyl, trichloroethoxycarbonyl, triethylsilyl, trifluoroacetyl, trimethylsilyl, triphenylmethyl, triphenylsilyl, para-toluenesulfonyl and the like.

Protecting groups for OH and SH moieties include, but are not limited to, acetyl, allyl, allyloxycarbonyl, benzyloxycarbonyl (Cbz), benzoyl, benzyl, tert-butyl, tert-butyldimethylsilyl, tert-butyldiphenylsilyl, 3,4-dimethoxybenzyl, 3,4-dimethoxybenzyloxycarbonyl, 1,1-dimethyl-2-propenyl, diphenylmethyl, formyl, methanesulfonyl, methoxyacetyl, 4-methoxybenzyloxycarbonyl, para-methoxybenzyl, methoxycarbonyl, methyl, para-toluenesulfonyl, 2,2,2-trichloroethoxycarbonyl, 2,2,2-trichloroethyl, triethylsilyl, trifluoroacetyl, 2-(trimethylsilyl)ethoxycarbonyl, 2-trimethylsilylethyl, triphenylmethyl, 2-(triphenylphosphonio)ethoxycarbonyl and the like.

The following abbreviations have the meanings indicated. ADDP means 1,1′-(azodicarbonyl)dipiperidine; AD-mix-β means a mixture of (DHQD)₂PHAL, K₃Fe(CN)₆, K₂CO₃ and K₂SO₄); AIBN means 2,2′-azobis(2-methylpropionitrile); 9-BBN means 9-borabicyclo[3.3.1]nonane; Cp means cyclopentadiene; (DHQD)₂PHAL means hydroquinidine 1,4-phthalazinediyl diethyl ether; DBU means 1,8-diazabicyclo[5.4.0]undec-7-ene; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppa means diphenylphosphoryl azide; dppb means 1,4-bis(diphenylphosphino)butane; dppe means 1,2-bis(diphenylphosphino)ethane; dppf means 1,1′-bis(diphenylphosphino)ferrocene; dppm means 1,1-bis(diphenylphosphino)methane; EDAC means 1-(3-dimethylaminopropyl)-3-ethylcarbodiimide; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; LDA means lithium diisopropylamide; LHMDS means lithium bis(hexamethyldisilylamide); MP—BH₃ means macroporus triethylammonium methylpolystyrene cyanoborohydride; LAH means lithium aluminum hydride; NCS means N-chlorosuccinimide; PyBOP means benzotriazol-1-yloxytripyrrolidinophosphonium hexafluorophosphate; TDA-1 means tris(2-(2-methoxyethoxy)ethyl)amine; TEA means triethylamine; TFA means trifluoroacetic acid; THF means tetrahydrofuran; NCS means N-chlorosuccinimide; NMM means N-methylmorpholine; NMP means N-methylpyrrolidine; and PPh₃ means triphenylphosphine.

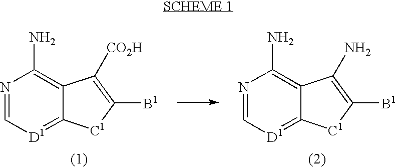

Compounds having formula (1) can be described herein and converted to compounds having formula (2) by the former and DPPA followed by hydrolysis of the product with water. The reactions are typically conducted in solvents such as benzene, toluene, THF, mixtures thereof and the like at temperatures between about 50° C. and 110° C.

Introduction of moieties represented by A¹ can be accomplished by reacting the compounds having formula (1), a compound having formula H₂NR¹ or HN(R¹)₂, a coupling agent and a base, with or without DMAP. Examples of coupling agents include DCC, EDCI and the like. Examples of bases include TEA, DIEA, pyridine and the like. The reactions are typically conducted in solvents such as THF, dichloromethane, DMF, DMSO, chloroform, mixtures thereof and the like at temperatures between about 0° C. and 25° C.

Introduction of moieties represented by A¹ can also be accomplished by reacting the compounds having formula (2) and the appropriate isocyanate, carbonyl chloride, sulfonyl chloride, carbamoyl chloride. The reactions are typically conducted in solvents such as THF, ethyl acetate, dichloromethane, DMF, DMSO, chloroform, mixtures thereof and the like at temperatures between about 0° C. and 110° C., depending on the reactivity of the starting materials.

The following examples are presented to provide what is believed to be the most useful and readily understood description of procedures and conceptual aspects of this invention.

### EXAMPLE 1A

A mixture of 5-amino-4-cyano-thiophene-3-carboxylic acid ethyl ester (2.4 g, prepared as described in Annali. di Chimica, 64, 833, 1974) in formamide (45 mL) at 170° C. was stirred for 8 hours and concentrated. The concentrate was flash chromatographed on silica gel with 10-50% ethyl acetate/hexanes.

### EXAMPLE 1B

A mixture of EXAMPLE 1A (0.62 g) and LiOH.H₂O (0.54 g) in THF (54 mL), water (13 mL) and methanol (13 mL) at 80° C. was stirred for 16 hours, cooled to ambient temperature and concentrated. The concentrate was taken up in water, cooled in an ice bath, stirred for 30 minutes and treated with 1N HCl until acidic, stirred for 30 minutes and filtered.

### EXAMPLE 1C

A mixture of 1-isocyanato-3-methylbenzene (0.6 mL) was added to a mixture of (4-aminophenyl)carbamic acid tert-butyl ester (1 g) in dichloromethane (48 mL) 0° C. The mixture was stirred for 30 minutes, warmed to ambient temperature, stirred for 24 hours and filtered. The filtrant was suspended in dichloromethane (80 mL), cooled in an ice bath, treated with TFA (5 mL), stirred for 15 minutes, warmed to ambient temperature, stirred for 18 hours and concentrated. The concentrate was concentrated twice from methanol and toluene.

### EXAMPLE 1D

Diisopropylethyl amine (0.3 mL) was added to a mixture of EXAMPLE 1B (0.2 g), EXAMPLE 1C (0.336 g) and HATU (0.452 g) in DMF (5.7 mL) at 0° C. The mixture was stirred for 0.5 hours, warmed to ambient temperature, stirred for for 20 hours, cooled to 0° C., diluted with water (80 mL), stirred for 1 hour and filtered. The filtrant was washed with water, dried and triturated with 2:1 dichloromethane/methanol. ¹H NMR (400 MHz, DMSO-d₆) δ 10.63 (s, 1H), 8.66 (s, 1H), 8.55 (s, 1H), 8.37 (s, 1H), 8.32 (s, 1H), 7.98 (br s, 2H), 7.63 (d, 2H), 7.46 (d, 2H), 7.30 (s, 1H), 7.18 (m, 2H), 6.79 (d, 1H), 2.28 (s, 3H).

### EXAMPLE 2

This example was prepared by substituting 1-fluoro-3-isocyanatobenzene for 1-isocyanato-3-methylbenzene in EXAMPLE 1C. ¹H NMR (500 MHz, DMSO-d₆) δ 10.67 (s, 1H), 8.89 (s, 1H), 8.76 (s, 1H), 8.37 (s, 1H), 8.32 (s, 1H), 8.02 (brs, 2H), 7.64 (d, 2H), 7.51 (s, 1H),7.47 (d, 2H), 7.30 (m, 1H), 7.12 (d, 1H), 6.78 (m, 1H).

### EXAMPLE 3

This example was prepared by substituting 1-fluoro-3-isocyanato-4-methylbenzene for 1-isocyanato-3-methylbenzene in EXAMPLE 1C. ¹H NMR (500 MHz, DMSO-d₆) δ 10.67 (s, 1H), 8.76 (s, 1H), 8.72 (s, 1H), 8.39 (s, 1H), 8.34 (s, 1H), 7.93 (s, 2H), 7.64 (d, 2H), 7.45 (m, 3H), 7.16 (m, 1H), 7.03 (d, 1H), 2.17 (s, 3H).

### EXAMPLE 4

This example was prepared by substituting 1-isocyanato-4-methylbenzene for 1-isocyanato-3-methylbenzene in EXAMPLE 1C. ¹H NMR (400 MHz, DMSO-d₆) δ 10.63 (d, 1H), 8.62 (d, 1H), 8.51 (d, 1H), 8.37 (d, 1H) 8.32 (d, 1H), 8.00 (brs, 2H), 7.62 (d, 2H), 7.46 (d, 2H), 7.33 (d, 2H), 7.08 (d, 2H), 2.24 (m, 3H).

### EXAMPLE 5

This example was prepared by substituting 1-fluoro-4-isocyanatobenzene for 1-isocyanato-3-methylbenzene in EXAMPLE 1C. ¹H NMR (500 MHz, DMSO-d₆) δ 10.66 (s, 1H), 8.68 (s, 2H), 8.37 (s, 1H), 8.32 (s, 1H), 7.92 (brs, 2H), 7.63 (d, 2H), 7.47 (d, 4H), 7.12 (t, 2H).

### EXAMPLE 6

This example was prepared by substituting 1-chloro-2-fluoro-4-isocyanatobenzene for 1-isocyanato-3-methylbenzene in EXAMPLE 1C. ¹H NMR (500 MHz, DMSO-d₆) δ 10.67 (s, 1H), 8.86 (s, 1H), 8.79 (s, 1H), 8.38 (s, 1H), 8.33 (s, 1H), 8.07 (brs, 2H), 7.81 (d, 1H), 7.64 (d, 2H), 7.47 (d, 2H), 7.32 (m, 2H).

### EXAMPLE 7

This example was prepared by substituting 1-ethyl-3-isocyanatobenzene for 1-isocyanato-3-methylbenzene in EXAMPLE 1C. ¹H NMR (300 MHz, DMSO-d₆) δ 10.64 (s, 1H), 8.65 (s, 1H), 8.57 (s, 1H), 8.38 (s, 1H), 8.33 (s, 1H), 7.63 (d, 2H), 7.46 (d, 2H), 7.24 (m, 5H), 6.82 (d, 1H), 2.58 (m, 2H), 1.18 (t, 3H).

### EXAMPLE 8

This example was prepared by substituting 1-chloro-3-isocyanatobenzene for 1-isocyanato-3-methylbenzene in EXAMPLE 1C. ¹H NMR (500 MHz, DMSO-d6) δ 10.67 (s, 1H), 8.87 (s, 1H), 8.78 (s, 1H), 8.38 (s. 1H). 8.32 (s, 1H), 7.72 (s, 1H), 7.64 (d, 2H), 7.68 (brs, 2H), 7.47 (d, 2H), 7.28 (m, 2H), 7.02 (d, 1H).

### EXAMPLE 9

This example was prepared by substituting 1-cyano-3-isocyanatobenzene for 1-isocyanato-3-methylbenzene in EXAMPLE 1C. ¹H NMR (300 MHz, DMSO-d6) δ 10.67 (s, 1H), 9.00 (s, 1H), 8.86 (s, 1H), 8.37 (s, 1H), 8.32 (s, 1H), 7.98 (m, 1H), 7.99 (brs, 2H), 7.66 (m, 3H), 7.46 (m, 4H).

### EXAMPLE 10

This example was prepared by substituting 1-fluoro-2-isocyanatobenzene for 1-isocyanato-3-methylbenzene in EXAMPLE 1C. ¹H NMR (500 MHz, DMSO-d₆) δ 10.68 (s, 1H), 9.11 (s, 1H), 8.54 (s, 1H), 8.38 (s, 1H), 8.32 (s, 1H), 8.16 (t, 1H), 7.83 (brs, 2H), 7.66 (d, ,2H), 7.48 (d, 2H), 7.24 (m, 1H), 7.14 (t, 1H), 7.01 (m, 1H).

### EXAMPLE 11

This example was prepared by substituting 1-isocyanato-3-trifluoromethylbenzene for 1-isocyanato-3-methylbenzene in EXAMPLE 1C. ¹H NMR (300 MHz, DMSO-d₆) δ 10.67 (s, 1H), 9.02 (s, 1H), 8.81 (s, 1H), 8.38 (s, 1H), 8.32 (s, 1H), 8.03 (s, 1H), 8.00 (brs, 2H), 7.65 (d, 2H), 7.56 (m, 2H), 7.48 (d, 2H), 7.31 (d, 1H).

### EXAMPLE 12

This example was prepared by substituting 1-fluoro-4-isocyanato-2-trifluoromethylbenzene for 1-isocyanato-3-methylbenzene in EXAMPLE 1C. ¹H NMR (300 MHz, DMSO-d₆) δ 10.67 (s, 1H), 9.01 (s, 1H), 8.83 (s, 1H), 8.38 (s, 1H), 8.32 (s, 1H), 8.02 (dd, 2H), 7.98 (brs, 2H), 7.65 (d, 2H), 7.46 (m, 3H).

### EXAMPLE 13

This example was prepared by substituting 1-isocyanato-3-methoxybenzene for 1-isocyanato-3-methylbenzene in EXAMPLE 1C. ¹H NMR (400 MHz, DMSO-d₆) δ 10.64 (s, 1H), 8.66 (s, 1H), 8.64 (s, 1H), 8.38 (s, 1H), 8.32 (s, 1H), 8.02 (brs, 2H), 7.63 (d, 2H), 7.47 (d, 2H), 7.19 (s, 1H), 7.16 (d, 1H), 6.93 (d, 1H), 6.55 (d, 1H), 3.74 (s, 3H).

### EXAMPLE 14

This example was prepared by substituting 1-fluoro-3-isocyanato-4-methylbenzene for 1-isocyanato-3-methylbenzene in EXAMPLE 1C. ¹H NMR (400 MHz, DMSO-d₆) δ 10.66 (s, 1H), 9.19 (s, 1H), 8.38 (s, 1H), 8.32 (s, 1H), 8.02 (s, 3H), 7.86 (dd, 1H), 7.66 (d, 2H), 7.49 (d, 2H), 7.19 (t, 1H), 6.74 (m, 1H), 2.23 (s, 3H).

### EXAMPLE 15

This example was prepared by substituting 1-isocyanato-4-trifluoromethylbenzene for 1-isocyanato-3-methylbenzene in EXAMPLE 1C. ¹H NMR (500 MHz, DMSO-d₆) δ 10.69 (s, 1H), 9.09 (s, 1H) 8.39 (s, 1H), 8.39 (s, 1H), 8.34 (s, 1H), 7.88 (brs, 2H), 7.65 (, 6H), 7.49 (d, 2H).

### EXAMPLE 16

This example was prepared by substituting 1-fluoro-2-isocyanato-4-methylbenzene for 1-isocyanato-3-methylbenzene in EXAMPLE 1C. ¹H NMR (500 MHz, DMSO-d₆) δ 10.67 (s, 1H), 9.09 (s, 1H), 8.47 (s, 1H), 8.39 (s, 1H), 8.33 (s, 1H), 8.00 (d, 1H), 7.81 (brs, 2H), 7.65 (d, 2H), 7.47 (d, 2H), 7.10 (m, 1H), 6.80 (s, 1H), 2.28 (s, 3H).

### EXAMPLE 17

This example was prepared by substituting 1-isocyanato-2-methylbenzene for 1-isocyanato-3-methylbenzene in EXAMPLE 1C. ¹H NMR (400 MHz, DMSO-d₆) δ 10.65 (s, 1H), 9.03 (s, 1H), 8.39 (s, 1H), 8.34 (s, 1H), 8.11 (brs, 2H), 7.89 (s, 1H), 7.84 (d, 1H), 7.64 (d, 2H), 7.48 (d, 2H), 7.15 (m, 2H), 6.94 (t, 1H), 2.25 (s, 3H).

### EXAMPLE 18

This example was prepared by substituting 1-isocyanato-4-methoxybenzene for 1-isocyanato-3-methylbenzene in EXAMPLE 1C. ¹H NMR (400 MHz, DMSO-d₆) δ 10.65 (s, 1H), 8.59 (s, 1H), 8.43 (s, 1H), 8.41 (s, 1H), 8.35 (s, 1H), 8.08 (brs, 2H), 7.62 (d, 2H), 7.46 (d, 2H), 7.35 (d, 2H), 6.87 (d, 2H), 3.72 (s, 3H).

### EXAMPLE 19

This example was prepared by substituting 1-isocyanato-3, 5-dimethylbenzene for 1-isocyanato-3-methylbenzene in EXAMPLE 1C. ¹H NMR (300 MHz, DMSO-d₆) δ 10.64 (s, 1H), 8.64 (s, 1H), 8.47 (s, 1H), 8.37 (s, 1H), 8.32 (s, 1H), 8.02 (brs, 2H), 7.63 (d, 2H), 7.46 (d, 2H), 7.07 (s, 2H), 6.61 (m, 1H), 2.23 (s, 6H).

### EXAMPLE 20

This example was prepared by substituting 1-fluoro-2-isocyanato-4-trifluoromethylbenzene for 1-isocyanato-3-methylbenzene in EXAMPLE 1C. ¹H NMR (500 MHz, DMSO-d₆) δ 10.69 (s, 1H), 9.21 (s, 1H), 8.88 (d, 1H), 8.64 (dd, 1H), 8.38 (s, 1G), 8.32 (s, 1H), 8.04 (brs, 2H), 7.68 (d, 2H), 7.50 (m, 3H), 7.39 (m, 1H).

### EXAMPLE 21

This example was prepared by substituting isocyanatobenzene for 1-isocyanato-3-methylbenzene in EXAMPLE 1C. ¹H NMR (400 MHz, DMSO-d₆) δ 10.64 (s, 1H), 8.67 (s, 1H), 8.63 (s, 1H), 8.38 (s, 1H), 8.33 (s, 1H), 8.01 (brs, 2H), 7.63 (d, 2H), 7.46 (m, 4H), 7.28 (m, 2H), 6.97 (m, 1H).

### EXAMPLE 22A

Bromine (0.75 mL) was added dropwise to mixture of 5-amino-4-cyanothiophene-3-carboxylic acid ethyl ester (2.9 g) in dichloromethane (150 mL) at 0° C. The mixture was stirred for 1.5 hours. diluted with dichloromethane, washed with 10% NaHSO₃ and brine and dried (MgSO₄), filtered and concentrated.

### EXAMPLE 22B

A mixture of EXAMPLE 22A (0.2 g), 1-methyl-4-(4,4,5,5-tetramethyl[1,3,2]dioxaborolan-2-yl)-1H-pyrazole (0.368 g), Na₂CO₃ (0.211 g), Pd(PPh₃)₄ (0.05 g) in DME (4 mL) and water (2 mL) at 120° C. was stirred in a sealed vial for 30 minutes in a Smith Synthesizer microwave oven (at 300 W), cooled to ambient temperature and partitioned between water and ethyl acetate. The extract was washed with brine, dried (MgSO₄), filtered and concentrated. The concentrate was flash chromatographed on silica gel with 1% methanot/dichloromethane.

### EXAMPLE 22C

This example was prepared by substituting EXAMPLE 22B for 5-amino-4-cyano-thiophene-3-carboxylic acid ethyl ester in EXAMPLE 1A and EXAMPLE 1B.

### EXAMPLE 22D

This example was prepared by substituting EXAMPLE 22C and 1-chloro-3-isocyanatobenzene for EXAMPLE 1B and 1-isocyanato-3-methylbenzene in EXAMPLES 1C and 1D, respectively. ¹H NMR (400 MHz, DMSO-d₆) δ 10.63 (s, 1H), 8.85 (s, 1H), 8.76 (s, 1H), 8.32 (s, 1H), 8.09 (s, 1H), 7.71 (m, 1H), 7.61 (s, 1H), 7.55 (d, 2H), 7.45 (d, 2H), 7.28 (m, 2H), 7.01 (m, 3H), 3.85 (s, 3H).

### EXAMPLE 23

This example was prepared by substituting EXAMPLE 22C for EXAMPLE 1B in EXAMPLE 1D. ¹H NMR (400 MHz, DMSO-d₆) δ 10.61 (s, 1H), 8.66 (s, 1H), 8.54 (s, 1H), 8.32 (s, 1H), 8.08 (s, 1H), 7.60 (s, 1H), 7.53 (d, 2H), 7.44 (d, 2H), 7.29 (s, 1H), 7.22 (d, 1H), 7.15 (t, 1H), 7.00 (brs, 2H), 6.78 (d, 1H), 3.84 (s, 3H), 2.27 (s, 3H).

### EXAMPLE 24

This example was prepared by substituting EXAMPLE 22C and 1-fluoro-2-isocyanato-4-methylbenzene for EXAMPLE 1B and 1-isocyanato-3-methylbenzene in EXAMPLES 1C and 1D, respectively. ¹H NMR (400 MHz, DMSO-d₆) δ 10.63 (s, 1H), 9.08 (s, 1H), 8.44 (d, 1H), 8.33 (s, 1H), 8.08 (s, 1H), 7.99 (dd, 1H), 7.60 (m, 1H), 7.55 (m, 2H), 7.45 (m, 2H), 7.09 (dd, 1H), 7.00 (brs, 2H), 6.80 (m, 1H), 3.85 (s, 3H), 2.27 (s, 3H).

### EXAMPLE 25

This example was prepared by substituting EXAMPLE 22C and 1-fluoro-4-isocyanato-2-trifluoromethylbenzene for EXAMPLE 1B and 1-isocyanato-3-methylbenzene in EXAMPLES 1C and 1D, respectively. ¹H NMR (400 MHz, DMSO-d₆) δ 10.64 (s, 1H), 9.01 (s, 1H), 8.82 (s, 1H), 8.32 (s, 1H), 8.09 (s, 1H), 8.00 (dd, 1H), 7.64 (m, 1H), 7.60 (s, 1H), 7.55 (d, 2H), 7.44 (m, 3H), 7.00 (brs, 2H), 3.85 (s, 3H).

### EXAMPLE 26

This example was prepared by substituting EXAMPLE 22C and 1-fluoro-2-isocyanato-4-trifluoromethylbenzene for EXAMPLE 1B and 1-isocyanato-3-methylbenzene in EXAMPLES 1C and 1D, respectively. ¹H NMR (500 MHz, DMSO-d₆) δ 10.67 (s, 1H), 9.21 (s, 1H), 8.87 (d, 1H), 8.63 (dd, 1H), 8.33 (s, 1H), 8.10 (s, 1H), 7.59 (m, 3H), 7.48 (m, 3H), 7.39 (m, 1H), 7.02 (brs, 2H), 3.85 (s, 3H).

### EXAMPLE 27

This example was prepared by substituting EXAMPLE 22C and 1-isocyanato-4-trifluoromethylbenzene for EXAMPLE 1B and 1-isocyanato-3-methylbenzene in EXAMPLES 1C and 1D, respectively. ¹H NMR (500 MHz, DMSO-d₆) δ 10.67 (s, 1H), 9.08 (s, 1H), 8.84 (s, 1H), 8.33 (s, 1H), 8.10 (s, 1H), 7.65 (m, 4H), 7.60 (s, 1H), 7.56 (d, 2H), 7.47 (d, 2H), 7.02 (brs, 2H), 3.85 (s, 3H).

### EXAMPLE 28

This example was prepared by substituting EXAMPLE 22C and 4-phenoxyphenylamine for EXAMPLES 1B and 1C, respectively, in EXAMPLE 1D. ¹H NMR (400 MHz, DMSO-d₆) δ 10.73 (s, 1H), 8.33 (s, 1H), 8.10 (s, 1H), 7.63 (t, 4H), 7.39 (t, 2H), 7.13 (t, 1H), 7.01 (m, 5H), 3.85 (s, 3H).

### EXAMPLE 29

This example was prepared by substituting 4-phenoxyphenylamine for EXAMPLE 1C in EXAMPLE 1D. ¹H NMR (300 MHz, DMSO-d₆) δ 10.76 (s, 1H), 8.39 (s, 1H), 8.32 (s, 1H), 7.74 (m, 2H), 7.39 (m, 2H), 7.13 (m, 1H), 7.07 (m, 2H), 7.01 (m, 2H).

### EXAMPLE 30A

A mixture of 1-fluoro-4-nitrobenzene (0.5 g), 3-methylphenol (0.383 g), 37% w/w KF—Al₂O₃ (0.4 g) and 18-crown-6 (0.093 g) in acetonitrile (6 mL) at reflux was stirred for 24 hours, cooled and partitioned between water and ethyl acetate. The extract was washed with water, dried (MgSO₄), filtered and concentrated. The concentrate was flash chromatographed on silica gel with 0-10% ethyl acetate/hexanes.

### EXAMPLE 30B

A mixture of EXAMPLE 30A (0.36 g), iron powder (0.45 g) and NH₄Cl (0.086 mg) in ethanol (46 mL), THF (17 mL) and water (6 mL) at 85° C. was stirred for 7 hours, cooled to ambient temperature, stirred for 18 hours, heated and filtered through diatomaceous earth (Celite®) while hot. The filtrate was concentrated and partitioned between water and ethyl acetate. The organic layer was washed with brine, dried (MgSO₄), filtered and concentrated. The concentrate was flash chromatographed on silica gel with 10% ethyl acetate/hexanes.

### EXAMPLE 30C

This example was prepared by substituting EXAMPLE 22C and 30B for EXAMPLES 1B and 1C, respectively, in EXAMPLE 1D. ¹H NMR (400 MHz, DMSO-d₆) δ 10.73 (s, 1H), 8.33 (s, 1H), 8.10 (s, 1H), 7.63 (m, 3H), 7.26 (t, 1H), 7.03 (m, 2H), 6.99 (brs, 2H), 6.95 (d, 1H), 6.84 (m, 1H), 6.80 (dd, 1H), 3.85 (s, 3H), 2.29 (s, 3H).

### EXAMPLE 31

This example was prepared by substituting EXAMPLE 22C and 4-(4-chlorophenoxy)-phenylamine (prepared by substituting 4-chlorophenol for 3-methylphenol in EXAMPLE 30B) for EXAMPLES 1B and 1C, respectively, in EXAMPLE 1D. ¹H NMR (400 MHz, DMSO-d₆) δ 10.76 (s, 1H), 8.33 (s, 1H), 8.10 (s, 1H), 7.66 (d, 2H), 7.61 (s, 1H), 7.42 (d, 2H), 7.08 (d, 2H), 7.03 (d, 2H), 6.98 (brs, 2H), 3.85 (s, 3H).

### EXAMPLE 32

This example was prepared by substituting EXAMPLE 22C and 4-(4-methyl-phenoxy)phenylamine (prepared by substituting 4-methylphenol for 3-methylphenol in EXAMPLE 30B) for EXAMPLES 1B and 1C, respectively, in EXAMPLE 1D. ¹H NMR (400 MHz, DMSO-d₆) δ 10.71 (s, 1H), 8.33 (s, 1H), 8.10 (s, 1H), 7.63 (s, 1H), 7.60 (brs, 2H), 7.19 (d, 2H), 6.96 (m, 6H), 3.85 (s, 3H), 2.29 (s, 3H).

### EXAMPLE 33

This example was prepared by substituting EXAMPLE 22C and 4-(3-chlorophenoxy)-phenylamine (prepared by substituting 3-chlorophenol for 3-methylphenol in EXAMPLE 30B) for EXAMPLES 1B and 1C, respectively, in EXAMPLE 1D. ¹H NMR (400 MHz, DMSO-d₆) δ 10.78 (s, 1H), 8.33 (s, 1H), 8.10 (s, 1H), 7.68 (d, 2H), 7.62 (s, 1H), 7.40 (t, 1H), 7.19 (m, 1H), 7.11 (d, 2H), 7.06 (t, 1H), 6.97 (m, 3H), 3.85 (s, 3H).

### EXAMPLE 34

This example was prepared by substituting EXAMPLE 22C and 4-phenylsulfanylphenylamine for EXAMPLES 1B and 1C, respectively, in EXAMPLE 1D. ¹H NMR (400 MHz, DMSO-d₆) δ 10.84 (s, 1H), 8.33 (s, 1H), 8.09 (s, 1H), 7.67 (m, 2H), 7.59 (d, 1H), 7.38 (m, 4H), 7.28 (m, 3H), 6.96 (brs, 2H), 3.84 (s, 3H).

### EXAMPLE 35

This example was prepared by substituting 4-(4-methylphenoxy)phenylamine (prepared by substituting 4-methylphenol for 3-methylphenol in EXAMPLE 30B) for EXAMPLE 1C in EXAMPLE 1D. ¹H NMR (300 MHz, DMSO-d₆) δ 10.73 (s, 1H), 8.38 (s, 1H), 8.31 (s, 1H), 7.96 (m, 2H), 7.71 (d, 2H), 7.19 (d, 2H), 7.01 (d, 2H), 6.91 (d, 2H), 2.29 (s, 3H).

### EXAMPLE 36

This example was prepared as described in EXAMPLES 22B and 22C by substituting 4-(4,4,5,5-tetramethyl[1,3,2]dioxaborolan-2-yl)-1H-pyrazole for 1-methyl-4-(4,4,5,5-tetramethyl[1,3,2]dioxaborolan-2-yl)-1H-pyrazole in EXAMPLE 22B and coupling the product therefrom as described in EXAMPLE 1D by substituting 4-phenoxyphenylaamine for EXAMPLE 1C. ¹H NMR (400 MHz, DMSO-d₆) δ 10.74 (s, 1H), 8.33 (s, 1H), 8.09 (s, 1H), 7.70 (s, 1H), 7.65 (m, 2H), 7.38 (m, 2H), 7.13 (t, 1H), 7.03 (m, 7H).

### EXAMPLE 37

This example was prepared as described in EXAMPLES 22B and 22C by substituting 4,4,5,5-tetramethyl-2-thiophen-3-yl[1,3,2]dioxaborolane for 1-methyl-4-(4,4,5,5-tetramethyl[1.3.2]dioxaborolan-2-yl)-1H-pyrazole in EXAMPLE 22B and coupling the product therefrom as described in EXAMPLE 1D by substituting 4-phenoxyphenylamine for EXAMPLE 1C. ¹H NMR (500 MHz, DMSO-d₆) δ 10.71 (s, 1H), 8.37 (s, 1H), 7.85 (m, 1H), 7.68 (m, 1H), 7.58 (d, 2H), 7.39 (t, 2H), 7.28 (d, 1H), 7.13 (t, 2H), 7.01 (t, 5H).

### EXAMPLE 38A

A mixture of EXAMPLE 22A (0.1 g) and 1-methylpiperazine (1 mL) was stirred at 130° C. for 8 hours, cooled and partitioned between water and ethyl acetate. The extract was washed with water and brine and dried (MgSO₄), filtered and concentrated.

### EXAMPLE 38B

This example was prepared by substituting EXAMPLE 38A for 5-amino-4-cyano-thiophene-3-carboxylic acid ethyl ester in EXAMPLES 1A-D. ¹H NMR (400 MHz, DMSO-d₆) δ 10.60 (s, 1H), 8.65 (s, 1H), 8.56 (s, 1H), 8.20 (s, 1H), 7.64 (d, 2H), 7.50 (s, 2H), 7.46 (d, 2H), 7.30 (s, 1H), 7.23 (m, 1H), 7.15 (t, 1H), 6.79 (d, 1H), 3.12 (m, 4H), 2.43 (m, 4H), 2.28 (s, 3H), 2.18 (s, 3H).

### EXAMPLE 39A

1M borane THF in THF (0.28 mL) was added to mixture of N-(4-(formylamino)phenyl)-N′-(3-methylphenyl)urea (0.05 g, prepared by substituting formic acid for EXAMPLE 1B in EXAMPLE 1D) in THF (2 mL) at 0° C. The mixture was stirred for 1.5 hours at ambient temperature, cooled to 0° C., treated with methanolic HCl (2 mL), stirred at reflux for 1 hour, cooled to ambient temperature and concentrated. The concentrate was reconcentrated twice from methanol then flash chromatographed on silica 5% methanol/dichloromethane.

### EXAMPLE 39B

This example was prepared by substituting EXAMPLE 39A for EXAMPLE 1C in EXAMPLE 1D. ¹H NMR (500 MHz, DMSO-d₆) δ 8.70 (s, 1H), 8.57 (s, 1H), 8.31 (s, 1H), 7.37 (d, 4H), 7.35 (brs, 2H), 7.26 (s, 2H), 7.20 (d, 1H), 7.14 (m, 1H), 7.09 (d, 3H), 6.78 (d, 1H), 3.42 (s, 3H).

### EXAMPLE 40A

A mixture of 3-bromothieno[3,2-c]pyridin-4-ylamine (2 g, prepared as described in WO 05/010009) and PdCl₂(dppf)-dichloromethane (0.715 g) in methanol (60 mL) and triethylamine (3.7 mL) in a sealed tube under CO (60 psi) was stirred at 100° C. for 16 hours, cooled to ambient temperature, filtered and concentrated. The concentrate was triturated with water and filtered.

### EXAMPLE 40B

A suspension of EXAMPLE 40A (0.87 g) in 9M HCl (50 mL) was heated to reflux for 18 hours, filtered hot and concentrated.

### EXAMPLE 40C

This example was prepared by substituting EXAMPLE 40B and 4-phenoxyphenylamine for EXAMPLES 1B and 1C, respectively, in EXAMPLE 1D. ¹H NMR (DMSO-d₆, 300 MHz) δ 10.79 (s, 1H), 8.28 (s, 1H), 7.87 (d, 1H), 7.77 (d, 2H), 7.39 (m, 2H), 7.25 (d, 1H), 7.13 (t, 1H), 7.07 (d, 2H), 7.01 (d, 2H), 6.80 (brs, 2H).

### EXAMPLE 41

This example was prepared by substituting EXAMPLE 40B and 3-phenoxyphenylamine for EXAMPLES 1B and 1C, respectively, in EXAMPLE 1D. ¹H NMR (DMSO-d₆, 300 MHz) δ 10.82 (s, 1H), 8.28 (s, 1H), 7.86 (d, 1H), 7.56 (d, 1H), 7.40 (m, 4H), 7.24 (d, 1H), 7.17 (t, 1H), 7.07 (d, 2H), 6.81 (d, 1H), 6.73 (brs, 2H).

### EXAMPLE 42

This example was prepared by substituting EXAMPLE 40B and 4-benzylphenylamine for EXAMPLES 1B and 1C, respectively, in EXAMPLE 1D. ¹H NMR (DMSO-d₆, 300 MHz) δ 10.69 (s, 1H), 8.25 (s, 1H), 7.86 (d, 1H), 7.66 (d, 2H), 7.24 (m, 8H), 6.78 (brs, 2H). 3.93 (s, 2H).

### EXAMPLE 43

This example was prepared by substituting EXAMPLE 40B for EXAMPLE 1B in EXAMPLE 1D. ¹H NMR (DMSO-d₆, 300 MHz) δ 10.67 (s, 1H), 8.67 (s, 1H), 8.57 (s, 1H), 8.26 (s, 1H), 7.87 (d, 1H), 7.67 (d, 2H), 7.47 (d, 2H), 7.30 (s, 1H), 7.23 (m, 2H), 7.15 (t, 1H), 6.83 (s, 2H), 6.80 (d, 1H), 2.28 (s, 3H).

### EXAMPLE 44

This example was prepared by substituting EXAMPLE 40B and N-(4-aminophenyl)benzamide for EXAMPLES 1B and 1C, respectively in EXAMPLE 1D. ¹H NMR (DMSO-d₆, 300 MHz) δ 10.76 (s, 1H), 10.28 (s, 1H), 8.29 (s, 1H), 7.98 (d, 2H), 7.88 (d, 1H), 7.81 (d, 2H), 7.74 (d, 2H), 7.56 (m, 3H), 7.26 (d, 1H), 6.83 (brs, 2H).

### EXAMPLE 45A

A mixture of EXAMPLE 40B (0.73 g) in DMF (30 mL) at ambient temperature was treated with NIS (1.42 g), stirred for 18 hours, diluted with water, treated with 10% aqueous Na₂S₂O₃ and filtered.

### EXAMPLE 45B

This example was prepared by substituting EXAMPLE 45A and (4-aminophenyl)carbamic acid tert-butyl ester for EXAMPLES 1B and 1C, respectively, in EXAMPLE 1D. The Boc protecting group was removed by treatment of the product therefrom with TFA as described in EXAMPLE 1C.

### EXAMPLE 45C

A mixture of EXAMPLE 45B (1.93 g), 1-methyl-4-(4,4,5,5-tetramethyl[1,3,2]dioxaborolan-2-yl)-1H-pyrazole (1.08 g), PdCl₂(dppf) (0.19 g) and Na₂CO₃ (1.3 g) in DME (30 mL) and water (10 mL) at 80° C. was stirred for 18 hours, cooled, treated with water and ethyl acetate and filtered.

### EXAMPLE 45D

A mixture of EXAMPLE 45C (0.03 g) in DMF (0.5 mL) at −20° C. was treated with 1-isocyanato-3-methylbenzene (0.1 mL), the warmed to ambient temperature and stirred for 18 hours and filtered. The filtrant was flash chromatographed on silica gel with 0-8% methanol/dichloromethane. ¹H NMR (300 MHz, DMSO-d₆) δ 10.70 (s, 1H), 8.67 (s, 1H), 8.57 (s, 1H), 8.33 (s, 1H), 8.14 (d, 1H), 8.06 (s, 1H), 7.86 (d, 1H), 7.67 (d, 2H), 7.47 (d, 2H), 7.30 (s, 1H), 7.23 (d, 1H), 7.15 (t, 1H), 6.82 (s, 2H), 6.79 (d, 1H), 3.93 (s, 3H), 2.28 (s, 3H).

### EXAMPLE 46

This example was prepared by substituting EXAMPLE 45B and N-(4-aminophenyl)benzamide for EXAMPLES 1B and 1C, respectively, in EXAMPLE 1D then by substituting the product thereform for EXAMPLE 45B in EXAMPLE 45C. ¹H NMR (DMSO-d₆, 300 MHz) δ 10.80 (s, 1H), 10.29 (s, 1H), 8.36 (s, 1H), 8.14 (s, 1H), 8.06 (s, 1H), 7.97 (d, 2H), 7.87 (s, 1H), 7.77 (m, 4H), 7.56 (m, 3H), 6.83 (brs, 2H), 3.93 (s, 3H).

### EXAMPLE 47

This example was prepared by substituting EXAMPLE 45B and 4-phenoxyphenylamine for EXAMPLES 1B and 1C, respectively, in EXAMPLE 1D then by substituting the product thereform for EXAMPLE 45B in EXAMPLE 45C. ¹H NMR (DMSO-d₆, 300 MHz) δ 10.84 (s, 1H), 8.36 (s, 1H), 8.14 (s, 1H), 8.06 (s, 1H), 7.87 (s, 1H), 7.80 (d, 2H), 7.39 (m, 2H), 7.13 (t, 1H), 7.09 (d, 2H), 7.03 (d, 2H), 6.81 (brs, 2H), 3.93 (s, 3H).

### EXAMPLE 48

This example was prepared by substituting EXAMPLE 45B and 3-phenoxyphenylamine for EXAMPLES 1B and 1C, respectively, in EXAMPLE 1D then by substituting the product therefrom for EXAMPLE 45B in EXAMPLE 45C. ¹H NMR (DMSO-d₆, 300 MHz) δ 10.85 (s, 1H), 8.35 (s, 1H), 8.13 (s, 1H), 8.05 (s, 1H), 7.85 (s, 1H), 7.58 (d, 1H), 7.46 (m, 2H), 7.42 (d, 2H), 7.17 (t, 1H), 7.08 (d, 2H), 6.81 (m, 1H), 6.73 (brs, 2H), 3.92 (s, 3H).

### EXAMPLE 49

This example was prepared by substituting EXAMPLE 45B and 4-benzylphenylamine for EXAMPLES 1B and 1C, respectively, in EXAMPLE 1D then by substituting the product therefrom for EXAMPLE 45B in EXAMPLE 45C. ¹H NMR (DMSO-d₆, 300 MHz) δ 10.75 (s, 1H), 8.32 (s, 1H), 8.13 (s, 1H), 8.05 (s, 1H), 7.86 (s, 1H), 7.67 (d, 2H), 7.32-7.18 (m, 7H), 6.79 (brs, 2H), 3.94 (s, 2H), 3.93 (s, 3H).

### EXAMPLE 50A

This example was prepared as described in EXAMPLE 40A and EXAMPLE 40B, except substituting 5-iodo-7-(4-(4-methyl-piperazin-1-yl)cyclohexyl)-7H-pyrrolo[2,3-d]pyrimidin-4-ylamine (WO2005/74603) for 3-bromo-thieno[3,2-c]pyridin-4-ylamine in EXAMPLE 40A.

### EXAMPLE 50B

This example was prepared by substituting EXAMPLE 50A and 4-phenoxyphenylamine for EXAMPLES 1B and 1C, respectively, in EXAMPLE 1D. ¹HNMR (DMSO-d₆, 300 MHz) δ 10.13 (s, 1H), 8.30 (s, 1H), 8.12 (s, 1H), 7.93 (brs, 2H), 7.70-7.74 (m, 1H), 7.66-7.70 (m, 1H), 7.36-7.44 (m, 2H), 7.08-7.17 (m, 1H), 6.98-7.07 (m, 4H), 4.60-4.72 (m, 1H), 3.32 (hidden, 1H), 2.48-2.59 (m, 4H), 2.33-2.48 (m, 4H), 2.15-2.23 (m, 4H), 1.96-2.15 (m, 3H), 1.74-1.85 (m, 2H), 1.53-1.68 (m, 2H).

### EXAMPLE 51

This example was prepared by substituting EXAMPLE 50A and 3-phenoxyphenylamine for EXAMPLES 1B and 1C, respectively, in EXAMPLE 1D. ¹HNMR (DMSO-d₆, 300 MHz) δ 10.12 (s, 1H), 8.28 (s, 1H), 8.11 (s, 1H), 7.87 (bs, 2H), 7.49-7.56 (m, 1H), 7.33-7.46 (m, 4H), 7.13-7.21 (m, 1H), 7.06-7.10 (m, 1H), 7.03-7.06 (m, 1H), 6.74-6.81 (m, 1H), 4.58-4.71 (m, 1H), 2.32-2.60 (m, 8H), 2.25-2.30 (m, 1H), 1.93-2.24 (m, 7H), 1.73-1.85 (m, 2H), 1.52-1.68 (m, 2H).

### EXAMPLE 52

This example was prepared by substituting EXAMPLE 50A for EXAMPLE 1B in EXAMPLE 1D. ¹H NMR (DMSO-d₆, 300 MHz) δ 10.03 (s, 1H), 8.64 (s, 46H), 8.56 (s, 1H), 8.30 (s, 1H), 8.11 (s, 1H), 7.99 (bs, 2H), 7.59-7.63 (m, 1H), 7.56-7.59 (m, 1H), 7.45-7.48 (m, 1H), 7.42-7.45 (m, 1H), 7.31 (s, 1H), 7.20-7.26 (m, 1H), 7.15 (t, 1H), 6.79 (d, 1H), 4.59-4.72 (m, 1H), 2.37-2.51 (m, 9H), 2.28 (s, 3H), 2.20 (s, 3H), 1.96-2.16 (m, 4H), 1.74-1.86 (m, 2H), 1.53-1.68 (m, 2H).

### EXAMPLE 53

This example was prepared by substituting EXAMPLE 50A and 4-(4-aminophenylsulfanyl)phenylamine for EXAMPLES 1B and 1C, respectively, in EXAMPLE 1D. ¹HNMR(DMSO-d₆, 300 MHz) δ 10.09 (s, 1H), 8.29 (s, 1H), 8.11 (s, 1H), 7.88 (bs, 2H), 7.59-7.63 (m, 1H), 7.56-7.59 (m, 1H), 7.17-7.21 (m, 1H), 7.14-7.17 (m, 1H), 7.08-7.11 (m, 1H), 7.05-7.08 (m, 1H), 6.81-6.83 (m, 1H), 6.79-6.81 (m, 1H), 5.47 (s, 2H), 4.58-4.71 (m, 1H), 2.56-2.70 (m, 9H), 2.20 (s, 3H), 1.94-2.16 (m, 4H), 1.73-1.85 (m, 2H), 1.52-1.67 (m, 2H).

### EXAMPLE 54

This example was prepared by substituting EXAMPLE 50A and 4-benzylphenylamine for EXAMPLES 1B and 1C, respectively, in EXAMPLE 1D. ¹HNMR (DMSO-d₆, 300 MHz) δ 10.04 (s, 1H), 8.29 (s, 1H), 8.11 (s, 1H), 7.92 (bs, 2H), 7.60-7.63 (m, 1H), 7.57-7.60 (m, 1H), 7.15-7.34 (m, 7H), 4.59-4.72 (m, 1H), 3.93 (s, 2H), 2.37-2.59 (m, 8H), 2.25-2.29 (m, 1H), 2.22 (s, 3H), 1.94-2.15 (m, 4H), 1.74-1.85 (m, 2H), 1.53-1.68 (m, 2H).

### EXAMPLE 55

This example was prepared by substituting EXAMPLE 50A and 4-benzenesulfonylphenylamine for EXAMPLES 1B and 1C, respectively, in EXAMPLE 1D. ¹HNMR(DMSO-d₆, 300 MHz) δ 10.41 (s, 1H), 8.34 (s, 1H), 8.13 (s, 1H), 7.96 (s, 5H), 7.92-7.94 (m, 1H), 7.87 (bs, 2H), 7.59-7.73 (m, 3H), 4.59-4.72 (m, 1H), 2.36-2.48 (m, 8H), 2.16-2.22 (m, 4H), 1.94-2.16 (m, 4H), 1.75-1.85 (m, 2H), 1.53-1.69 (m, 2H).

### EXAMPLE 56A

This example was prepared as described in EXAMPLES 40A and 40B by substituting 3-iodo-1-(4-morpholin-4-ylcyclohexyl)-1 H-pyrazolo[3,4-d]pyrimidin-4-ylamine (prepared as described in WO 05/74603) for 3-bromothieno[3,2-c]pyridin-4-ylamine in EXAMPLE 40A.

### EXAMPLE 56B

This example was prepared by substituting EXAMPLE 56A and 4-phenoxyphenylamine for EXAMPLES 1B and 1C, respectively, in EXAMPLE 1D. ¹H NMR (DMSO-d₆, 300 MHz) δ 10.41 (s, 1H), 8.53 (s, 1H), 8.25 (s, 1H), 8.06 (bs, 1H), 7.83-7.87 (m, 1H), 7.79-7.83 (m, 1H), 7.36-7.45 (m, 2H), 7.10-7.17 (m, 1H), 6.99-7.09 (m, 4H), 4.83-4.96 (m, 1H), 3.55-3.63 (m, 4H), 2.47-2.57 (m, 4H), 2.29-2.42 (m, 1H), 2.09-2.29 (m, 2H), 1.96-2.09 (m, 4H), 1.40-1.57 (m, 2H).

### EXAMPLE 57A

This example was prepared by substituting 3-bromo-7-iodo-thieno[3,2-c]pyridin-4-ylamine (prepared as described in WO 05/10009) for EXAMPLE 45B in EXAMPLE 45C.

### EXAMPLE 57B

A mixture of 4-ethynylphenylamine (0.3 g), 4,4,5,5-tetramethyl[1,3,2]-dioxaborolane (0.56 mL) and ZrCp₂ClH (0.083 g) in THF (6 mL) was stirred at 50° C. for 1.5 hours and concentrated. The concentrate was flash chromatographed on silica gel with 30% ethyl acetate/hexanes.

### EXAMPLE 57C

This example was prepared by substituting EXAMPLES 57A and 57B for EXAMPLE 45B and 1-methyl-4-(4,4,5,5-tetramethyl[1,3,2]dioxaborolan-2-yl)-1H-pyrazole, respectively, in EXAMPLE 45C. ¹H NMR (300 MHz, DMSO-d₆) δ 3.92 (s, 3H) 5.32 (s, 2H) 6.01 (s, 2H) 6.57 (d, 2H) 6.86 (d, 1H) 7.27-7.42 (m, 3H) 7.60 (s, 1H) 7.85 (s, 1H) 8.11 (s, 1H).

### EXAMPLE 58A

This example was prepared by substituting EXAMPLE 57B for EXAMPLE 45C in EXAMPLE45D.

### EXAMPLE 58B

This example was prepared by substituting EXAMPLES 57A and 58A for EXAMPLE 45B and 1-methyl-4-(4,4,5 5-tetramethyl[1,3,2]dioxaborolan-2-yl)-1H-pyrazole, respectively, in EXAMPLE 45C. ¹H NMR (300 MHz, DMSO-d₆) δ 2.28 (s, 3H) 3.93 (s, 3H) 6.08 (s, 2H) 6.80 (d, 1H) 7.00 (d, 1H) 7.16 (t, 1H) 7.20-7.27 (m, 1H) 7.31 (s, 1H) 7.44-7.54 (m, 2H) 7.54-7.68 (m, 3H) 7.73 (s, 1H) 7.86 (s, 1H) 8.01 (s, 1H) 8.12 (s, 1H) 8.62 (s, 1H) 8.79 (s, 1H).

### EXAMPLE 59

This example was prepared by substituting 1-ethynyl-4-phenoxybenzene for 4-ethynylphenylamine in EXAMPLE 57B then substituting the product therefrom and EXAMPLE 57A for 1-methyl-4-(4,4,5,5-tetramethyl [1,3,2]dioxaborolan-2-yl)-1H-pyrazole and EXAMPLE 45B, respectively, in EXAMPLE 45C. ¹H NMR (300 MHz, DMSO-d₆) δ 3.93 (s, 3H) 6.10 (s, 2H) 6.99-7.11 (m, 5H) 7.16 (t, 1H) 7.36-7.47 (m, 2H) 7.61-7.78 (m, 4H) 7.86 (s, 1H) 8.02 (s, 1H) 8.12 (s, 1H).

### EXAMPLE 60

This example was prepared by substituting EXAMPLE 57A and 2-(2-biphenyl-4-yl-vinyl)-4,4,5,5-tetramethyl[1,3,2]dioxaborolane for EXAMPLE 45B and 1-methyl-4-(4,4,5,5-tetramethyl[1,3,2]dioxaborolan-2-yl)-1H-pyrazole, respectively, in EXAMPLE 45C. ¹H NMR (300 MHz, DMSO-d₆) δ 3.93 (s, 3 H) 6.11 (s, 2 H) 7.13 (d, 1H) 7.33-7.42 (m, 1H) 7.48 (t, 2H) 7.68-7.85 (m, 8H) 7.87 (s, 1H) 8.03 (s, 1H) 8.13 (s, 1H).

EXAMPLES 61-65 were prepared following the procedures of EXAMPLE 45D and substituting the appropriate isocyanate (X) for 1-isocyanato-3-methylbenzene.

### EXAMPLE 61

X=1-isocyanatobenzene. ¹H NMR (300 MHz, DMSO-d₆) δ 10.72 (s, 1H), 8.70 (s, 1H), 8.66 (s, 1H), 8.33 (s, 1H), 8.14 (s, 1H), 8.06 (s, 1H), 7.87 (s, 1H), 7.67 (d, J=8.8 Hz, 2H), 7.43-7.50 (m, 4H), 7.25-7.31 (m, 2H), 6.97 (t, J=7.3Hz, 1H), 6.83 (s, 2H), 3.93 (s, 3H).

### EXAMPLE 62

X=1-fluoro-3-isocyanatobenzene. ¹H NMR (300 MHz, DMSO-d₆) δ 10.73 (s, 1H), 8.88 (s, 1H), 8.75 (s, 1H), 8.34 (s, 1H), 8.14 (s, 1H), 8.06 (s, 1H), 7.87 (s, 1H), 7.68 (d, J=8.8 Hz, 2H), 7.44-7.53 (m, 3H), 7.31 (td, J=8.1, 6.8 Hz, 1H), 7.12 (ddd, J=8.1, 2.0, 0.7 Hz, 1H), 6.83 (s, 2H), 6.74-6.82 (m, 1H), 3.93 (s, 3H).

### EXAMPLE 63

X=isocyanatocyclohexane. ¹H NMR (300 MHz, DMSO-d₆) δ 10.65 (s, 1H), 8.35 (s, 1H), 8.31 (s, 1H), 8.13 (s, 1H), 8.05 (s, 1H), 7.86 (s, 1H), 7.59 (d, J=9.2Hz, 2H), 7.38 (d, J=9.2 Hz, 2H), 6.82 (s, 2H), 6.08 (d, J=8.1 Hz, 1H), 3.93 (s, 3H), 1.75-1.85 (m, 2H), 1.60-1.72 (m, 2H), 1.47-1.59 (m, 1H), 1.08-1.40 (m, 5H).

### EXAMPLE 64

X=1-isocyanato-4-methylbenzene. ¹H NMR (300 MHz, DMSO-d₆) δ 10.71 (s, 1H), 8.67 (s, 1H), 8.57 (s, 1H), 8.33 (s, 1H), 8.14 (d, J=0.7Hz, 1H), 8.06 (s, 1H), 7.87 (d, J=1.0 Hz, 1H), 7.66 (d, J=9.2 Hz, 2H), 7.46 (d, J=9.2 Hz, 2H), 7.34 (d, J=8.5 Hz, 2H), 7.08 (d, J=8.5 Hz, 2H), 6.83 (s, 2H), 3.93 (s, 3H), 2.24 (s, 3H).

### EXAMPLE 65

X=1-isocyanato-2-methylbenzene. ¹H NMR (300 MHz, DMSO-d₆) δ 10.72 (s, 1H), 9.05 (s, 1H), 8.33 (s, 1H), 8.14 (s, 1H), 8.06 (s, 1H), 7.91 (s, 1H), 7.83-7.87 (m, 2H), 7.68 (d, J=8.8 Hz, 2H), 7.48 (d, J=8.8 Hz, 2H), 7.11-7.20 (m, 2H), 6.94 (td, J=7.4, 1.2 Hz, 1H), 6.83 (s, 2H), 3.93 (s, 3H), 2.25 (s, 3H).

### EXAMPLE 66A

This example was prepared by substituting (3-aminophenyl)carbamic acid tert-butyl ester for (4-aminophenyl)carbamic acid tert-butyl ester in EXAMPLE 45B.

### EXAMPLE 66B

This example was prepared by substituting EXAMPLE 66A and isocyanatobenzene for EXAMPLE 45C and 1-isocyanato-3-methylbenzene, respectively, in EXAMPLE 45D. ¹H NMR (300 MHz, DMSO-d₆) δ 10.79 (s, 1H), 8.77 (s, 1H), 8.60 (s, 1H), 8.36 (s, 1H), 8.14 (s, 1H), 8.07 (s, 1H), 7.99 (s, 1H), 7.87 (s, 1H), 7.43-7.49 (m, 2H), 7.24-7.38 (m, 5H), 6.97 (t, J=7.3 Hz, 1H), 6.80 (s, 2H), 3.93 (s, 3H).

### EXAMPLE 67

This example was prepared by substituting EXAMPLE 66A and 1-isocyanato-2-methylbenzene for EXAMPLE 45C and 1-isocyanato-3-methylbenzene, respectively, in EXAMPLE 45D. ¹H NMR (300 MHz, DMSO-d₆) δ 10.81 (s, 1H), 9.15 (s, 1H), 8.37 (s, 1H), 8.14 (s, 1H), 8.07 (s, 1H), 8.00-8.03 (m, 1H), 7.86-7.90 (m, 3H), 7.31-7.38 (m, 1H), 7.25-7.31 (m, 2H), 7.11-7.20 (m, 2H), 6.94 (td, J=7.4, 1.2Hz, 1H), 6.80 (s, 2H), 3.93 (s, 3H), 2.26 (s, 3H).

### EXAMPLE 68

This example was prepared by substituting EXAMPLE 66A and I -isocyanato-4-methylbenzene for EXAMPLE 45C and 1-isocyanato-3-methylbenzene, respectively, in EXAMPLE 45D. ¹H NMR (300 MHz, DMSO-d₆) δ 10.78 (s, 1H), 8.73 (s, 1H), 8.49 (s, 1H), 8.36 (s, 1H), 8.14 (s, 1H), 8.06 (s, 1H), 7.96-7.98 (m, 1H), 7.87 (d, J=0.7 Hz, 1H), 7.26-7.37 (m, 5H), 7.09 (d, J=8.5 Hz, 2H), 6.80 (s, 2H), 3.93 (s, 3H), 2.24 (s, 3H).

### EXAMPLE 69

This example was prepared by substituting EXAMPLE 66A for EXAMPLE 45C in EXAMPLE 45D. ¹H NMR (300 MHz, DMSO-d₆) δ 10.79 (s, 1H), 8.80 (s, 1H), 8.55 (s, 1H), 8.36 (s, 1H), 8.14 (s, 1H), 8.06 (s, 1H), 7.99 (s, 1H), 7.87 (s, 1H), 7.26-7.38 (m, 4H), 7.20-7.26 (m, 1H), 7.15 (t, J=7.6 Hz, 1H), 6.76-6.82 (m, 3H), 3.93 (s, 3H), 2.28 (s, 3H).

### EXAMPLE 70

This example was prepared by substituting EXAMPLE 45A and N-(3-aminophenyl)benzamide for EXAMPLE 1B and EXAMPLE 1C, respectively, in EXAMPLE 1D and substituting the product therefrom for EXAMPLE 45B in EXAMPLE 45C. ¹H NMR (300 MHz, DMSO-d₆) δ 10.86 (s, 1H), 10.35 (s, 1H), 8.36-8.39 (m, 2H), 8.14 (s, 1H), 8.07 (s, 1H), 7.98 (dd, J=8.3, 1.5 Hz, 2H), 7.87 (d, J=1.0 Hz, 1H), 7.50-7.63 (m, 4H), 7.44-7.48 (m, 1H), 7.35 (t, J=8.0 Hz, 1H), 6.82 (s, 2H), 3.93 (s, 3H).

### EXAMPLE 71A

This example was prepared as described in EXAMPLE 1D by substituting EXAMPLE 45A and tert-butyl 4-aminopiperidine-1-carboxylate for EXAMPLE 1B and EXAMPLE 1C, respectively, and substituting the product therefrom for EXAMPLE 45B in EXAMPLE 45C. The Boc group was removed with TFA as described in EXAMPLE 1C.

### EXAMPLE 71B

This example was prepared by substituting EXAMPLE 71A and isocyanatobenzene for 45C and 1-isocyanato-3-methylbenzene, respectively, in EXAMPLE 45D. ¹H NMR (300 MHz, DMSO-d₆) δ 8.84 (d, J=7.5 Hz, 1H), 8.54 (s, 1H), 8.12 (s, 1H), 8.11 (s, 1H), 8.02 (s, 1H), 7.84 (d, J=1.0 Hz, 1H), 7.46 (d, J=7.8 Hz, 2H), 7.23 (t, J=8.0 Hz, 2H), 6.97 (s, 2H), 6.93 (t, J=7.3 Hz, 1H), 3.98-4.17 (m, 3H), 3.92 (s, 3H), 2.97 (t, J=11.5 Hz, 2H), 1.89 (dd, J=12.7, 3.2 Hz, 2H), 1.44-1.59 (m, 2H).

### EXAMPLE 72

A mixture of EXAMPLE 71A (75 mg), benzoic acid (26 mg), HoBT (57 mg) and NMM (0.23 mL) in DMF (2 mL) at 0° C. was treated with EDCI (80 mg), allowed to warm to a room temperature, stirred for 5 hours, diluted with water and extracted with ethyl acetate. The extract was dried (Na₂SO₄), filtered and concentrated. The concantrate was triturated with dichloromethane, filtered and air dried. ¹H NMR (300 MHz, DMSO-d₆) δ 8.86 (d, J=7.5 Hz, 1H), 8.12 (s, 1H), 8.11 (d, J=0.7 Hz, 1H), 8.02 (s, 1H), 7.84 (d, J=1.0 Hz, 1H), 7.44-7.49 (m, 3H), 7.36-7.41 (m, 2H), 6.95 (s, 2H), 4.33-4.52 (brm, 1H), 4.04-4.17 (brm, 1H), 3.92 (s, 3H), 3.52-3.71 (brm, 1H), 2.94-3.27 (brm, 2H), 1.77-2.03 (brm, 2H), 1.39-1.64 (brm, 2H).

### EXAMPLE 73A

This example was prepared by substituting tert-butyl trans-4-aminocyclohexylcarbamate for EXAMPLE 71A in EXAMPLE 72 and removing the Boc with TFA as described in EXAMPLE 1C.

### EXAMPLE 73B

This example was prepared by coupling EXAMPLES 45A and 73A as described in EXAMPLE 72 and substituting the product therefrom for EXAMPLE 45B in EXAMPLE 45C. ¹H NMR (300 MHz, DMSO-d₆) δ 8.80 (d, J=7.8 Hz, 1H), 8.27 (d, J=8.1 Hz, 1H), 8.11 (s, 1H), 8.09 (s, 1H), 8.02 (s, 1H), 7.82-7.88 (m, 3H), 7.42-7.55 (m, 3H), 6.97 (s, 2H), 3.92 (s, 3H), 3.73-3.85 (brm, 2H), 1.89-2.01 (brm, 4H), 1.39-1.57 (m, 4H).

### EXAMPLE 74A

An ice cold solution of tert-butyl trans-4-aminocyclohexylcarbamate (250 mg) and isocyanatobenzene (0.11 mL) in DMF (5 mL) was treated with NMM (0.22 mL), stirred at ambient temperature for 5 hours, diluted with water and filtered. The filtrate was dissolved in dichloromethane (10 mL) and treated with TFA (1 mL). The mixture was stirred at ambient temperature for 3 hours and concentrated.

### EXAMPLE 74B

This example was prepared by coupling EXAMPLES 45A and 74A as described in EXAMPLE 72 and substituting the product therefrom for EXAMPLE 45B in EXAMPLE 45C. ¹H NMR (300 MHz, DMSO-d₆) δ 8.77 (d, J=7.8 Hz, 1H), 8.32 (s, 1H), 8.11 (s, 1H), 8.08 (s, 1H), 8.02 (s, 1H), 7.83 (s, 1H), 7.37 (d, J=7.5 Hz, 2H), 7.17-7.25 (m, 2H), 6.96 (s, 2H), 6.88 (t, J=7.3 Hz, 1H), 6.10 (d, J=7.5 Hz, 1H), 3.92 (s, 3H), 3.74-3.87 (brm, 1H), 3.38-3.49 (brm, 1H), 1.89-1.99 (brm, 4H), 1.21-1.54 (m, 4H).

### EXAMPLE 75

This example was prepared by substituting 1-fluoro-2-isocyanatobenzene for isocyanatobenzene in EXAMPLE 74A then EXAMPLE 74B. ¹H NMR (300 MHz, DMSO-d₆) δ 8.77 (d, J=7.8 Hz, 1H), 8.09-8.17 (m, 3H), 8.08 (s, 1H), 8.02 (s, 1H), 7.83 (s, 1H), 7.16 (ddd, J=11.8, 8.1, 1.5 Hz, 1H), 7.07 (t, J=7.1 Hz, 1H), 6.96 (s, 2H), 6.87-6.95 (m, 1H), 6.62 (d, J=7.5 Hz, 1H), 3.92 (s, 3H), 3.75-3.88 (m, 1H), 3.39-3.51 (m, 1H), 1.88-2.02 (m, 4H), 1.37-1.56 (m, 2H), 1.20-1.36 (m, 2H).

### EXAMPLE 76

This example was prepared by substituting tert-butyl 4-aminobenzylcarbamate for tert-butyl trans-4-aminocyclohexylcarbamate in EXAMPLES 74A and 74B. ¹H NMR (300 MHz, DMSO-d₆) δ 9.42 (t, J=5.8 Hz, 1H), 8.71 (s, 1H), 8.70 (s, 1H), 8.16 (s, 1H), 8.11 (d, J=0.7 Hz, 1H), 8.02 (s, 1H), 7.84 (d, J=0.7 Hz, 1H), 7.41-7.47 (m, 4H), 7.24-7.31 (m, 4H), 7.02 (s, 2H), 6.92-6.99 (m, 1H), 4.45 (d, J=5.8Hz, 2H), 3.92 (s, 3H).

### EXAMPLE 77

This example was prepared by substituting tert-butyl 3-aminobenzylcarbamate for tert-butyl trans-4-aminocyclohexylcarbamate in EXAMPLES 74A and 74B. ¹H NMR (300 MHz, DMSO-d₆) δ 9.48 (t, J=5.9 Hz, 1H), 8.68 (s, 1H), 8.62 (s, 1H), 8.19 (s, 1H), 8.11 (s, 1H), 8.02 (s, 1H), 7.84 (s, 1H), 7.36-7.47 (m, 4H), 7.23-7.30 (m, 3H), 7.03 (s, 2H), 6.93-7.01 (m, 2H), 4.49 (d, J=5.8 Hz, 2H), 3.92 (s, 3H).

### EXAMPLE 78A

This example was prepared by substituting 3-bromo-7-iodothieno[3,2-c]pyridin-4-amine for EXAMPLE 45B in EXAMPLE 45C and substituting the product therefrom for 3-bromo-thieno[3,2-c]pyridn-4-ylamine in EXAMPLES 40A and 40B.

### EXAMPLE 78B

This example was prepared by coupling EXAMPLE 78A and 4-(aminomethyl)-N-phenylpiperidine-1-carboxamide (prepared by substituting tert-butyl piperidin-4-ylmethylcarbamate for tert-butyl trans-4-aminocyclohexylcarbamate in EXAMPLE 74A) as described in EXAMPLE 72. ¹H NMR (300 MHz, DMSO-d₆) δ 8.97 (t, J=5.6Hz, 1H), 8.45 (s, 1H), 8.12 (s, 1H), 8.11 (s, 1H), 8.02 (s, 1H), 7.84 (s, 1H), 7.45 (d, J=7.8 Hz, 2H), 7.18-7.25 (m, 2H), 6.99 (s, 2H), 6.91 (t, J=7.3 Hz, 1H), 4.14 (d, J=12.9 Hz, 2H), 3.92 (s, 3H), 3.23 (t, J=5.9 Hz, 2H), 2.79 (t, J=11.9 Hz, 2H), 1.72-1.86 (m, 3H), 1.08-1.24 (m, 2H).

### EXAMPLE 79

This example was prepared by substituting 78A and tert-butyl 4-aminobenzylcarbamate for benzoic acid and 71A, respectively, in EXAMPLE 72, removing the Boc group with TFA as described in EXAMPLE 1C and substituting the product therefrom for tert-butyl trans-4-aminocyclohexylcarbamate in EXAMPLE 74A. ¹H NMR (300 MHz, DMSO-d₆) δ 10.78 (s, 1H), 8.53 (s, 1H), 8.35 (s, 1H), 8.14 (s, 1H), 8.06 (s, 1H), 7.86 (s, 1H), 7.71 (d, J=7.8 Hz, 2H), 7.39-7.43 (m, 2H), 7.32 (d, J=8.1 Hz, 2H), 7.19-7.25 (m, 2H), 6.87-6.93 (m, 1H), 6.80 (s, 2H), 6.59 (t, J=6.4 Hz, 1H), 4.29 (d, J=5.8 Hz, 2H), 3.93 (s, 3H).

### EXAMPLE 80

This example was prepared by substituting EXAMPLE 78A and tert-butyl 3-aminobenzylcarbamate for benzoic acid and EXAMPLE 71A, respectively, in EXAMPLE 72, removing the Boc group with TFA as described in EXAMPLE 1C and substituting the product therefrom for tert-butyl trans-4-aminocyclohexylcarbamate in EXAMPLE 74A. ¹H NMR (300 MHz, DMSO-d₆) δ 10.81 (s, 1H), 8.56 (s, 1H), 8.34 (s, 1H), 8.13 (d, J=0.7 Hz, 1H), 8.06 (s, 1H), 7.86 (d, J=0.7 Hz, 1H), 7.72-7.74 (m, 1H), 7.62-7.66 (m, 1H), 7.39-7.43 (m, 2H), 7.35 (t, J=8.0 Hz, 1H), 7.18-7.25 (m, 2H), 7.10 (d, J=7.8 Hz, 1H), 6.89 (t, J=7.3 Hz, 1H), 6.78 (s, 2H), 6.64 (t, J=5.9 Hz, 1H), 4.32 (d, J=6.4Hz, 2H), 3.93 (s, 3H).

### EXAMPLE 81A

A mixture of (1S,4S)-4-(tert-butoxycarbonylamino)cyclohexanecarboxylic acid (245 mg) in toluene (10 mL) was treated with triethylamine (0.14 nmL) and DPPA (0.22 mL), heated at 70° C. for 45 minutes, cooled to ambient temperature and treated with aniline (0.188 mL). The mixture was stirred overnight at ambient temperature, diluted with ether and washed with 0.5 N HCl, saturated NaHCO₃, water and brine and dried (Na₂SO₄), filtered and concentrated. The concentrate was purified by silica gel chromatography to provide tert-butyl (1S,4S)-4-(3-phenylureido)cyclohexylcarbamate which was dissolved in dichloromethane (2 mL) and TFA (2 mL), stirred at ambient temperature for 12 hours and concentrated.

### EXAMPLE 81B

This example was prepared by substituting EXAMPLE 78A and EXAMPLE 81A for benzoic acid and EXAMPLE 71A respectively, in EXAMPLE 72. ¹H NMR (300 MHz, DMSO-d₆) δ 8.78 (d, J=6.8 Hz, 1H), 8.41 (s, 1H), 8.11 (s, 1H), 8.07 (s, 1H), 8.02 (s, 1H), 7.84 (s, 1H), 7.37 (d, J=7.5 Hz, 2H), 7.21 (t, J=7.8 Hz, 2H), 6.85-6.93 (m, 3H), 6.17 (d, J=6.8 Hz, 1H), 3.92 (s, 3H), 3.83-3.90 (m, 1H), 3.66-3.73 (m, 1H), 1.62-1.79 (m, 8H).

### EXAMPLE 82

This example was prepared by substituting racemic cis-3-(tert-butoxycarbonylamino)cyclohexanecarboxylic acid for cis-4-(tert-butoxycarbonylamino)cyclohexanecarboxylic acid in EXAMPLES 81A and 81B. ¹H NMR (300 MHz, DMSO-d₆) δ 8.81 (d, J=7.8 Hz, 1H), 8.30 (s, 1H), 8.11 (s, 1H), 8.07 (s, 1H), 8.02 (s, 1H), 7.83 (s, 1H), 7.37 (d, J=7.8 Hz, 2H), 7.21 (t, J=7.8 Hz, 2H), 6.93 (s, 2H), 6.88 (t, J=7.1 Hz, 1H), 6.16 (d, J=7.8 Hz, 1H), 3.91 (s, 3H), 3.78-3.94 (m, 1H), 3.47-3.62 (m, 1H), 2.11-2.20 (m, 1H), 1.73-1.92 (m, 3H), 1.00-1.48 (m, 4H).

### EXAMPLE 83A

### (±)(1R,3S)-3-amino-N-phenylcyclohexanecarboxamide

This example was prepared by substituting (±)-(1R,3S)-3-(tert-butoxycarbonylamino)cyclohexanecarboxylic acid and aniline for benzoic acid and EXAMPLE 71A respectively, in EXAMPLE 72 and removing the Boc with TFA as described in EXAMPLE 1C.

### EXAMPLE 83B

This example was prepared by substituting EXAMPLE 78A and EXAMPLE 83A for benzoic acid and EXAMPLE 71A respectively, in EXAMPLE 72. ¹H NMR (300 MHz, DMSO-d₆) δ 9.91 (s, 1H), 8.85 (d, J=7.8 Hz, 1H), 8.11 (s, 1H), 8.09 (s, 1H), 8.01 (s, 1H), 7.83 (s, 1H), 7.60 (d, J=7.8 Hz, 2H), 7.28 (t, J=8.0 Hz, 2H), 7.02 (t, J=7.3 Hz, 1H), 6.95 (s, 2H), 3.91 (s, 3H), 3.83-3.97 (m, 1H), 2.00-2.09 (m, 1H), 1.77-1.96 (m, 3H), 1.13-1.61 (m, 4H).

### EXAMPLE 84A

4-(4,4,5,5-tetramethyl-1,3,2-dioxaborolan-2-yl)-1H-pyrazole (2 g) was added portionwise to an ice cold suspension of NaH (280 mg) in DMF (25 mL). The mixture was stirred at 0° C. for 30 minutes, treated with (2-bromoethoxy)(tert-butyl)diphenylsilane (4.16 g), warmed to 50° C. for 2 hours, quenched with water and extracted with ethyl acetate. The extract was dried (Na₂SO₄), filtered and concentrated, and the concentrate was purified by silica gel chromatography.

### EXAMPLE 84B

This example was prepared by substituting EXAMPLE 78A and tert-butyl 3-aminobenzylcarbamate for benzoic acid and EXAMPLE 71A respectively, in EXAMPLE 72.

### EXAMPLE 84C

This example was prepared by substituting EXAMPLE 84B and EXAMPLE 84A for EXAMPLE 45B and 1-methyl-4-(4,4,5,5-tetramethyl-[ 1,3,2]dioxaborolan-2-yl)-1H-pyrazole in EXAMPLE 45C, removing the Boc with TFA as described in EXAMPLE 1C and substituting the product therefrom for tert-butyl (1R,4R)-4-aminocyclohexylcarbamate in EXAMPLE 74A.

### EXAMPLE 84D

EXAMPLE 84C (0.71 g) and 1M TBAF in THF (1.8 mL) in THF (10 mL) was stirred at ambient temperature for 4 hours, diluted with water and extracted with dichloromethane and methanol. The extract, with the solid at the layer interface, was combined, concentrated and chromatographed on silica gel. The product was further purified by triturating with DMF/water/methanol. ¹H NMR (300 MHz, DMSO-d₆) δ 10.85 (s, 1H), 8.57 (s, 1H), 8.39 (s, 1H), 8.16 (d, J=0.7 Hz, 1H), 8.08 (s, 1H), 7.90 (d, J=0.7 Hz, 1H), 7.71-7.74 (m, 1H), 7.64 (d, J=7.5 Hz, 1H), 7.41 (dd, J=8.6, 1.2 Hz, 2H), 7.35 (t, J=8.0 Hz, 1H), 7.18-7.25 (m, 2H), 7.11 (d, J=7.8 Hz, 1H), 6.94 (s, 2H), 6.89 (t, J=7.3 Hz, 1H), 6.64 (t, J=6.1 Hz, 1H), 4.95 (t, J=5.3 Hz, 1H), 4.32 (d, J=6.1 Hz, 2H), 4.23 (t, J=5.6 Hz, 2H), 3.80 (q, J=5.1 Hz, 2H).

### EXAMPLE 85A

A mixture of EXAMPLE 84D (164 mg) in dimethylacetamide (2.5 mL) was treated with (tBuO)₂PNEt₂ (0.31 mL) and tetrazole (132 mg), stirred at ambient temperature for 2 hours, cooled to −10° C. and treated with 30% H₂O₂ (0.1 mL). The mixture was stirred at ambient temperature for 2.5 hours, treated with of 30% H₂O₂ (0.3 mL), stirred for 3 hours and partitioned between 10% Na₂S₂O₃ and ethyl acetate. The extract was dried (Na₂SO₄), filtered and concentrated, and the concentrate was purified by silica gel chromatography.

### EXAMPLE 85B

A mixture of EXAMPLE 85A (90 mg) in methanol (5 mL) was treated with 4M HCl in dioxane (0.2 mL), stirred for 1 hour, diluted with diethylether and filtered. ¹H NMR (300 MHz, DMSO-d₆) δ 11.09 (s, 1H), 8.90 (s, 1H), 8.90 (s, 2H), 8.76 (s, 1H), 8.33 (d, J=0.7 Hz, 1H), 8.07 (s, 1H), 8.01 (d, J=1.0 Hz, 1H), 7.63-7.68 (m, 2H), 7.35-7.47 (m, 3H), 7.13-7.28 (m, 3H), 6.89 (t, J=7.3 Hz, 1H), 6.74-6.81 (m, 1H), 4.46 (t, J=5.1 Hz, 2H), 4.33 (d, J=4.1 Hz, 2H), 4.20-4.27 (m, 2H).

### EXAMPLE 86

This example was prepared by substituting EXAMPLE 22C and 2-fluoro-1-isocyanato-3-(trifluoromethyl)benzene for EXAMPLE 1B and 1-isocyanato-3-methylbenzene, respectively, in EXAMPLES 1C and 1D, respectively. ¹H NMR (400 MHz, DMSO-d₆) δ 10.65 (s, 1H,) 9.18 (s, 1H), 8.82 (d, J=2.46 Hz, 1H), 8.45 (m, 1H), 8.33 (s, 1H), 8.09 (s, 1H), 7.60 (m, 1H), 7.57 (d, J=8.90 Hz, 2H), 7.47 (d, J=9.21 Hz, 2H), 7.35 (m, 2H), 7.00 (brs, 2H) 3.85 (s, 3H).

### EXAMPLE 87

This example was prepared by substituting EXAMPLE 22C and isocyanatobenzene for EXAMPLE 1B and 1-isocyanato-3-methylbenzene, respectively, in EXAMPLES 1C and 1D, respectively. ¹H NMR (500 MHz, DMSO-d₆) δ 10.64 (s, 1H), 8.69 (s, 1H), 8.64 (s, 1H), 8.33 (s, 1H), 8.09 (s, 1H), 7.60 (s, 1H), 7.54 (d, J=9.15 Hz, 2H), 7.45 (m, 4H), 7.28 (m, 2H), 7.02 (brs, 2H), 6.96 (t, J=7.32 Hz, 1H), 3.85 (s, 3H).

### EXAMPLE 88

This example was prepared as described in EXAMPLES 22B-C by substituting 4,4,5,5-tetramethyl-2-thiophen-3-yl-[1,3,2]dioxaborolane for 1-methyl-4-(4,4,5,5-tetramethyl-[1,3,2]dioxaborolan-2-yl)-1H-pyrazole in EXAMPLE 22B and coupling as described in EXAMPLE 1D but substituting 1-(4-aminophenyl)-3-phenylurea for EXAMPLE 1C. ¹H NMR (400 MHz, DMSO-d₆) δ 10.58 (s, 1H), 8.66 (s, 1H), 8.61 (s, 1H), 8.36 (s, 1H), 7.83 (m, 1H), 7.67 (m, 2H), 7.45 (m, 5H), 7.27 (m, 3H), 7.07 (brs, 2H), 6.96 (t, J=7.36 Hz, 1H).

### EXAMPLE 89

This example was prepared by substituting morpholine for 1-methylpiperazine in EXAMPLE 38A and following the procedures of EXAMPLE 1, but substituting the product therefrom for 5-amino-4-cyano-thiophene-3-carboxylic acid ethyl ester in EXAMPLE 1A and isocyanatobenzene for 1-isocyanato-3-methylbenzene in EXAMPLE 1C. ¹H NMR (500 MHz, DMSO-d₆) δ 10.64 (s, 1H), 8.68 (s, 1H), 8.64 (s, 1H), 8.22 (s, 1H), 7.67 (d, J=8.85 Hz, 2H), 7.50 (brs, 2H), 7.46 (t, J=9.15 Hz, 4H), 7.28 (m, 2H), 6.97 (t, J=7.32 Hz, 1H), 3.70 (m, 4H), 3.10 (m, 4H).

### EXAMPLE 90

This example was prepared as described in EXAMPLE 1 by substituting 3-isocyanatothiophene for 1-isocyanato-3-methylbenzene in EXAMPLE 1C. ¹H NMR (400 MHz, DMSO-d₆) δ 10.63 (s, 1H), 8.89 (s, 1H), 8.63 (s, 1H), 8.37 (s, 1H), 8.32 (s, 1H), 7.98 (brs, 2H), 7.63 (d, J=8.90 Hz, 2H), 7.47 (d, J=8.90 Hz, 2H), 7.43 (m, 1H), 7.28 (dd, J=3.07, 1.23 Hz, 1H), 7.05 (dd, J=4.91, 1.23 Hz, 1H).

### EXAMPLE 91

This example was prepared as described in EXAMPLE 1 by substituting isocyanatocyclopentane for 1-isocyanato-3-methylbenzene in EXAMPLE 1C. ¹H NMR (400 MHz, DMSO-d₆) δ 10.57 (s, 1H), 8.35 (s, 1H), 8.31 (s, 1H), 8.25 (s, 1H), 7.92 (brs, 2H), 7.56 (d, J=8.90 Hz, 2H), 7.38 (d, J=9.21 Hz, 2H), 6.11 (d, J=7.06 Hz, 1H), 3.94 (m, 1H), 1.84 (m, 2H), 1.63 (m, 2H), 1.53 (m, 2H), 1.36 (m, 2H).

### EXAMPLE 92

This example was prepared as described in EXAMPLE 1 by substituting 3-isocyanatopyridine for 1-isocyanato-3-methylbenzene in EXAMPLE 1C. ¹H NMR (400 MHz, DMSO-d₆) δ 10.65 (s, 1H), 8.83 (s, 2H), 8.62 (s, 1H), 8.37 (s, 1H), 8.32 (s, 1H), 8.19 (d, J=3.38 Hz, 1H), 7.95 (m, 1H), 7.96 (brs, 2H), 7.65 (d, J=8.90 Hz, 2H), 7.48 (d, J=8.90 Hz, 2H), 7.32 (m, 1H).

### EXAMPLE 93A

This example was prepared as described in EXAMPLE 1C by substituting 1-isocyanato-4-nitrobenzene and 5-methylisoxazol-3-amine for 1-isocyanato-3-methylbenzene and (4-aminophenyl)carbamic acid tert-butyl ester, respectively, in EXAMPLE 1C.

### EXAMPLE 93B

A mixture of EXAMPLE 93A (700 mg), iron powder (830 mg), NH₄Cl (155 mg) in ethanol (25 mL), THF (28 mL) and water (11 mL) at 85° C. was stirred for 9 hours, cooled to ambient temperature and filtered through diatomaceous earth (CELITE®) with ethanol. The filtrate was concentrated and the concentrate was purified by silica gel chromatography.

### EXAMPLE 93C

This example was prepared by substituting EXAMPLE 93B for EXAMPLE 1C in EXAMPLE 1D. ¹H NMR (400 MHz, DMSO-d₆) δ 10.71 (s, 1H), 9.44 (s, 1H), 8.87 (s, 1H), 8.46 (s, 1H), 8.39 (s, 1H), 8.28 (brs, 2H), 7.65 (d, J=8.90 Hz, 2H), 7.47 (d, J=9.21 Hz, 2H), 6.53 (s, 1H), 2.37 (s, 3H).

### EXAMPLE 94

This example was prepared as described in EXAMPLE 1 by substituting isocyanatocyclopropane for 1-isocyanato-3-methylbenzene in EXAMPLE 1C. ¹H NMR (300 MHz, DMSO-d₆) δ 10.62 (s, 1H), 8.39 (s, 1H), 8.34 (s, 1H), 8.31 (s, 1H), 7.92 (brs, 2H), 7.56 (d, J=9.16 Hz, 2H), 7.41 (d, J=9.16 Hz, 2H), 6.37 (s, 1H), 2.54 (m, 1H), 0.63 (m, 2H), 0.40 (m, 2H).

### EXAMPLE 95

This example was prepared as described in EXAMPLE 1 by substituting 2,4-difluoro-1-isocyanatobenzene for 1-isocyanato-3-methylbenzene in EXAMPLE 1C. ¹H NMR (500 MHz, DMSO-d₆) δ 10.67 (s, 1H), 9.05 (s, 1H), 8.49 (s, 1H), 8.37 (s, 1H), 8.32 (s, 1H), 8.10 (m, 1H), 7.65 (d, J=8.54 Hz, 4H), 7.47 (d, J=8.54 Hz, 2H), 7.31 (t, J=8.85 Hz, 1H), 7.05 (t, J=7.93 Hz, 1H).

### EXAMPLE 96

This example was prepared as described in EXAMPLE 1 by substituting 1,2-difluoro-4-isocyanatobenzene for 1-isocyanato-3-methylbenzene in EXAMPLE 1C. ¹H NMR (500 MHz, DMSO-d₆) δ 10.67 (s, 1H), 8.87 (s, 1H), 8.77 (s, 1H), 8.37 (s, 1H), 8.32 (s, 1H), 7.95 (brs, 2H), 7.68 (m, 1H), 7.64 (d, J=8.85 Hz, 2H), 7.47 (d, J=8.85 Hz, 2H), 7.34 (m, 1H), 7.12 (d, J=9.15 Hz, 1H).

### EXAMPLE 97A

A mixture of 3-(morpholinomethyl)aniline (0.46 g), triethylamine (0.37 mL) and 4-nitrophenylcarbonochloridate (0.53 g) at ambient temperature was stirred for 2 hours, treated with triethylamine (0.37 mL) and tert-butyl 4-aminophenylcarbamate (0.5 g), stirred for 18 hours and partitioned between water and ethyl acetate. The extract was washed with water and brine and dried (Na₂SO₄), filtered and concentrated. The concentrate was purified by silica gel chromatography to provide tert-butyl 4-(3-(3-(morpholinomethyl)phenyl)ureido)phenylcarbamate, which was dissolved in dichloromethane (30 mL), cooled in an ice bath, treated with TFA (1.8 mL), stirred for 30 minutes, warmed at ambient temperature, stirred for 18 hours and concentrated with a toluene/methanol azeotrope.

### EXAMPLE 97B

This example was prepared by substituting EXAMPLE 97A for EXAMPLE 1C in EXAMPLE 1D. ¹H NMR (400 MHz, DMSO-d₆) δ 10.63 (s, 1H), 8.65 (s, 1H), 8.63 (s, 1H), 8.37 (s, 1H), 8.32 (s, 1H), 7.96 (brs, 2H), 7.63 (d, J=8.90 Hz, 2H), 7.47 (d, J=8.90 Hz, 3H), 7.34 (d, J=8.90 Hz, 1H), 7.23 (t, J=7.67 Hz, 1H), 6.92 (d, J=7.36Hz, 1H), 3.59 (m, 4H), 3.47 (s, 2H), 2.40 (s, 4H).

### EXAMPLE 98

This example was prepared by substituting EXAMPLE 74A for EXAMPLE 1C in EXAMPLE 1D. ¹H NMR (400 MHz, DMSO-d₆) δ 9.34 (brs, 1H), 8.83 (d, J=7.67 Hz, 1H), 8.37 (s, 1H), 8.30 (s, 1H), 8.28 (s, 1H), 8.04 (brs, 2H), 7.37 (m, 2H), 7.21 (m, 2H), 6.88 (m, 1H), 6.08 (d, J=6.44 Hz, 1H), 3.44 (brs, 1H), 1.94 (m, 4H), 1.47 (m, 2H), 1.28 (m, 2H).

### EXAMPLE 99

This example was prepared as described in EXAMPLE 93 by substituting 3,5-dimethylisoxazol-4-amine for 5-methylisoxazol-3-amine. ¹H NMR (300 MHz, DMSO-d₆) δ 10.66 (s, 1H), 8.84 (s, 1H), 8.40 (s, 1H), 8.34 (s, 1H), 8.01 (brs, 2H), 7.70 (s, 1H), 7.61 (d, J=9.16 Hz, 2H), 7.46 (d, J=8.82 Hz, 2H), 2.29 (s, 3H), 2.13 (s, 3H).

### EXAMPLE 100

This example was prepared as described in EXAMPLE 93 by substituting thiazol-2-amine for 5-methylisoxazol-3-amine. ¹H NMR (400 MHz, DMSO-d₆) δ 10.67 (s, 1H) 10.46 (s, 1H), 8.98 (s, 1H), 8.38 (s, 1H), 8.32 (s, 1H), 7.98 (brs, 2H), 7.67 (d, J=8.9 Hz, 2H), 7.50 (d, J=8.90 Hz, 2H), 7.37 (d, J=3.68 Hz, 1H), 7.11 (d, J=3.07 Hz, 1H).

### EXAMPLE 101

This example was prepared as described in EXAMPLE 93 by substituting isoxazol-3-amine for 5-methylisoxazol-3-amine. ¹H NMR (500 MHz, DMSO-d₆) δ 10.69 (s, 1H), 9.58 (s, 1H), 8.86 (s, 1H), 8.74 (s, 1H), 8.38 (s, 1H), 8.32 (s, 1H), 7.83 (brs, 2H), 7.67 (d, J=8.24 Hz, 2H), 7.48 (d, J=8.54 Hz, 2H), 6.85 (s, 1H).

### EXAMPLE 102

This example was prepared as described in EXAMPLE 1 by substituting tert-butyl piperidin-4-ylcarbamate and isocyanatobenzene for (4-aminophenyl)carbamic acid tert-butyl ester and 1-isocyanato-3-methylbenzene, respectively, in EXAMPLE 1C. ¹H NMR (400 MHz, DMSO-d₆) δ 9.10 (brs, 1H), 8.85 (d, J=7.67 Hz, 1H), 8.53 (s, 1H), 8.34 (s, 1H), 8.26 (s, 1H), 7.94 (brs, 1H), 7.46 (d, J=7.67 Hz, 2H), 7.22 (m, 2H), 6.93 (t, J=7.36 Hz, 1H), 4.14 (d, J=13.50 Hz, 2H), 4.06 (m, 1H), 2.95 (t, J=11.66 Hz, 2H), 1.88 (d, J=12.27 Hz, 2H), 1.53 (m, 2H).

### EXAMPLE 103

This example was prepared as described in EXAMPLE 1 by substituting (3-aminophenyl)carbamic acid tert-butyl ester and isocyanatobenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.74 (s, 1H), 8.78 (s, 1H), 8.61 (s, 1H), 8.41 (s, 1H), 8.32 (s, 1H), 8.12 (brs, 1H), 7.97 (m, 1H), 7.46 (d, J=7.63 Hz, 2H), 7.34 (m, 1H), 7.29 (t, J=8.24 Hz, 5H), 6.98 (t, J=7.32 Hz, 1H).

### EXAMPLE 104A

A solution of EXAMPLE 1A (750 mg) in THF (34 mL) at −78° C. was treated with 2M LDA in THF (5.1 mL), stirred for 2 hours, treated with iodine (855 mg) in THF (6 mL), stirred for 1 hour, warmed to and at 0° C., stirred for for 2 hours, quenched with saturated NH₄Cl and extracted with ethyl acetate. The extract was washed with 10% Na₂SO₃ and brine and dried (MgSO4), filtered and concentrated. The concentrate was purified by silica gel chromatography.

### EXAMPLE 104B

EXAMPLE 104A (50 mg), 3-methoxyprop-1-yne (0.015 ml), Cl₂Pd(PPh₃)₂ (5 mg), CuI (0.8 mg), triethylamine (0.36 mL) and DMF (0.18 mL) was degassed with nitrogen, heated in a sealed tube at 60° C. for 40 minutes with stirring in a Smith Synthesizer microwave oven (at 200 W). The mixture was partitioned between water and dichloromethane and the extract was washed with brine and dried (MgSO4), filtered and concentrated. The concentrate and was purified by silica gel chromatography.

### EXAMPLE 104C

This example was prepared as described in EXAMPLE 1 by substituting EXAMPLE 104B for EXAMPLE 1A in EXAMPLE 1B and isocyanatobenzene for 1-isocyanato-3-methylbenzene in EXAMPLE 1C. ¹H NMR (500 MHz, DMSO-d₆) δ 10.87 (s, 1H), 8.70 (s, 1H), 8.64 (s, 1H), 8.39 (s, 1H), 7.47 (m, 10H), 6.97 (s, 1H), 4.38 (s, 2H), 3.22 (s, 3H).

### EXAMPLE 105

This example was prepared as described in EXAMPLE 104 by substituting ethynyltrimethylsilane for 3-methoxyprop-1-yne. ¹H NMR (400 MHz, DMSO-d₆) δ 10.84 (s, 1H), 8.71 (s, 1H), 8.64 (s, 1H), 8.40 (s, 1H), 7.65 (d, J=8.90 Hz, 2H), 7.47 (t, J=8.90 Hz, 6H), 7.28 (t, J=8.59,Hz, 2H), 6.97 (t, J=7.36 Hz, 1H), 5.08 (s, 1H).

### EXAMPLE 106

This example was prepared as described in EXAMPLE 104 by substituting 3-ethynylthiophene for 3-methoxyprop-1-yne. ¹H NMR (500 MHz, DMSO-d₆) δ 10.94 (s, 1H), 8.71 (s, 1H), 8.65 (s, 1H), 8.40 (s, 1H), 7.89 (m, 1H), 7.70 (d, J=8.85 Hz, 2H), 7.66 (m, 1H), 7.47 (m, 6H), 7.28 (t, J=7.63 Hz, 2H), 7.10 (d, J=5.19 Hz, 1H), 6.97 (t, J=7.32 Hz, 1H).

### EXAMPLE 107

This example was prepared as described in EXAMPLE 104 by substituting N,N-dimethylprop-2-yn-1-amine for 3-methoxyprop-1-yne. ¹H NMR (400 MHz, DMSO-d₆) δ 10.84 (s, 1H), 8.69 (s, 1H), 8.64 (s, 1H), 8.38 (s, 1H), 7.65 (d, J=9.21 Hz, 2H), 7.46 (m, 6H), 7.27 (t, J=8.59 Hz, 2H), 6.97 (t, J=7.36 Hz, 1H), 3.54 (s, 2H), 2.12 (s, 6H).

### EXAMPLE 108

This example was prepared as described in EXAMPLE 1D by substituting N-(4-aminophenyl)-2-fluorobenzamide for EXAMPLE 1C. ¹H NMR (500 MHz, DMSO-d₆) δ 10.74 (s, 1H), 10.45 (s, 1H), 8.40 (s, 1H), 8.34 (s, 1H), 8.02 (brs, 2H), 7.71 (m, 5H), 7.58 (m, 1H), 7.35 (m, 2H).

### EXAMPLE 109

This example was prepared as described in EXAMPLE 1D by substituting N-(4-aminophenyl)-3-fluorobenzamide for EXAMPLE 1C. ¹H NMR (500 MHz, DMSO-d₆) δ 10.75 (s, 1H), 10.36 (s, 1H), 8.40 (s, 1H), 8.33 (s, 1H), 7.78 (m, 8H), 7.60 (s, 1H), 7.46 (s, 1H).

### EXAMPLE 110

This example was prepared as described in EXAMPLE 1D by substituting N-(4-aminophenyl)-4-fluorobenzamide for EXAMPLE 1C. ¹H NMR (500 MHz, DMSO-d₆) δ 10.74 (s, 1H), 10.30 (s, 1H), 8.41 (s, 1H), 8.34 (s, 1H), 8.05 (m, 2H), 7.90 (brs, 2H), 7.78 (d, J=8.24 Hz, 2H), 7.71 (d, J=8.24 Hz, 2H), 7.37 (t, J=8.85, 8.24 Hz, 1H).

### EXAMPLE 111

This example was prepared as described in EXAMPLE 1D by substituting N-(4-aminophenyl)-2-methylbenzamide for EXAMPLE 1C. ¹H NMR (400 MHz, DMSO-d₆) δ 10.70 (s, 1H), 10.30 (s, 1H), 8.40 (s, 1H), 8.32 (s, 1H), 8.01 (brs, 2H), 7.76 (d, J=8.90 Hz, 2H), 7.68 (d, J=8.90 Hz, 2H), 7.46 (d, J=7.67 Hz, 1H), 7.39 (m, 1H), 7.30 (m, 2H), 2.40 (s, 3H).

### EXAMPLE 112

This example was prepared as described in EXAMPLE 1D by substituting N-(4-aminophenyl)-3-methylbenzamide for EXAMPLE 1C. ¹H NMR (400 MHz, DMSO-d₆) δ 10.71 (s, 1H), 10.22 (s, 1H), 8.40 (s, 1H), 8.33 (s, 1H), 8.03 (brs, 2H), 7.77 (m, 4H), 7.70 (d, J=9.21 Hz, 2H), 7.40 (m, 2H), 2.41 (s, 3H).

### EXAMPLE 113A

A solution of CaCl₂ (104 mg) in ethanol (2.3 mL) was treated with methyl 3-(3-(4-aminophenyl)ureido)benzoate (50 mg) in THF (2.3 mL) and NaBH₄ (71 mg), and the mixture was stirred at reflux for 18 hours, treated with NaBH₄ (280 mg) in 4 portions over 8 hours, cooled to ambient temperature and concentrated. The concentrate was treated with water and washed with dichloromethane. The heterogeneous water layer was filtered, and the solid was collected, washed with water and air dried.

### EXAMPLE 113B

This example was prepared as described in EXAMPLE 1D by substituting EXAMPLE 113A for EXAMPLE 1C. ¹H NMR (500 MHz, DMSO-d₆) δ 10.69 (s, 1H), 8.69 (s, 1H), 8.66 (s, 1H), 8.43 (s, 1H), 8.36 (s, 1H), 7.96 (brs, 2H), 7.63 (d, J=8.85 Hz, 2H), 7.48 (d, J=8.85 Hz, 2H), 7.43 (s, 1H), 7.32 (d, J=8.24 Hz, 1H), 7.22 (t, J=7.93 Hz, 1H), 6.92 (d, J=7.63 Hz, 1H), 4.47 (s, 2H), 3.70 (brs, 1H).

### EXAMPLE 114

This example was prepared by substituting tert-butyl 3-aminobenzylcarbamate for EXAMPLE 1C in EXAMPLE 1D, removing the Boc with TFA as described in EXAMPLE 1C and substituting the product therefrom and isocyanatobenzene for EXAMPLE 45C and 1-isocyanato-3-methylbenzene, respectively, in EXAMPLE 45D. ¹H NMR (500 MHz, DMSO-d₆) δ 10.75 (s, 1H), 8.57 (s, 1H), 8.39 (s, 1H), 8.32 (s, 1H), 7.97 (br s, 2H), 7.69 (s, 1H), 7.62 (d, J=7.32 Hz, 1H), 7.41 (d, J=7.63 Hz, 2H), 7.35 (t, J=7.63 Hz, 1H), 7.22 (t, J=7.32 Hz, 2H), 7.11 (d, J=7.32 Hz, 1H), 6.89 (t, J=6.71 Hz, 1H), 6.64 (s, 1H), 4.32 (d, J=5.19 Hz, 2H).

### EXAMPLE 115

This example was prepared as described in EXAMPLE 1 by substituting (3-aminophenyl)carbamic acid tert-butyl ester and 1-isocyanato-2-methylbenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.74 (s, 1H), 9.15 (s, 1H), 8.41 (s, 1H), 8.33 (s, 1H), 8.00 (s, 1H), 7.88 (m, 4H), 7.30 (m, 3H), 7.16 (m, 2H), 6.95 (t, J=7.32 Hz, 1H), 2.26 (s, 3H).

### EXAMPLE 116

This example was prepared by substituting tert-butyl 3-aminobenzylcarbamate for EXAMPLE 1C in EXAMPLE 1D, removing the Boc with TFA as described in EXAMPLE 1C and substituting the product therefrom for EXAMPLE 45C in EXAMPLE 45D. ¹H NMR (500 MHz, DMSO-d₆) δ 10.75 (s, 1H), 8.48 (s, 1H), 8.39 (s, 1H), 8.33 (s, 1H), 7.90 (br s, 2H), 7.68 (s, 1H), 7.62 (d, J=7.93 Hz, 1H), 7.35 (t, J=7.93 Hz, 1H), 7.25 (s, 1H), 7.18 (d, J=7.93 Hz, 1H), 7.10 (m, 2H), 6.71 (d, J=7.32 Hz, 1H), 6.62 (t, J=5.80 Hz, 1H), 4.32 (d, J=5.80 Hz, 2H), 2.24 (s, 3H).

### EXAMPLE 117

This example was prepared by substituting tert-butyl 3-aminobenzylcarbamate for EXAMPLE 1C in EXAMPLE 1D, removing the Boc with TFA as described in EXAMPLE 1C and substituting the product therefrom and 1-fluoro-3-isocyanatobenzene for EXAMPLE 45C and 1-isocyanato-3-methylbenzene, respectively, in EXAMPLE 45D. ¹H NMR (500 MHz, DMSO-d₆) δ 10.75 (m, 1H), 8.84 (m, 1H), 8.39 (m, 1H), 8.32 (m, 1H), 7.98 (m, 2H), 7.68 (m, 1H), 7.62 (d, J=8.24 Hz, 1H), 7.47 (d, J=12.21 Hz, 1H), 7.35 (t, =7.93 Hz, 1H), 7.24 (m, 1H), 7.11 (d, J=7.63 Hz, 1H), 7.05 (d, J=9.15 Hz, 1H), 6.72 (m, 2H), 4.32 (d, J=6.10 Hz, 2H).

### EXAMPLE 118

This example was prepared as described in EXAMPLE 1 by substituting (3-aminophenyl)carbamic acid tert-butyl ester for (4-aminophenyl)carbamic acid tert-butyl ester in EXAMPLE 1B. ¹H NMR (500 MHz, DMSO-d₆) δ 10.74 (m, 1H), 8.77 (m, 1H), 8.55 (m, 1H), 8.41 (m, 1H), 8.33 (m, 1H), 7.97 (m, 1H), 7.75 (m, 2H), 7.29 (m, 5H), 7.16 (t, J=7.63 Hz, 1H), 6.80 (d, J=7.32 Hz, 1H), 2.28 (s, 3H).

### EXAMPLE 119

This example was prepared as described in EXAMPLE 1 by substituting (3-aminophenyl)carbamic acid tert-butyl ester and 1-isocyanato-4-methylbenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.73 (s, 1H), 8.74 (s, 1H), 8.51 (s, 1H), 8.41 (s, 1H), 8.33 (s, 1H), 7.96 (s, 1H), 7.77 (br s, 2H), 7.30 (m, 5H), 7.09 (m, 2H), 2.24 (s, 3H).

### EXAMPLE 120

This example was prepared as described in EXAMPLE 1 by substituting (3-aminophenyl)carbamic acid tert-butyl ester and 1-fluoro-2-isocyanatobenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.75 (s, 1H), 9.21 (s, 1H), 8.54 (d, J=2.14 Hz, 1H), 8.41 (s, 1H) 8.33 (s, 1H), 8.17 (t, J=8.24 Hz, 1H), 7.98 (s, 1H), 7.73 (br s, 2H), 7.35 (m, 1H), 7.30 (m, 2H), 7.24 (m, 1H), 7.15 (t, J=7.32 Hz, 1H), 7.01 (m, 1H).

### EXAMPLE 121

This example was prepared as described in EXAMPLE 1 by substituting (3-aminophenyl)carbamic acid tert-butyl ester and 1-fluoro-3-isocyanatobenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.75 (s, 1H), 8.85 (s, 2H), 8.41 (s, 1H), 8.32 (s, 1H), 7.97 (s, 1H), 7.96 (br s, 2H), 7.50 (d, J=11.90 Hz, 1H), 7.36 (m, 1H), 7.29 (m, 3H), 7.12 (d, J=7.93 Hz, 1H), 6.79 (t, J=6.10 Hz, 1H).

### EXAMPLE 122

This example was prepared as described in EXAMPLE 1 by substituting (3-aminophenyl)carbamic acid tert-butyl ester and 1-fluoro-4-isocyanatobenzene for (4-aminophenyl)carbamic acid tert-butyl ester and 1-isocyanato-3-methylbenzene, respectively, in EXAMPLE 1B. ¹H NMR (500 MHz, DMSO-d₆) δ 10.73 (m, 1H), 8.78 (m, 1H), 8.65 (m, 1H), 8.40 (m, 1H), 8.33 (m, 1H), 7.97 (m, 1H), 7.78 (m, 2H), 7.47 (m, 2H), 7.33 (m, 1H), 7.28 (m, 2H), 7.13 (t, J=8.85 Hz, 2H).

### EXAMPLE 123

This example was prepared by substituting tert-butyl 3-aminobenzylcarbamate for EXAMPLE 1C in EXAMPLE 1D, removing the Boc with TFA as described in EXAMPLE 1C and substituting the product therefrom and 1-isocyanato-3-(trifluoromethyl)benzene for EXAMPLE 45C and 1-isocyanato-3-methylbenzene, respectively, in EXAMPLE 45D. ¹H NMR (500 MHz, DMSO-d₆) δ 10.75 (s, 1H), 9.04 (s, 1H), 8.39 (s, 1H), 8.32 (s, 1H), 7.97 (br s, 2H), 7.63 (m, 6H), 7.35 (s, 1H), 7.11 (s, 1H), 6.82 (s, 1H), 4.34 (s, 2H).

### EXAMPLE 124

This example was prepared by substituting 2-(2-aminothiazol-5-yl)-N-(3-fluorophenyl)acetamide for EXAMPLE 1C in EXAMPLE 1D. ¹H NMR (500 MHz, DMSO-d₆) δ 12.95 (br s, 1H), 10.47 (s, 1H), 8.61 (s, 1H), 8.32 (s, 1H), 7.97 (br s, 2H), 7.61 (d, J=11.90 Hz, I1H), 7.44 (s, 1H), 7.34 (m, 2H), 6.90 (t, J=6.41 Hz, 1H), 3.91 (s, 2H).

### EXAMPLE 125A

A solution of 4-amino-N-methoxythieno[2,3-d]pyrimidine-5-carboxamide (3 mmol) (prepared by substituting O-methylhydroxylamine for EXAMPLE 1C in EXAMPLE 1D) in THF (12 mL) was added to a suspension of LAH (235 mg) in THF (12 mL) at −78° C. The mixture was stirred for 30 minutes, treated sequentially with water (0.24 mL), 1M NaOH (0.24 mL) and water, (0.72 mL), filtered through diatomaceous earth (CELITE®) and concentrated.

### EXAMPLE 125B

A solution of (4-nitrobenzyl)triphenylphosphonium bromide (1.66 g) in THF (20 mL) at 0° C. was treated with 1.6M n-butyllithium in hexanes (2.2 mL), stirred for 40 minutes, treated with EXAMPLE 125A in THF (20 mL), stirred at 0° C. for 3 hours and at ambient temperature for 18 hours, treated with 5% methanol in dichloromethane, washed with water and dried (Na₂SO₄), filtered and concentrated. The concentrate was triturated with methanol and air dried.

### EXAMPLE 125C

This example was prepared as described for EXAMPLE 93B and substituting EXAMPLE 125B for EXAMPLE 93A.

### EXAMPLE 125D

A mixture of EXAMPLE 125C (110 mg) and 5% Pd on carbon (50 mg) in methanol (10 mL) was shaken under hydrogen (60 psi) at 50° C. for 40 hours, filtered and concentrated.

### EXAMPLE 125E

This example was prepared as described for EXAMPLE 45D and substituting EXAMPLE 125D and isocyanatobenzene for EXAMPLE 45C and 1-isocyanato-3-methylbenzene, respectively. ¹H NMR (400 MHz, DMSO-d₆) δ 8.65 (s, 1H), 8.61 (s, 1H), 8.25 (s, 1H), 7.44 (d, J=7.98 Hz, 2H), 7.35 (d, J=8.59 Hz, 2H), 7.27 (t, J=7.98 Hz, 2H), 7.15 (d, J=8.29 Hz, 2H), 7.10 (s, 1H), 6.99 (s, 2H), 6.95 (m, 1H), 3.22 (t, J=8.29 Hz, 2H), 2.90 (t, J=8.29 Hz, 2H).

### EXAMPLE 126

This example was prepared by substituting 1-(4-aminophenyl)-3-(3-(3-hydroxypropoxy)phenyl)urea for EXAMPLE 1C in EXAMPLE 1D. ¹H NMR (500 MHz, DMSO-d₆) δ 10.68 (s, 1H), 8.70 (s, 1H), 8.66 (s, 1H), 8.48 (br s, 1H), 8.41 (s, 1H), 8.36 (s, 1H), 7.95 (br s, 1H), 7.63 (d, J=8.85 Hz, 2H), 7.47 (d, J=8.85 Hz, 2H), 7.22 (m, 1H), 7.16 (t, J=8.24 Hz, 1H), 6.89 (d, J=7.93 Hz, 1H), 6.54 (m, 1H), 4.00 (t, J=6.41 Hz, 2H), 3.56 (t, J=6.41 Hz, 2H), 1.86 (m, 2H).

Example 127 This example was prepared by substituting tert-butyl 4-(aminomethyl)phenylcarbamate for EXAMPLE 1C in EXAMPLE 1D, removing the Boc with TFA as described in EXAMPLE 1C and substituting the product therefrom and isocyanatobenzene for EXAMPLE 45C and 1-isocyanato-3-methylbenzene, respectively, in EXAMPLE 45D. ¹H NMR (400 MHz, DMSO-d₆) δ 9.50 (t, J=6.14Hz, 1H), 9.06 (s, 1H), 8.68 (s, 1H), 8.66 (s, 1H), 8.31 (s, 1H), 8.27 (s, 1H), 7.81 (br s, 1H), 7.43 (m, 4H), 7.27 (m, 4H), 6.96 (t, J=7.36 Hz, 1H), 4.44 (d, J=5.83 Hz, 2H).

### EXAMPLE 128

This example was prepared by substituting tert-butyl 4-(aminomethyl)phenylcarbamate for EXAMPLE 1C in EXAMPLE 1D, removing the Boc with TFA as described in EXAMPLE 1C and substituting the product therefrom for EXAMPLE 45C in EXAMPLE 45D. ¹H NMR (400 MHz, DMSO-d₆) δ 9.52 (t, J=5.83 Hz, 1H), 9.21 (br s, 1H), 8.67 (s, 1H) 8.59 (s, 1H), 8.34 (s, 1H), 8.30 (s, 1H), 7.92 )br s, 1H), 7.43 (d, J=8.59 Hz, 2H), 7.29 (d, J=5.83 Hz, 2H), 7.25 (s, 1H), 7.21 (d, J=8.59 Hz, 1H), 7.14 (m, 1H), 6.78 (d, J=7.98 Hz, 1H), 4.44 (d, J=5.83 Hz, 2H), 2.27 (s, 3H).

### EXAMPLE 129

This example was prepared by substituting tert-butyl 4-(aminomethyl)phenylcarbamate for EXAMPLE 1C in EXAMPLE 1D, removing the Boc with TFA as described in EXAMPLE 1C and substituting the product therefrom and 1-fluoro-3-isocyanatobenzene for EXAMPLE 45C and 1-isocyanato-3-methylbenzene, respectively, in EXAMPLE 45D. ¹H NMR (400 MHz, DMSO-d₆) δ 9.52 (t, J=5.83 Hz, 1H), 9.18 (brs, 1H), 8.95 (s, 1H), 8.80 (s, 1H), 8.34 (s, 1H), 8.30 (s, 1H), 7.91 (br s, 1H), 7.49 (m, 1H), 7.43 (d, J=8.59 Hz, 2H), 7.29 (m, 3H), 7.11 (m, 1H), 6.77 (s, 1H), 4.45 (d, J=5.83 Hz, 2H).

The foregoing is meant to illustrate the invention but not to limit it. Variations and changes obvious to one skilled in the art are intended to be within the scope of the invention as defined in the claims.

We claim:

1. A compound having formula (I) 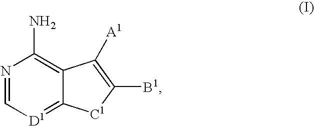
or a therapeutically acceptable salt thereof, wherein
A¹ is C(O)NHR¹, C(O)N(R¹)₂, NHC(O)R¹, NR¹C(O)R¹, NHC(O)NHR¹, NHC(O)N(R¹)₂, NR¹C(O)NHR¹, NR¹C(O)N(R¹)₂, SO₂NHR¹, SO₂N(R¹)₂, NHSO₂R¹, NR¹SO₂R¹, OC(O)OR¹, NHC(O)OR¹, NR¹C(O)OR¹ or R⁵;
R¹ is R², R³, R⁴ or R⁵;
R² is phenyl which is unfused or fused with benzene, heteroarene or R^{2A}; R^{2A} is cycloalkane, cycloalkene, heterocycloalkane or heterocycloalkene;
R³ is heteroaryl which is unfused or fused with benzene, heteroarene or R^{3A}; R^{3A} is cycloalkane, cycloalkene, heterocycloalkane or heterocycloalkene;
R⁴ is cycloalkyl, cycloalkenyl, heterocycloalkyl or heterocycloalkenyl, each of which is unfused or fused with benzene, heteroarene or R^{4A}; R^{4A} is cycloalkane, cycloalkene, heterocycloalkane or heterocycloalkene;
R⁵ is alkyl, alkenyl or alkynyl, each of which is substituted with one or two of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHSO₂R⁶, NR⁶SO₂R⁶, NHC(O)OR⁶, NR⁶C(O)OR⁶, SO₂NH₂, SO₂NHR⁶, SO₂N(R⁶)₂, NHC(O)NH₂, NHC(O)NHR⁶, NHC(O)N(R⁶)₂, NR⁶C(O)N(R⁶)₂, OH, (O), C(O)OH, CN, NH₂, CF₃, OCF₃, CF₂CF₃, F, Cl, Br or I;
R⁶ is R⁷, R⁸ or R⁹;
R⁷ is phenyl which is unfused or fused with benzene, heteroarene or R^{7A}; R^{7A} is cycloalkane, cycloalkene, heterocycloalkane or heterocycloalkene;
R⁸ is heteroaryl which is unfused or fused with benzene, heteroarene or R^{8A}; R^{8A} is cycloalkane, cycloalkene, heterocycloalkane or heterocycloalkene;
R⁹ is cycloalkyl, cycloalkenyl, heterocycloalkyl or heterocycloalkenyl, each of which is unfused or fused with benzene, heteroarene or R^{9A}; R^{9A} is cycloalkane, cycloalkene, heterocycloalkane or heterocycloalkene;
wherein the moieties represented by R¹, R², R³, R⁴, R⁵ and R⁶ are independently substituted with one or two of independently selected R¹⁰, OR¹⁰, SR¹⁰, S(O)R¹, SO₂R¹⁰, NH₂, NHR¹⁰, N(R¹⁰)₂, C(O)R¹⁰, C(O)OR¹⁰, C(O)NHR¹⁰, C(O)N(R¹⁰)₂, NHC(O)R¹⁰, NR¹⁰C(O)R¹⁰, NHC(O)NHR¹⁰, NHC(O)N(R¹⁰)₂, NR¹⁰C(O)NHR¹⁰, NR¹⁰C(O)N(R¹⁰)₂, SO₂NHR¹⁰, SO₂N(R¹⁰)₂, NHSO₂R¹⁰, NR SO₂R¹⁰, OC(O)OR¹⁰, NHC(O)OR¹⁰ or NR¹C(O)OR¹⁰;
R¹⁰ is R¹¹, R¹², R¹³ or R¹⁴;
R¹¹ is phenyl which is unfused or fused with benzene, heteroarene or R^{11A}; R^{11A} is cycloalkane, cycloalkene, heterocycloalkane or heterocycloalkene;
R¹² is heteroaryl which is unfused or fused with benzene, heteroarene or R^{12A}; R^{12A} is cycloalkane, cycloalkene, heterocycloalkane or heterocycloalkene;
R¹³ is cycloalkyl, cycloalkenyl, heterocycloalkyl or heterocycloalkenyl, each of which is unifused or fused with benzene, heteroarene or R^{13A}; R^{13A} is cycloalkane, cycloalkene, heterocycloalkane or heterocycloalkene;
R¹⁴ is alkyl, alkenyl or alkynyl, each of which is unsubstituted or substituted with one or two of independently selected R¹⁵ or NHC(O)NHR¹⁵;
R¹⁵ is R¹⁶, R¹⁷ R¹⁸;
R¹⁶ is phenyl which is unfused or fused with benzene, heteroarene or R^{6A}; R^{16A} is cycloalkane, cycloalkene, heterocycloalkane or heterocycloalkene;
R¹⁷ is heteroaryl which is unfused or fused with benzene, heteroarene or R^{7A}; R^{17A} is cycloalkane, cycloalkene, heterocycloalkane or heterocycloalkene;
R¹⁸ is cycloalkyl, cycloalkenyl, heterocycloalkyl or heterocycloalkenyl, each of which is unfused or fused with benzene, heteroarene or R^{18A}; R^{18A} is cycloalkane, cycloalkene, heterocycloalkane or heterocycloalkene;
B¹ is H, R¹⁹, C(O)NHR¹⁹, C(O)N(R¹⁹)₂, NHC(O)R¹⁹, NR¹C(O)R¹⁹, NHC(O)NHR¹⁹, NHC(O)N(R¹⁹)₂, NR¹⁹C(O)NHR¹⁹, NR¹C(O)N(R¹⁹)₂, SO₂NHR¹⁹, SO₂N(R¹⁹)₂, NHSO₂R¹⁹, NR¹⁹SO₂R¹⁹, OC(O)OR¹⁹, NHC(O)OR¹⁹, or NR¹⁹C(O)OR¹⁹;
R¹⁹ is R²⁰, R²¹, R²² or R²³;
R²⁰ is phenyl which is unfused or fused with benzene, heteroarene or R^{20A}; R^{20A} is cycloalkane, cycloalkene, heterocycloalkane or heterocycloalkene;
R²¹ is heteroaryl which is unfused or fused with benzene, heteroarene or R^{21A}; R^{21A} is cycloalkane, cycloalkene, heterocycloalkane or heterocycloalkene;
R²² is cycloalkyl, cycloalkenyl, heterocycloalkyl or heterocycloalkenyl, each of which is unfused or fused with benzene, heteroarene or R^{22A}; R^{22A} is cycloalkane, cycloalkene, heterocycloalkane or heterocycloalkene;
R²³ is alkyl, alkenyl or alkynyl, each of which is unsubstituted or substituted with one or two of independently selected R²⁴, OR²⁴, SR²⁴, S(O)R²⁴, SO₂R²⁴, NH₂, NHR²⁴, N(R²⁴)₂, C(O)R²⁴, C(O)NH₂, C(O)NHR²⁴, C(O)N(R²⁴)₂, NHC(O)R²⁴, NR²⁴C(O)R²⁴, NHSO₂R²⁴, NR²⁴SO₂R²⁴, NHC(O)OR²⁴, NR²⁴C(O)OR²⁴, SO₂NH₂, SO₂NHR²⁴, SO₂N(R²⁴)₂, NHC(O)NH₂, NHC(O)NHR²⁴, NHC(O)N(R²⁴)₂, NR²⁴C(O)N(R²⁴)₂, OH, (O), C(O)OH, CN, NH₂, CF₃, OCF₃, CF₂CF₃, F, Cl, Br or I;
R²⁴ is R²⁵, R²⁶, R²⁷, alkyl, alkenyl or alkynyl;
R²⁵ is phenyl which is unfused or fused with benzene, heteroarene or R^{25A}; R^{25A} is cycloalkane, cycloalkene, heterocycloalkane or heterocycloalkene;
R²⁶ is heteroaryl which is unfused or fused with benzene, heteroarene or R^{26A}; R^{26A} is cycloalkane, cycloalkene, heterocycloalkane or heterocycloalkene;
R²⁷ is cycloalkyl, cycloalkenyl, heterocycloalkyl or heterocycloalkenyl, each of which is unfused or fused with benzene, heteroarene or R^{27A}; R^{27A} is cycloalkane, cycloalkene, heterocycloalkane or heterocycloalkene;
C¹ is O, S, S(O), SO₂, NH, or N(C²);
C² is R²⁸, R²⁹, R³⁰ or R³¹;
R²⁸ is phenyl which is unfused or fused with benzene, heteroarene or R^{28A}; R^{28A} is cycloalkane, cycloalkene, heterocycloalkane or heterocycloalkene;
R²⁹ is heteroaryl which is unfused or fused with benzene, heteroarene or R^{29A}; R^{29A} is cycloalkane, cycloalkene, heterocycloalkane or heterocycloalkene;
R³⁰ is cycloalkyl, cycloalkenyl, heterocycloalkyl or heterocycloallkcnyl, each of which is unfused or fused with benzene, heteroarene or R^{30A}; R^{30A} is cycloalkane, cycloalkene, heterocycloalkane or heterocycloalkene;
R³¹ is alkyl, alkenyl or alkynyl, each of which is substituted with one or two of independently selected R³², OR³²SR³², S(O)R³², SO₂R³², NH₂, NHR³², N(R³²)₂, C(O)R³², C(O)NH₂, C(O)NHR³², C(O)N(R³²)₂, NHC(O)R³², NR³²C(O)R³², NHSO₂R³², NR³²SO₂R³², NHC(O)OR³², NR³²C(O)OR³², SO₂NH₂, SO₂NHR³², SO₂N(R³²)₂, NHC(O)NH₂, NHC(O)NHR³², NHC(O)N(R³²)₂, NR³²C(O)N(R³²)₂, OH, (O), C(O)OH, CN, NH₂, CF₃, OCF₃, CF₂CF₃, F, Cl, Br or I;
C³² is R³³, R³⁴ or R³⁵;
R³³ is phenyl which is unfused or fused with benzene, heteroarene or R^{33A}; R^{33A} is cycloalkane, cycloalkene, heterocycloalkane or heterocycloalkene;
R³⁴ is heteroaryl which is unfused or fused with benzene, heteroarene or R^{34A}; R^{34A} is cycloalkane, cycloalkene, heterocycloalkane or heterocycloalkene;
R³⁵ is cycloalkyl, cycloalkenyl, heterocycloalkyl or heterocycloalkenyl, each of which is unfused or fused with benzene, heteroarene or R^{35A}; R^{35A} is cycloalkane, cycloalkene, heterocycloalkane or heterocycloalkene;
D¹ is N, CH or C(D²);
D² is R³⁶, R³⁷, R³⁸ or R³⁹;
R³⁶ is phenyl which is unfused or fused with benzene, heteroarene or R^{36A}; R^{36A} is cycloalkane, cycloalkene, heterocycloalkane or heterocycloalkene;
R³⁷ is heteroaryl which is unfused or fused with benzene, heteroarene or R^{37A}; R^{37A} is cycloalkane, cycloalkene, heterocycloalkane or heterocycloalkene;
R³⁸ is cycloalkyl, cycloalkenyl, heterocycloalkyl or heterocycloalkenyl, each of which is unfused or fused with benzene, heteroarene or R^{38A}; R^{38A} is cycloalkane, cycloalkene, heterocycloalkane or heterocycloalkene;
R³⁹ is alkyl, alkenyl or alkynyl, each of which is substituted with one or two of independently selected R⁴⁰, OR⁴⁰, SR⁴⁰, S(O)R⁴⁰, SO₂R⁴⁰, NH₂, NHR⁴⁰, N(R⁴⁰)₂, C(O)R⁴⁰, C(O)NH₂, C(O)NHR⁴⁰, C(O)N(R⁴⁰)₂, NHC(O)R⁴⁰, NR⁴⁰C(O)R⁴⁰, NHSO₂R⁴⁰, NR⁴⁰SO₂R⁴⁰, NHC(O)OR⁴⁰, NR⁴⁰C(O)OR⁴⁰, SO₂NH₂, SO₂NHR⁴⁰, SO₂N(R⁴⁰)₂, NHC(O)NH₂, NHC(O)NHR⁴⁰, NHC(O)N(R⁴⁰)₂, NR⁴⁰C(O)N(R⁴⁰)₂, OH, (O), C(O)OH, CN, NH₂, CF₃, OCF₃, CF₂CF₃, F, Cl, Br or I;
R⁴⁰ is R⁴¹, R⁴² or R⁴³;
R⁴¹ is phenyl which is unfused or fused with benzene, heteroarene or R^{41A}; R^{41A} is cycloalkane, cycloalkene, heterocycloalkane or heterocycloalkene;
R⁴² is heteroaryl which is unfused or fused with benzene, heteroarene or R^{42A}; R^{42A} is cycloalkane, cycloalkene, heterocycloalkane or heterocycloalkene;
R⁴³ is cycloalkyl, cycloalkenyl, heterocycloalkyl or heterocycloalkenyl, each of which is unfused or fused with benzene, heteroarene or R^{43A}; R^{43A} is cycloalkane, cycloalkene, heterocycloalkane or heterocycloalkene;
wherein each foregoing cyclic moiety is independently unsubstituted or substituted or further unsubstituted or further substituted with one or two or three or four of independently selected R⁴⁴, OR⁴⁴, SR⁴⁴, S(O)R⁴⁴, SO₂R⁴⁴, NH₂, NHR⁴⁴, N(R⁴⁴)₂, C(O)R⁴⁴, C(O)OR⁴⁴, C(O)NH₂, C(O)NHR⁴⁴, C(O)N(R⁴⁴)₂, NHC(O)R⁴, NR⁴⁴C(O)R⁴⁴, NHSO₂R⁴⁴, NR⁴⁴SO₂R⁴⁴, NHC(O)OR⁴⁴, NR⁴⁴C(O)OR⁴⁴, SO₂NH₂, SO₂NHR⁴⁴, SO₂N(R⁴⁴)₂, NHC(O)NH₂, NHC(O)NHR⁴⁴, NHC(O)N(R⁴⁴)₂, NR⁴⁴C(O)N(R⁴⁴)₂, C(N)NH₂, C(N)NHR⁴⁴, C(N)N(R⁴⁴)₂, NHC(N)NH₂, NHC(N)NHR⁴⁴, NHC(N)N(R⁴⁴)₂, OH, (O), C(O)H, C(O)OH, NO₂, CN, CF₃, OCF₃, CF₂CF₃, F, Cl, Br or I;
R⁴⁴ is R⁴⁵, R⁴⁶, R⁴⁷ or R⁴⁸;
R⁴⁵ is phenyl which is unfused or fused with benzene, heteroarene or R^{45A}; R^{45A} is cycloalkane, cycloalkene, heterocycloalkane or heterocycloalkene;
R⁴⁶ is heteroaryl which is unfused or fused with benzene, heteroarene or R^{46A}; R^{46A} is cycloalkane, cycloalkene, heterocycloalkane or heterocycloalkene;
R⁴⁷ is cycloalkyl, cycloalkenyl, heterocycloalkyl or heterocycloalkenyl, each of which is unfused or fused with benzene, heteroarene or R^{47A}; R^{47A} is cycloalkane, cycloalkene, heterocycloalkane or heterocycloalkene;
R⁴⁸ is alkyl, alkenyl or alkynyl, each of which is substituted with one or two of independently selected R⁴⁹, OR⁴⁹, SR⁴⁹, S(O)R⁴⁹, SO₂R⁴⁹, NH₂, NHR⁴⁹, N(R⁴⁹)₂, C(O)R⁴⁹, C(O)NH₂, C(O)NHR⁴⁹, C(O)N(R⁴⁹)₂, NHC(O)R⁴⁹, NR⁴⁹C(O)R⁴⁹, NHSO₂R⁴⁹, NR⁴⁹SO₂R⁴⁹, NHC(O)OR⁴⁹, NR⁴⁹C(O)OR₄₉, SO₂NH₂, SO₂NHR⁴⁹, SO₂N(R⁴⁹)₂, NHC(O)NH₂, NHC(O)NHR⁴⁹, NHC(O)N(R⁴⁹)₂, NR⁴⁹C(O)N(R⁴⁹)₂, OP(O)(OH)₂, OP(O)(OH)(OR⁴⁴), OP(O)(OR⁴⁴)₂, OH, (O), C(O)OH, CN, CF₃, OCF₃, CF₂CF₃, F, Cl, Br or I;
R⁴⁹ is R⁵⁰, R⁵¹, R⁵², alkyl, alkenyl or alkynyl;
R⁵⁰ is phenyl which is unfused or fused with benzene, heteroarene or R^{50A}; R^{50A} is cycloalkane, cycloalkene, heterocycloalkane or heterocycloalkene;
R⁵¹ is heteroaryl which is unfused or fused with benzene, heteroarene or R^{51A}; R^{51A} is cycloalkane, cycloalkene, heterocycloalkane or heterocycloalkene; and
R⁵² is cycloalkyl, cycloalkenyl, heterocycloalkyl or heterocycloalkenyl, each of which is unfused or fused with benzene, heteroarene or R^{52A}; R^{52A} is cycloalkane, cycloalkene, heterocycloalkane or heterocycloalkene;
wherein the moieties represented by R⁴⁵, R⁴⁶, R⁴⁷ and R⁴⁹ are independently unsubstituted or substituted with one or two or three of four of independently selected alkyl, alkenyl, alkynyl, OH, (O), C(O)OH, CN, CF₃, OCF₃, CF₂CF₃, F, Cl, Br or I.

2. The compound of claim 1, or a therapeutically acceptable salt thereof, wherein A¹ is C(O)NHR¹, C(O)N(R¹)₂, NHC(O)R¹, NR¹C(O)R¹, NHC(O)NHR¹, NHC(O)N(R¹)₂, NR¹C(O)NHR¹, NR¹C(O)N(R¹)₂, SO₂NHR¹, SO₂N(R¹)₂, NHSO₂R¹, NR¹SO₂R¹, OC(O)OR¹, NHC(O)OR¹, NR¹C(O)OR¹ or R⁵;
R¹ is R², R³ or R⁴;
R² is phenyl which is unfused or fused with benzene or heteroarene;
R³ is heteroaryl which is unfused or fused with benzene or heteroarene;
R⁴ is cycloalkyl, cycloalkenyl, heterocycloalkyl or heterocycloalkenyl, each of which is unfused or fused with benzene or heteroarene;
R⁵ is alkyl, alkenyl or alkynyl, each of which is substituted with one or two of independently selected R⁶, OR⁶, SR⁶, S(O)R⁶, SO₂R⁶, NH₂, NHR⁶, N(R⁶)₂, C(O)R⁶, C(O)NH₂, C(O)NHR⁶, C(O)N(R⁶)₂, NHC(O)R⁶, NR⁶C(O)R⁶, NHC(O)NHR⁶, OH, (O), C(O)OH, CN, NH₂, CF₃, OCF₃, CF₂CF₃, F, Cl, Br or I;
R⁶ is R⁷, R⁸ or R⁹;
R⁷ is phenyl which is unfused or fused with benzene or heteroarene;
R⁸ is heteroaryl which is unfused or fused with benzene or heteroarene;
R⁹ is cycloalkyl, cycloalkenyl, heterocycloalkyl or heterocycloalkenyl, each of which is unfused or fused with benzene or heteroarene;
wherein the moieties represented by R¹, R², R³, R⁴, R⁵ and R⁶ are independently substituted with one or two of independently selected R¹⁰, OR¹⁰, SR¹⁰, S(O)R¹⁰, SO₂R¹⁰, NH₂, NHR¹⁰, N(R¹⁰)₂, C(O)R¹⁰, C(O)OR¹⁰, C(O)NHR¹⁰, C(O)N(R¹⁰)₂, NHC(O)R¹⁰, NR¹⁰C(O)R¹⁰ or NHC(O)NHR¹⁰;
R¹⁰ is R¹¹, R¹², R¹³ or R¹⁴;
R¹¹ is phenyl which is unfused or fused with benzene or heteroarene;
R¹² is heteroaryl which is unfused or fused with benzene or heteroarene;
R¹³ is cycloalkyl, cycloalkenyl, heterocycloalkyl or heterocycloalkenyl, each of which is unfused or fused with benzene or heteroarene;
R¹⁴ is alkyl, alkenyl or alkynyl, each of which is unsubstituted or substituted with one or two of independently selected R¹⁵ or NHC(O)NHR¹⁵;
R¹⁵ is R¹⁶, R¹⁷ R¹⁸;
R¹⁶ is phenyl which is unfused or fused with benzene or heteroarene;
R¹⁷ is heteroaryl which is unfused or fused with benzene or heteroarene;
R¹⁸ is cycloalkyl, cycloalkenyl, heterocycloalkyl or heterocycloalkenyl, each of which is unfused or fused with benzene or heteroarene;
B¹ is H or R¹⁹;
R¹⁹ is R²⁰, R²¹, R²² or R²³;
R²⁰ is phenyl which is unfused or fused with benzene or heteroarene;
R²¹ is heteroaryl which is unfused or fused with benzene or heteroarene;
R²² is cycloalkyl, cycloalkenyl, heterocycloalkyl or heterocycloalkenyl, each of which is unfused or fused with benzene or heteroarene;
R²³ is alkyl, alkenyl or alkynyl, each of which is unsubstituted or substituted with one or two of independently selected R²⁴, OR²⁴, N(R²⁴)₂, C(O)N(R²⁴)₂, NHC(O)R²⁴, NR²⁴C(O)R²⁴;
R²⁴ is alkyl, alkenyl or alkynyl;
C¹ is O, S, S(O), SO₂, NH, or N(C²);
C² is R²⁸, R²⁹ or R³⁰;
R²⁸ is phenyl which is unfused or fused with benzene or heteroarene;
R²⁹ is heteroaryl which is unfused or fused with benzene or heteroarene;
R³⁰ is cycloalkyl, cycloalkenyl, heterocycloalkyl or heterocycloalkenyl, each of which is unfused or fused with benzene or heteroarene;
D¹ is N, CH or C(D²);
D² is R³⁶, R³⁷ or R³⁸;
R³⁶ is phenyl which is unfused or fused with benzene or heteroarene;
R³⁷ is heteroaryl which is unfused or fused with benzene or heteroarene;
R³⁸ is cycloalkyl, cycloalkenyl, heterocycloalkyl or heterocycloalkenyl, each of which is unfused or fused with benzene or heteroarene;
wherein each foregoing cyclic moiety is independently unsubstituted or substituted or further unsubstituted or further substituted with one or two or three or four of independently selected R⁴⁴, OR⁴⁴, SR⁴⁴, S(O)R⁴⁴, SO₂R⁴⁴, NH₂, NHR⁴⁴, N(R⁴⁴)₂, C(O)R⁴⁴, C(O)OR⁴⁴, C(O)NH₂, C(O)NHR⁴⁴, C(O)N(R⁴⁴)₂, NHC(O)R⁴⁴, OH, (O), C(O)H, C(O)OH, NO₂, CN, CF₃, OCF₃, CF₂CF₃, F, Cl, Br or I;
R⁴⁴ is R⁴⁵, R⁴⁶, R⁴⁷ or R⁴⁸;
R⁴⁵ is phenyl which is unfused or fused with benzene or heteroarene;
R⁴⁶ is heteroaryl which is unfused or fused with benzene or heteroarene;
R⁴⁷ is cycloalkyl, cycloalkenyl, heterocycloalkyl or heterocycloalkenyl, each of which is unfused or fused with benzene or heteroarene;
R⁴⁸ is alkyl substituted with OP(O)(OH)₂;
wherein the moieties represented by R⁴⁵, R⁴⁶ and R⁴⁷ are independently unsubstituted or substituted with one or two or three of four of independently selected alkyl, alkenyl, alkynyl, OH, (O), C(O)OH, CN, CF₃, OCF₃, CF₂CF₃, F, Cl, Br or I.

3. The compound of claim 2, or a therapeutically acceptable salt thereof, wherein A¹ is C(O)NHR¹ or R⁵;
R¹ is R², R³ or R⁴;
R² is phenyl which is unfused or fused with benzene or heteroarene;
R³ is heteroaryl which is unfused or fused with benzene or heteroarene;
R⁴ is cycloalkyl, cycloalkenyl, heterocycloalkyl or heterocycloalkenyl, each of which is unfused or fused with benzene or heteroarene;
R⁵ is alkyl, alkenyl or alkynyl, each of which is substituted with one or two of independently selected R⁶, NHC(O)NHR⁶;
R⁶ is R⁷, R⁸ or R⁹;
R⁷ is phenyl which is unfused or fused with benzene or heteroarene;
R⁸ is heteroaryl which is unfused or fused with benzene or heteroarene;
R⁹ is cycloalkyl, cycloalkenyl, heterocycloalkyl or heterocycloalkenyl, each of which is unfused or fused with benzene or heteroarene;
wherein the moieties represented by R¹, R², R³, R⁴, R⁵ and R⁶ are independently substituted with one or two of independently selected R¹⁰, OR¹⁰, SR¹⁰, S(O)R¹⁰, SO₂R¹⁰, NH₂, NHC(O)R¹⁰, NHC(O)NHR¹⁰;
R¹⁰ is R¹¹, R¹², R¹³ or R¹⁴;
R¹¹ is phenyl which is unfused or fused with benzene or heteroarene;
R¹² is heteroaryl which is unfused or fused with benzene or heteroarene;
R¹³ is cycloalkyl, cycloalkenyl, heterocycloalkyl or heterocycloalkenyl, each of which is unfused or fused with benzene or heteroarene;
R¹⁴ is alkyl, alkenyl or alkynyl, each of which is unsubstituted or substituted with one or two of independently selected R¹⁵ or NHC(O)NHR¹⁵;
R¹⁵ is R¹⁶, R¹⁷ R¹⁸;
R¹⁶ is phenyl which is unfused or fused with benzene or heteroarene;
R¹⁷ is heteroaryl which is unfused or fused with benzene or heteroarene;
R¹⁸ is cycloalkyl, cycloalkenyl, heterocycloalkyl or heterocycloalkenyl, each of which is unfused or fused with benzene or heteroarene;
B¹ is H or R¹⁹;
R¹⁹ is R²⁰, R²¹, R²² or R²³;
R²⁰ is phenyl which is unfused or fused with benzene or heteroarene;
R²¹ is heteroaryl which is unfused or fused with benzene or heteroarene;
R²² is cycloalkyl, cycloalkenyl, heterocycloalkyl or heterocycloalkenyl, each of which is unfused or fused with benzene or heteroarene;
R²³ is alkyl, alkenyl or alkynyl, each of which is unsubstituted or substituted with one or two of independently selected R²⁴, OR²⁴ or N(R²⁴)₂;
R²⁴ is alkyl, alkenyl or alkynyl;
C¹ is O, S, S(O), SO₂, NH, or N(C²);
C² is R²⁸, R²⁹ or R³⁰;
R²⁸ is phenyl which is unfused or fused with benzene or heteroarene;
R²⁹ is heteroaryl which is unfused or fused with benzene or heteroarene;
R³⁰ is cycloalkyl, cycloalkenyl, heterocycloalkyl or heterocycloalkenyl, each of which is unfused or fused with benzene or heteroarene;
D¹ is N, CH or C(D²);
D² is R³⁶, R³⁷ or R³⁸;
R³⁶ is phenyl which is unfused or fused with benzene or heteroarene;
R³⁷ is heteroaryl which is unfused or fused with benzene or heteroarene;
R³⁸ is cycloalkyl, cycloalkenyl, heterocycloalkyl or heterocycloalkenyl, each of which is unfused or fused with benzene or heteroarene;
wherein each foregoing cyclic moiety is independently unsubstituted or substituted or further unsubstituted or further substituted with one or two or three or four of independently selected R⁴⁴, OR⁴⁴, CN, CF₃, OCF₃, CF₂CF₃, F, Cl, Br or I;
R⁴⁴ is R⁴⁵, R⁴⁶, R⁴⁷ or R⁴⁸;
R⁴⁵ is phenyl which is unfused or fused with benzene or heteroarene;
R⁴⁶ is heteroaryl which is unfused or fused with benzene or heteroarene;
R⁴⁷ is cycloalkyl, cycloalkenyl, heterocycloalkyl or heterocycloalkenyl, each of which is unfused or fused with benzene or heteroarene;
R⁴⁸ is alkyl substituted with OP(O)(OH)₂;
wherein the moieties represented by R⁴⁵, R⁴⁶, and R⁴⁷ are independently unsubstituted or substituted with one or two or three of four of independently selected alkyl.

4. The compound of claim 3, or a therapeutically acceptable salt thereof, wherein A¹ is C(O)NHR¹ or R⁵;
R¹ is R², R³ or R⁴;
R² is phenyl;
R³ is heteroaryl;
R⁴ is cycloalkyl or heterocycloalkyl;
R⁵ is alkyl, alkenyl or alkynyl, each of which is substituted with R⁶, NHC(O)NHR⁶;
R⁶ is R⁷ or R⁹;
R⁷ is phenyl;
R⁸ is heteroaryl;
R⁹ is heterocycloalkyl;
wherein the moieties represented by R¹, R², R³, R⁴, R⁵ and R⁶ are independently substituted with one or two of independently selected R¹⁰, OR¹⁰, SR¹⁰, SO₂R¹⁰, NH₂, NHC(O)R¹⁰, NHC(O)NHR¹⁰;
R¹⁰ is R¹¹, R¹², R¹³ or R¹⁴;
R¹¹ is phenyl;
R¹² is heteroaryl;
R¹³ is cycloalkyl;
R¹⁴ is alkyl which is unsubstituted or substituted with R¹⁶ or NHC(O)NHR¹⁶;
R¹⁶ is phenyl;
B¹ is H or R¹⁹;
R¹⁹ is R²¹, R²² or R²³;
R²¹ is heteroaryl;
R²² is heterocycloalkyl;
R²³ is alkynyl, which is unsubstituted or substituted with R²⁴, OR²⁴ or N(R²⁴)₂;
R²⁴ is alkyl;
C¹ is S or N(C²);
C² is R³⁰;
R³⁰ is cycloalkyl;
D¹ is N, CH or C(D²);
D² is R³⁷;
R³⁷ is heteroaryl;
wherein each foregoing cyclic moiety is independently unsubstituted or substituted or further unsubstituted or further substituted with one or two or three or four of independently selected R⁴⁴, OR⁴⁴, CN, CF₃, F, Cl, Br or I;
R⁴⁴ is R⁴⁷ or R⁴⁸;
R⁴⁷ is heterocycloalkyl;
R⁴⁸ is alkyl substituted with OP(O)(OH)₂;
wherein R⁴⁷ is unsubstituted or substituted with alkyl.

5. A composition comprising an excipient and a therapeutically effective amount of a compound of claim 1.

6. A method of treating cancer in a mammal comprising administering thereto a therapeutically effective amount of a compound of claim 1.

7. The method of claim 6, wherein the cancer is breast cancer, cervical cancer, colon cancer, endometrial cancer, esophageal cancer, lung cancer, ovarian cancer, pancreatic cancer, prostate cancer, rectal cancer, skin cancer, stomach cancer or thyroid cancer.

8. A method of treating cancer in a mammal comprising administering thereto a therapeutically effective amount of a compound of claim 1 and a therapeutically effective amount of one additional therapeutic agent or more than one additional therapeutic agent, with or without radiation.

9. The method of claim 8, wherein the cancer is breast cancer, cervical cancer, colon cancer, endometrial cancer, esophageal cancer, leukemia, lymphoma, lung cancer, ovarian cancer, pancreatic cancer, prostate cancer, rectal cancer, skin cancer, stomach cancer or thyroid cancer.
